(19) United States
(12) Patent Application Publication
(10) Pub. No.: US 2008/0095774 A1
(43) Pub. Date: Apr. 24, 2008

(54) Agents and Methods for Specifically Blocking CD28-Mediated Signaling

(75) Inventors: Richard M. O'Hara, Quincy, MA (US); Ann Marie Nagelin, Medford, MA (US)

Correspondence Address: FOLEY HOAG, LLP/WYETH;PATENT GROUP, (w/WYS), 155 SEAPORT BLVD., BOSTON, MA, 02210-2600, US

(73) Assignee: Wyeth, Cambridge, MA (US)

(21) Appl. No.: 11/831,517

(22) Filed: Jul. 31, 2007

Related U.S. Application Data
(63) Continuation-in-part of application No. 11/615,686, filed on Dec. 22, 2006, now abandoned.
Application No. 11/615,686 is a continuation-in-part of application No. 10/076,934, filed on Feb. 15, 2002.
(60) Provisional application No. 60/269,756, filed on Feb. 16, 2001.

(51) Int. Cl. A61K 39/395; A61K 35/28; A61P 3/10; A61P 37/06

(52) U.S. Cl. 424/135.1; 424/130.1; 424/133.1; 424/93.7

(57) ABSTRACT

The instant invention provides compositions and methods for downmodulation of immune responses, e.g., autoimmune responses. For example, methods of downmodulating an immune response using agents that specifically block CD28-mediated signaling are provided. The subject methods are useful for both prophylactic and therapeutic downmodulation of immune responses.

### RELATED APPLICATIONS

This application is a continuation in part of U.S. Ser. No. 11/615,686, filed Dec. 22, 2006 which is a continuation-in-part of U.S. Ser. No. 10/076,934, filed Feb. 15, 2002, which claims the benefit of priority to U.S. Ser. No. 60/269,756, filed Feb. 16, 2001. The entire contents of each application are incorporated herein by reference.

### BACKGROUND OF THE INVENTION

In order for T cells to respond to foreign proteins, two signals must be provided by antigen-presenting cells (APCs) to resting T lymphocytes (Jenkins, M. and Schwartz, R. (1987) J. Exp. Med. 165, 302-319; Mueller, D. L., et al. (1990) J. Immunol. 144, 3701-3709). The first signal, which confers specificity to the immune response, is transduced via the T cell receptor (TCR) following recognition of foreign antigenic peptide presented in the context of the major histocompatibility complex (MHC). Polyclonal activators (e.g., anti-CD3 antibodies) can also be used to transmit primary activation signals. The second signal, termed costimulation, induces T cells to proliferate and become functional (Lenschow et al. 1996. Annu. Rev. Immunol. 14:233). Costimulation is neither antigen-specific, nor MHC restricted and is thought to be provided by one or more distinct cell surface molecules expressed by APCs (Jenkins, M. K., et al. 1988 J. Immunol. 140, 3324-3330; Linsley, P. S., et al. 1991 J. Exp. Med. 173, 721-730; Gimmi, C. D., et al., 1991 Proc. Natl. Acad. Sci. USA. 88, 6575-6579; Young, J. W., et al. 1992 J. Clin. Invest. 90, 229-237; Koulova, L., et al. 1991 J. Exp. Med. 173, 759-762; Reiser, H., et al. 1992 Proc. Natl. Acad. Sci. USA. 89, 271-275; van-Seventer, G. A., et al. (1990) J. Immunol. 144, 4579-4586; LaSalle, J. M., et al., 1991 J. Immunol. 147, 774-80; Dustin, M. I., et al., 1989 J. Exp. Med. 169, 503; Armitage, R. J., et al. 1992 Nature 357, 80-82; Liu, Y., et al. 1992 J. Exp. Med. 175, 437-445).

The CD80 (B7-1) and CD86 (B7-2) proteins, expressed on APCs, are critical costimulatory molecules (Freeman et al. 1991. J. Exp. Med. 174:625; Freeman et al. 1989 J. Immunol. 143:2714; Azuma et al. 1993 Nature 366:76; Freeman et al. 1993. Science 262:909). B7-2 appears to play a predominant role during primary immune responses, while B7-1, which is upregulated later in the course of an immune response, may be important in prolonging primary T cell responses or costimulating secondary T cell responses (Bluestone. 1995. Immunity. 2:555).

One ligand to which B7-1 and B7-2 bind, CD28, is constitutively expressed on resting T cells and increases in expression after activation. After signaling through the T cell receptor, ligation of CD28 and transduction of a costimulatory signal induces T cells to proliferate and secrete IL-2 (Linsley, P. S., et al. 1991 J. Exp. Med. 173, 721-730; Gimmi, C. D., et al. 1991 Proc. Natl. Acad. Sci. USA. 88, 6575-6579; June, C. H., et al. 1990 Immunol. Today. 11, 211-6; Harding, F. A., et al. 1992 Nature. 356, 607-609). A second ligand, termed CTLA4 (CD152) is homologous to CD28 but is not expressed on resting T cells and appears following T cell activation (Brunet, J. F., et al., 1987 Nature 328, 267-270). CTLA4 appears to be critical in negative regulation of T cell responses (Waterhouse et al. 1995. Science 270:985). Blockade of CTLA4 has been found to remove inhibitory signals, while aggregation of CTLA4 has been found to provide inhibitory signals that downregulate T cell responses (Allison and Kirummel. 1995. Science 270:932). The B7 molecules have a higher affinity for CTLA4 than for CD28 (Linsley, P. S., et al., 1991 J. Exp. Med. 174, 561-569) and B7-1 and B7-2 have been found to bind to distinct regions of the CTLA4 molecule and have different kinetics of binding to CTLA4 (Linsley et al. 1994 Immunity 1:793). A new molecule related to CD28 and CTLA4, ICOS, has been identified (Hutloff et al. 1999. Nature. 397:263; WO 98/38216).

The importance of the B7:CD28/CTLA4 costimulatory pathway has been demonstrated in vitro and in several in vivo model systems. Blockade of this costimulatory pathway results in the development of antigen specific tolerance in murine and human systems (Harding, F. A., et al. (1992) Nature. 356, 607-609; Lenschow, D. J., et al. (1992) Science. 257, 789-792; Turka, L. A., et al. (1992) Proc. Natl. Acad. Sci. USA. 89, 11102-11105; Gimmi, C. D., et al. (1993) Proc. Natl. Acad. Sci. USA 90, 6586-6590; Boussiotis, V., et al. (1993) J. Exp. Med. 178, 1753-1763). Conversely, expression of B7 by B7 negative murine tumor cells, induces T-cell mediated specific immunity accompanied by tumor rejection and long lasting protection to tumor challenge (Chen, L., et al. (1992) Cell 71, 1093-1102; Townsend, S. E. and Allison, J. P. (1993) Science 259, 368-370; Baskar, S., et al. (1993) Proc. Natl. Acad. Sci. 90, 5687-5690.).

Despite the structural similarities and shared affinity for the ligands B7-1 (CD80) and B7-2 (CD86) it is now clear that CD28 and CTLA-4 (CD152) mediate essentially opposing effects on T cell activation. While the CD28/B7 interaction is known to serve as a positive co-stimulator in the context of TCR engagement by MHC/antigen complex, CTLA-4/B7 is now recognized as imposing a negative effect on cell cycle progression, IL-2 production, and proliferation of T cells following activation.

The development of novel methods for modulating the activities of CD28 and/or CTLA4 would be of great benefit in modulating the immune response. In addition, owing to the opposing effects of engagement of CD28 and CTLA4, specific compositions and methods for separately manipulating one or the other molecule on T cells would be beneficial. In particular, methods of specifically downmodulating T cell responses by modulating the CD28 pathway, while leaving the downmodulatory CTLA4 pathway intact would be beneficial in suppressing immune responses.

### SUMMARY OF THE INVENTION

CD28 has been shown to be important in transmitting a costimulatory signal to T cells and, thereby, regulating T cell activation. The use of anti-CD28 antibodies in the stimulation of immune responses is known in the art (e.g., U.S. Pat. No. 5,948,893). The instant invention is based, at least in part, on the discovery that agents that specifically block CD28-mediated signaling, for example, antigen-binding portions of antibodies, such as scFv molecules, are useful in downmodulating the immune response, both in vitro and in vivo. The instant examples demonstrate that antigen-binding portions of CD28 antibodies are effective in prolonging graft survival in a subject, as well as in preventing the onset of diabetes in NOD mice, a well accepted animal model for the autoimmune disease human type I (immune mediated) diabetes. Both two to three week old animals and adult animals were found to be protected by treatment with anti-CD28 scFv.

Accordingly, in one aspect, the invention relates to a method of therapeutically downmodulating an autoimmune response in a subject by administering an antigen binding portion of an anti-CD28 antibody that blocks signaling via CD28 to the subject such that an autoimmune response in the subject is downmodulated.

In one embodiment, the antigen binding portion is an scFv molecule or an Fab fragment. In certain embodiments, the antigen binding portion is humanized. In another embodiment, the antigen binding portion is fully human.

In another aspect, the invention pertains to a method of therapeutically downmodulating an autoimmune response in a subject comprising administering a small molecule that specifically blocks signaling via CD28 to the subject such that an autoimmune response in the subject is downmodulated.

In one embodiment, the autoimmune response is mediated by CD4+ T cells. In another embodiment, the autoimmune response is mediated by CD8+ T cells.

In one embodiment, the autoimmune response is type I diabetes.

In another aspect, the invention pertains to a method of therapeutically downmodulating an ongoing autoimmune response in a subject by administering an antigen binding portion of an anti-CD28 antibody that blocks signaling via CD28 to the subject such that an ongoing autoimmune response in the subject is downmodulated.

In one embodiment, the antigen binding portion is a scFv molecule or an Fab fragment.

In one embodiment, the antigen-binding portion is humanized. In another embodiment, the antigen-binding portion is fully human.

In still another aspect, the invention pertains to a method of therapeutically downmodulating an ongoing autoimmune response in a subject by administering a small molecule that specifically blocks signaling via CD28 to the subject such that an ongoing autoimmune response in the subject is downmodulated.

In one embodiment, the autoimmune response is mediated by CD4+ T cells. In another embodiment, the autoimmune response is mediated by CD8+ T cells.

In one embodiment, the autoimmune response is type I diabetes.

In another aspect, the invention pertains to a method of prophylactically downmodulating an autoimmune response in a subject by administering an antigen binding portion of an anti-CD28 antibody that blocks signaling via CD28 to the subject such that an autoimmune response in the subject is downmodulated or delayed in its onset.

In one embodiment, the antigen binding portion is a scFv molecule or an Fab fragment.

In one embodiment, the antigen-binding portion is humanized. In another embodiment, the antigen-binding portion is fully human.

In yet another aspect, the invention pertains to a method of prophylactically downmodulating an autoimmune response in a subject comprising administering a small molecule that specifically blocks signaling via CD28 to the subject such that an autoimmune response in the subject is downmodulated or delayed in its onset.

In one embodiment, the autoimmune response is mediated by CD4+ T cells. In another embodiment, the autoimmune response is mediated by CD8+ T cells.

In one embodiment, the autoimmune response is type I diabetes.

In another aspect, the invention relates to methods of prolonging graft survival in a subject in need thereof comprising administering to the subject a non-activating anti-CD28 antibody that blocks CD28 binding to B7 without CD28 signaling such that graft survival in the subject is prolonged.

In one embodiment, the subject is a transplant recipient. In another embodiment, the graft is an allograft such as a cardiac, liver, lung, kidney or pancreatic allograft.

In one embodiment, the non-activating anti-CD28 antibody is an immunologically active fragment. In certain embodiments, the non-activating anti-CD28 antibody is a Fab, F(v), Fab′, or F(ab′)₂. In some embodiments, the non-activating anti-CD28 antibody is a single chain antibody. In some embodiments, the non-activating anti-CD28 antibody is a single chain F(v).

In one embodiment, the anti-CD28 single chain F(v) is linked to an agent to prolong its serum half-life. The agent used to prolong serum half-life may be polyethyleneglycol or a alpha-1-anti-trypsin.

In certain embodiments, an immunosuppressive drug may be administered with a non-activating anti-CD28 antibody. Immunosuppressive drugs that may be co-administered with a non-activating anti-CD28 antibody include, for example, methotrexate, rapamycin, cyclosporin, FK506, an anti-CD154 antibody, a steroid, a CD40 pathway inhibitor, a transplant salvage pathway inhibitor, a IL-2 receptor antagonist, and analogs thereof.

### BRIEF DESCRIPTION OF THE DRAWINGS

FIG. 1 shows that anti-CD28 and PV1 (anti-CD28) scFv bind to CD28 equally.

FIG. 2 shows that PV1 (anti-CD28) scFv inhibits T cell responses in vitro. 1×10⁵ NOD spleen cells were cultured with 1 μg/ml anti-CD3. PV1 scFv or mCTLA4-Ig were added on day 0. Proliferation was measured on day 3.

FIG. 3 shows that PV1 (anti-CD28) scFv prevents disease onset in NOD female mice. Female NOD mice were given intraperitoneal injections of 50 μg anti-CD28 scFv (A) every other day from 2-5 weeks of age within an additional single injection at 6 and 7 weeks of age; and (B) every other day from 8-10 weeks of age. Weekly testing for glucosuria began at 10 weeks of age. Mice were recorded as diabetic after two consecutive positive readings.

FIG. 4 shows that PV1 scFv delays disease onset in adult (8 week old) NOD female mice. Eight week old female NOD mice were injected with 50 μg of PV1 scFv or control antibody for 14 days.

FIG. 5 is a graph showing a pharmacokinetic evaluation of anti-CD28 scFv in vivo. BALB/c mice were treated with 20 mM KI in drinking water for 3 days prior to study initiation. At dosing, mice were injected with a mixture of 125I labeled an unlabeled anti-CD28 scFv, at a total dose of 1 mg/kg. Three animals were bled by cardiac puncture at 5 minutes, 15 minutes, 1, 3, 6, 24, 48 and 72 hours. Blood samples were assayed for radioactivity.

FIG. 6 is a FACS analysis showing that anti-CD29scFv is readily detectable on peripheral T cells. Mice were injected intraperitoneally with 50 μg anti-CD28 single chain antibody or control Fab. Two hours after injection, peripheral blood, spleen and lymph node samples were harvested and stained for the presence of antibody. Single chain antibody is detectable on peripheral T cells in blood, spleen and lymph node. Samples taken at 18 hours after injection did not show detectable single chain antibody on the cell surface.

FIG. 7 is a FACS analysis showing no increase in Treg cell numbers in anti-CD28 single chain treated mice. 1×10⁶ spleen cells from NOD mice treated with anti-CD28 scFv were stained with anti-CD4-FITC and anti-CD25-PE to detect regulatory T cells. Representative FACS plots of spleen cells from untreated mice (A), mice treated with anti-CD28 scFv as weanlings (every other day from 2-5 weeks of age with additional injections at 6 and 7 weeks) (B) or mCTLA4-Ig from 8-10 weeks of age (C) are shown. Percent of spleen cells staining for CD4 and CD25 from individual animals treated with anti-CD28 scFv as weanlings (D), or with anti-CD28 scFv from 8-10 weeks of age (E) or with mCTLA4-Ig form 8-10 weeks of age (F) are shown compared to appropriate controls.

FIG. 8 is a FACS analysis showing increase glucose tolerance in anti-CD28 scFv treated mice. Recent onset diabetic NOD females (first positive urine glucose test within one week following an negative test) were injected with control Fab or anti-CD28 scFv (50 μg daily intraperitoneal injections) for seven days. Glucose tolerance tests were performed on days 0, 2, 4 and 7. Data from individual animals are plotted as AUC measurements for results of the 90 minute test over the seven day period (A). (B) Comparison of control Fab treated mice on day 0 and day 7. Note that in all cases, control Fab treated mice have poorer GTT results on day 7 of treatment compared to day 0. (C) Comparison of anti-CD28 scFv treated mice on day 0 and day 7. Note that 4 of 10 mice demonstrated improved GTT results on day 7 of treatment compared to day 0.

FIG. 9 shows selective CD28 blockade inhibits allogeneic T cell proliferation in vitro. Physiologically relevant concentrations of various anti-CD28 scFv reagents inhibit allogeneic mixed lymphocyte proliferation in a dose dependent manner in mouse (a,c), cynomolgus monkey (b,d) and human (e). In mice, CD154 blockade with MR1 at 20 μg/ml enhanced the antiproliferative effect of αm28scFv (c); a similar effect was not observed for monkey cells using IDEC-131 at 10 μg/ml (d). Results are representative of 2-4 independent experiments. (e) Blocking CTLA4 with a specific anti-CTLA4 antibody (BNI3) restores cell proliferation of human T cells cultured in the presence of αh28scAT or Fab fragments from anti-CD28.3 antibody (data not shown), demonstrating that the anti-proliferative effect of selective CD28 blockade is actively mediated by CTLA4. Control: 10 μg/ml α1-anti-trypsine (AT); α1h28scAT+mIgG: 10 μg/ml αh28scAT plus 25 μg/ml mouse IgG1; α1h28scAT+anti-CTLA4: 10 μg/ml αh28scAT plus 25 μg/ml anti-CTLA-4 BNI3 Mab; anti-CTLA-4: 25 μg/ml anti-CTLA-4 BNI3 Mab. Data are means±SEM of 5 independent mixed lymphocyte reactions. Mouse splenocytes and monkey or human peripheral blood mononuclear cells were isolated and tested in MLR as described in Methods. (*: p<0.05).

FIG. 10 shows selective CD28 inhibition prolongs allograft survival and prevents chronic rejection in mice. BALB/c recipients received fully MHC-mismatched C57BL/6 heterotopic cardiac allografts or BALB/c isografts as controls. Allograft recipients were treated with αm28scFv (200 μg, d0-13), MR1 (250 μg, d0), CsA (400 μg, d0-3) or combinations as described in Methods. (a) αm28scFv prolongs graft survival, an effect significantly augmented when combined with transient CD154 blockade or calcineurin inhibition with CsA. Color coding corresponds to treatment groups. (b) Representative arteries in surviving grafts over 100 days after transplant, demonstrating the effect of CD28 blockade on chronic rejection (Verhoeff's elastin staining, original magnification ×200). An MR1 treated cardiac allograft shows grade 3 CAV (>50% luminal occlusion) with severe intimal thickening (arrow) and a mild-moderate perivascular and neointimal cellular infiltrate. In contrast, grafts treated with αm28scFv combined with either MR1 or CsA show absence of neointimal proliferation (arrows). (c) Incidence and (d) severity of CAV measured as the proportion of vessels exhibiting a CAV score >1, and mean CAV score, graded for neointimal thickening as described in Methods. αm28scFv with either CsA or MR1 was associated with markedly less neointimal thickening characteristic of CAV relative to MR1 alone.

FIG. 11 shows representative histological analysis of mouse cardiac allografts two weeks after transplant (H&E staining). Intense cellular infiltrate edema and hemorrhage are prominent in rejected untreated controls, and are only partially prevented with CsA, αm28scFv alone, or MR1 alone. In contrast, pristine heart structure and scant mononuclear cell infiltration are associated with αm28scFv combined with MR1 or CsA.

FIG. 12 shows the mechanism of immune modulation by selective CD28 blockade. (a) Th2 (IgG1) and Th1 (IgG2a) alloantibody production measured early (d10-15) and after d100 following transplantation as described in Methods. Early elaboration of both Th1 and Th2 alloantibody was decreased in αm28scFv-based combined treatment regimens. At day 100, Th2 alloantibody production was prevalent in association with both chronic rejection (MR1 alone) and, combined regimens whereas Th1 alloantibody was rarely detected, suggesting that prolonged graft acceptance following costimulation blockade is associated with modulation of this limb of the anti-donor antibody response. (b) Frequency of alloantigen-specific cytokine-producing splenocytes in recipients treated with various therapies, measured by ELISPOT early (d10-15) or after day 100 as described in Methods. Differences in Th1 precursor number between untreated animals or each monotherapy group versus the early MR1 and late CsA combined treatment groups achieve statistical significance. αm28scFv with CsA or MR1 animals tended to exhibit lower early anti-donor expansion than animals from groups which typically succumb to acute rejection. However neither early nor late cytokine precursor profiles in the spleen clearly distinguish between chronic rejection and tolerance. In animals with accepted grafts detectable Th1 anti-donor responses are prevalent in spleen after day 100; IL-10 producing cells were also detected at 100 days with MR1 or αm28scFv+MR1 treatment. (c) Increased proportion of Foxp3⁺CD4⁺ T cells at day 10-12 in graft infiltrating cells isolated from recipients treated with αm28scFv and MR1 or CsA relative to native heart (naive), acutely rejecting grafts without treatment (No Rx), or with αm28scFv monotherapy (am28scFv). Graft infiltrating cells (GILs) were isolated as described in Methods and stained for surface CD3, CD4, CD25 and intra-cellular Foxp3. Results are expressed as the proportion of CD4⁺ Foxp3⁺ cells among graft infiltrating CD3+ T-cells. Top: Representative FACS scatter plot; Bottom: Each dot represents an individual animal, the bar displays the group mean and box-and-whisker representation displays the mean and 25^{th} and 75^{th} quartiles (box).

FIG. 13 shows skin graft survivial in long-term heart graft-accepting recipients. Representative examples of skin graft transplants tested in Balb/c recipients 100 days after transplantation. C3H skin was used as third party control for C57BL/6 donor-type skin. No immunosuppression was administered at the time of skin transplantation. Inserts indicated the induction regimen used for cardiac allografts, and the time after skin transplantation. Results are summarized in Table 2.

FIG. 14 shows Th1/Th2 cytokine rations calculated from the ELISPOT results for each animal and depicted as mean±SD.

FIG. 15 shows selective CD28 inhibition prolongs allograft survival and prevents chronic rejection in non-human primates. Wild-caught cynomolgus monkeys recipients of MHC-mismatched heterotopic cardiac allografts were either untreated (grey), or treated with αh28scAT monotherapy (light blue), therapeutic CsA (pink), or αh28scAT with therapeutic CsA (dark blue); αh28scAT treatment frequency and dose are indicated. Graft survival was monitored by telemetric ECG and pressure waveforms. (a) CD28 blockade alone (n=3) prolonged graft survival relative to no treatment (n=5, p=0.01). (b) A representative vessel from a cardiac allograft treated with CsA (M9421, day 72) shows grade 2 CAV with distinct neointimal thickening and 10-50% (estimated at 25% in this instance) luminal narrowing. In contrast, a representative graft artery from a recipient treated with αh28scAT and CsA shows absence of neointimal proliferation (M9429, day 80). (Verhoeff s elastin staining, original magnification ×200.) CAV incidence (c) and (d) severity, graded as described in Methods, were significantly lower in association with CD28 blockade with CsA compared to CsA alone.

### DETAILED DESCRIPTION OF THE INVENTION

The instant invention pertains, at least in part, to methods of downmodulating the immune response using molecules that specifically block CD28-mediated signaling, e.g., scFv of anti-CD28 antibodies.

In one aspect, the invention provides selective inhibition of CD28 function during initial antigen exposure as a method of promoting immune tolerance. Non-activating single chain Fv-based reagents, transiently blocked CD28 interactions during engraftment and promoted prolonged graft acceptance in both mouse and monkey heart transplant models. As described herein, anti-CD28 with a marginally effective dose of CD154-blocking antibody or subtherapeutic Cyclosporin A (CsA) induced robust donor-specific transplant tolerance, an effect abrogated by additional CTLA-4 blockade in mice. Graft acceptance with anti-CD28 was associated with early (day 10-15) graft infiltration by Foxp3+ regulatory T-cells and increased late (>day 100) expression of genes associated with regulatory T-cells (Foxp3 and CTLA-4) and dendritic cells (IDO). Also as described herein, CD28 blockade at induction, added to calcineurin-based immunosuppression, significantly attenuated chronic rejection in monkeys.

In another aspect of the invention, non-activating single chain Fv-based reagents prevented the onset of diabetes in NOD mice, a well accepted animal model for the autoimmune disease human type I (immune mediated) diabetes. Both two to three week old animals and adult animals were found to be protected by treatment with anti-CD28 scFv.

Various aspects of the invention are described in further detail in the following subsections:

I. Definitions

The term “allograft” as used herein refers to the transplant of an organ or tissue from one individual to another individual of the same species with a different genotype. An allograft may be a cardiac, liver, lung, kidney, pancreatic or other organ or tissue allograft. An allograft may also be referred to as an allogenic graft or a homograft.

The term “subject” as used herein refers to vertebrate hosts, particularly to mammals, and includes, but is not limited to, primates, including humans, and domestic animals.

As used herein, the term “immune response” includes T cell mediated and/or B cell mediated immune responses that are influenced by modulation of T cell costimulation. Exemplary immune responses include T cell responses, e.g., proliferation, cytokine production, and cellular cytotoxicity. In addition, the term immune response includes immune responses that are indirectly effected by T cell activation, e.g., antibody production (humoral responses) and activation of cytokine responsive cells, e.g., macrophages.

As used herein, the term “primary immune response” includes immune responses to antigens which have not been seen before by a subject, e.g., to which the subject is naive.

As used herein, the term “secondary immune response” includes immune responses to antigens which have been seen before by a subject, e.g., to which the subject has been primed. The tem “ongoing immune response” includes an immune response to a certain antigen which is ongoing, e.g, is presently active and detectable.

As used herein, the term “prophylactically” includes the administration of an effective molecule of the invention before the onset of an undesirable immune response.

As used herein, the term “therapeutically” includes the administration of an effective molecule of the invention to treat an existing or ongoing unwanted immune response (e.g., an autoimmune response) which would benefit by treatment with the agent.

As used here, the term “self” with reference to a peptide includes peptides which are not foreign to a subject and to which an autoimmune response can occur. The immune system can normally discriminate between self and non-self (“foreign”). Optimally, the mammalian immune system is non-reactive (e.g., tolerant) to self-antigens. The mechanisms that provide tolerance normally eliminate or render inactive clones of B and T cells that would otherwise carry out anti-self reactions. Autoimmune diseases or disorders (e.g., multiple sclerosis, rheumatoid arthritis, lupus erythematosus, and Type I diabetes mellitus) represent an aberrant immune attack in which antibodies or T cells of a host are directed against self-antigen not normally the target of the immune response. Autoimmunity results from the dysfunction of normal mechanisms of self-tolerance that prevent the production of functional self-reactive clones of B and T cells.

As used herein, the term “costimulate” with reference to activated T cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. T cells that have received a cell-receptor mediated signal, e.g., via a T cell receptor (TCR) (e.g., by an antigen or by a polyclonal activator) are referred to herein as “activated T cells.”

For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes, and expression of activation markers, e.g., CTLA4.

Transmission of a costimulatory signal to a T cell (e.g., via cross-linked CD28 molecules) involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death in the T cell.

A “CD28-mediated signal” includes one or more cellular events directly or indirectly induced in an immune cell which expresses CD28 on its surface by the binding of a ligand that activates (e.g., crosslinks) the cell surface CD28. Activation of CD28 receptor(s) triggers a signaling event(s) which results in a measurable cellular change. CD28-mediated signaling can be detected, for instance, by measuring commonly measured parameters of T cell costimulation in an in vitro assay. Under the appropriate circumstances CD28-mediated signaling results in the upmodulation of an immune response by the immune cell. Blockade of CD28-mediated signaling results in the downmodulation of an immune response by the immune cell. An agent which binds to CD28 to effectively block a CD28-mediated signal (e.g., by blocking ligand binding) without itself activating the CD28 receptor (e.g., via aggregation of the receptor) will effectively block CD28-mediated signaling. Preferably, an agent specifically blocks CD28-mediated signaling, i.e., blocks a signal transmitted by CD28, while not blocking a signal transmitted by another cell surface molecule, e.g., CTLA4.

As used herein, the term “inhibitory signal” refers to a signal transmitted via an inhibitory receptor (e.g., CTLA4) on an immune cell. Such a signal antagonizes a signal transmitted via an activating receptor (e.g., via a TCR) and can result in, e.g., inhibition of second messenger generation; inhibition of proliferation; inhibition of effector function in the immune cell, (e.g., reduced cellular cytotoxicity) the failure of the immune cell to produce mediators, (such as cytokines (e.g., IL-2) and/or mediators of allergic responses); or the development of anergy.

As used herein, the term “unresponsiveness” includes refractivity of immune cells to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. 1992. Science. 257:1134).

As used herein, the term “activity” with respect to a polypeptide includes activities which are inherent in the structure of a polypeptide. With respect to CD28, the term “activity” includes the ability of a CD28 polypeptide to bind to a costimulatory molecule (e.g., CD80 or CD86) and/or to modulate a costimulatory signal in an activated immune cell, e.g., by engaging a natural ligand on an antigen presenting cell. CD28 transmits a costimulatory signal to a T cell. Modulation of an costimulatory signal in a T cell results in modulation of proliferation of and/or cytokine secretion by the T cell. CD28 can also modulate a costimulatory signal by competing with an inhibitory receptor for binding of costimulatory molecules, e.g., CTLA4. Thus, the term “CD28 activity” includes the ability of a CD28 polypeptide to bind its natural ligand(s), the ability to modulate immune cell costimulatory or inhibitory signals, and the ability to modulate the immune response.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The phrase “complementary determining region” (CDR) includes the region of an antibody molecule which comprises the antigen binding site.

The antibody may be an IgG such as IgG1, IgG2, IgG3 or IgG4; or IgM, IgA, IgE or IgD isotype. The constant domain of the antibody heavy chain may be selected depending upon the effector function desired. The light chain constant domain may be a kappa or lambda constant domain.

The term “antigen-binding portion”, as used herein, refers to one or more fragments of an antibody that retain the ability to specifically bind to an antigen (e.g. human CD28). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see e.g., Bird et al. (1988) Science 242:423-426; and Huston et al. (1988) Proc. Natl. Acad. Sci. USA 85:5879-5883). Such single chain antibodies (scFvs) are preferred molecules intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g. Holliger, P., et al. (1993) Proc. Natl. Acad. Sci. USA 90:6444-6448; Poljak, R. J., et al. (1994) Structure 2:1121-1123). Preferably, the antigen-binding fragments do not cross-link the antigen to which they bind.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Antibodies may be polyclonal or monoclonal; xenogeneic, allogeneic, or syngeneic; or modified forms thereof, e.g. humanized, chimeric, etc. Preferably, antibodies of the invention bind specifically or substantially specifically to CD28 molecules present on a T cell of a subject. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

An “isolated antibody”, as used herein, is intended to refer to an antibody that is substantially free of other antibodies having different antigenic specificities (e.g. an isolated antibody that specifically binds CD28 is substantially free of antibodies that specifically bind antigens other than CD28). Moreover, an isolated antibody may be substantially free of other cellular material and/or chemicals.

“Anti-CD28 antibodies” are antibodies that specifically bind to a site on the extracellular domain of CD28 protein, and modulate a costimulatory signal to a T cell. The term “anti-CD28 antibodies” includes antibodies that block the binding of CD28 to costimulatory molecules, e.g. CD80 and/or CD86.

The phrase “specifically” with reference to binding, recognition, or reactivity of antibodies includes antibodies which bind to naturally occurring molecules which are expressed transiently only on activated T cells. In particular, with respect to CD28, the term “specifically” with reference to binding, recognition, or reactivity of antibodies includes anti-CD28 antibodies that bind to naturally occurring forms of CD28, but are substantially unreactive with molecules related to CD28, such as CTLA4 and other members of the immunoglobulin superfamily. The phrase “substantially unreactive” includes antibodies which display no greater binding to molecules related to CD28, e.g., CTLA4 (but excluding CD28 molecules) as compared to unrelated molecules, e.g., CD27. Preferably, such antibodies bind to molecules related to CD28 (but excluding CD28 molecules) with only background binding. Antibodies specific for CD28 from one source, e.g., human CD28 may or may not be reactive with CD28 molecules from different species. Antibodies specific for naturally occurring CD28 may or may not bind to mutant forms of such molecules. In one embodiment, mutations in the amino acid sequence of a naturally occurring CD28 molecule result in modulation of the binding (e.g., either increased or decreased binding) of the antibody to the CD28 molecule. Antibodies to CD28 can be readily screened for their ability to meet this criteria. Assays to determine affinity and specificity of binding are known in the art, including competitive and non-competitive assays. Assays of interest include ELISA, RIA, flow cytometry, etc. Binding assays may use purified or semi-purified CD28 protein, or alternatively may use cells that express CD28, e.g. cells transfected with an expression construct for CD28; T cells that have been stimulated through cross-linking of CD3 or the addition of irradiated allogeneic cells, etc. As an example of a binding assay, purified CD28 protein is bound to an insoluble support, e.g. microtiter plate, magnetic beads, etc. The candidate antibody and soluble, labeled CD80 or CD86 are added to the cells, and the unbound components are then washed off. The ability of the antibody to compete with CD80 and CD86 for CD28 binding is determined by quantitation of bound, labeled CD80 or CD86. Confirmation that the blocking agent does not cross-react with CTLA4 may be performed with a similar assay, substituting CTLA4 for CD28. An isolated antibody that specifically binds human CD28 may, however, have cross-reactivity to other antigens, such as CD28 molecules from other species.

Antigen binding portions of anti-CD28 antibodies can be administered to patients or cells of a patient can be caused to express such molecules, e.g., in soluble form. As used herein, the term “causing to express” with reference to an antibody or antibody biding portion includes art recognized methods by which a cell can be made to express a particular molecule. For example, methods such as transfection can be used to cause a cell to express an antigen binding portion of an anti-CD28 molecule (e.g., an antigen binding portion of an anti-CD28 antibody or an MHC molecule).

For example, DNA can be introduced into cells of a subject via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

As used herein, a “naturally-occurring” nucleic acid molecule refers to an RNA or DNA molecule having a nucleotide sequence that occurs in nature (e.g., encodes a natural protein).

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “vector” refers to a nucleic acid molecule capable of transporting another nucleic acid molecule to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions.

As used herein, the term “host cell” is intended to refer to a cell into which a nucleic acid molecule of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, an “isolated protein” refers to a protein that is substantially free of other proteins, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the protein is derived, or substantially free from chemical precursors or other chemicals when chemically synthesized. The language “substantially free of cellular material” includes preparations of protein in which the protein is separated from cellular components of the cells from which it is isolated or recombinantly produced. In one embodiment, the language “substantially free of cellular material” includes preparations of protein having less than about 30% (by dry weight) of contaminating protein (e.g., non-CD28 or non-anti-CD28 antibody), more preferably less than about 20% of contaminating protein, still more preferably less than about 10% of contaminating protein, and most preferably less than about 5% contaminating protein. When the CD28 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or contaminating chemicals, more preferably less than about 20% chemical precursors or contaminating chemicals, still more preferably less than about 10% chemical precursors or contaminating chemicals, and most preferably less than about 5% chemical precursors or contaminating chemicals.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a CD28 polypeptide or CD28 antibody of the invention (or any portion thereof) can be used to derive the CD28 polypeptide amino acid sequence or CD28 antibody amino acid sequence, using the genetic code to translate the CD28 polypeptide or CD28 antibody molecule into an amino acid sequence. Likewise, for any CD28 polypeptide or CD28 antibody-amino acid sequence, corresponding nucleotide sequences that can encode CD28 polypeptide or CD28 antibody protein can be deduced from the genetic code (which, because of its redundancy, will produce multiple nucleic acid sequences for any given amino acid sequence).

Thus, description and/or disclosure herein of a nucleotide sequence encoding a CD28 polypeptide or a nucleotide sequence encoding a CD28 antibody should be considered to also include description and/or disclosure of the amino acid sequence encoded by the nucleotide sequence. Similarly, description and/or disclosure of a CD28 polypeptide or CD28 antibody amino acid sequence herein should be considered to also include description and/or disclosure of all possible nucleotide sequences that can encode the amino acid sequence.

II. Agents that Specifically Block CD28-Mediated Signaling

A. Anti-CD28 Antibodies

Antibodies typically comprise two heavy chains linked together by disulfide bonds and two light chains. Each light chain is linked to a respective heavy chain by disulfide bonds. Each heavy chain has at one end a variable domain followed by a number of constant domains. Each light chain has a variable domain at one end and a constant domain at its other end. The light chain variable domain is aligned with the variable domain of the heavy chain. The light chain constant domain is aligned with the first constant domain of the heavy chain. The constant domains in the light and heavy chains are not involved directly in binding the antibody to antigen. The variable domains of each pair of light and heavy chains form the antigen binding site.

The domains on the light and heavy chains have the same general structure and each domain comprises a framework of four regions, whose sequences are relatively conserved, connected by three complementarity determining regions (CDRs). The four framework regions largely adopt a beta-sheet conformation and the CDRs form loops connecting, and in some cases forming part of, the beta-sheet structure. The CDRs are held in close proximity by the framework regions and, with the CDRs from the other domain, contribute to the formation of the antigen binding site. CDRs and framework regions of antibodies may be determined by reference to Kabat et al (“Sequences of proteins of immunological interest” US Dept. of Health and Human Services, US Government Printing Office, 1987).

Polyclonal anti-CD28 antibodies can be prepared as described above by immunizing a suitable subject with a CD28 immunogen. The anti-CD28 antibody titer in the immunized subject can be monitored over time by standard techniques, such as with an enzyme linked immunosorbent assay (ELISA) using immobilized a CD28 polypeptide. If desired, the antibody molecules directed against a CD28 polypeptide can be isolated from the mammal (e.g. from the blood) and further purified by well known techniques, such as protein A chromatography to obtain the IgG fraction. At an appropriate time after immunization, e.g., when the anti-CD28 antibody titers are highest, antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a CD28 immunogen as described above, and the culture supernatants of the resulting hybridoma cells are screened to identify a hybridoma producing a monoclonal antibody that binds specifically to a CD28 polypeptide.

Any of the many well known protocols used for fusing lymphocytes and immortalized cell lines can be applied for the purpose of generating an anti-CD28 monoclonal antibody (see, e.g. G. Galfre et al. (1977) Nature 266:550-52; Gefter et al. Somatic Cell Genet., cited supra; Lerner, Yale J. Biol. Med., cited supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a CD28 molecule, e.g. using a standard ELISA assay.

As an alternative to preparing monoclonal antibody-secreting hybridomas, a monoclonal anti-CD28 antibody can be identified and isolated by screening a recombinant combinatorial immunoglobulin library (e.g., an antibody phage display library) with CD28 (or a portion of a CD28 molecule, e.g., the extracellular domain of CD28) to thereby isolate immunoglobulin library members that bind a CD28 polypeptide. Kits for generating and screening phage display libraries are commercially available (e.g. the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Anti-CD28 antibodies may bind to any portion of the CD28 molecule such that binding of CD28 to CD80 and/or CD86 is modulated upon the binding of the antibody to CD28. Preferably, anti-CD28 antibodies bind to the extracellular domain of the CD28 molecule.

An exemplary anti-CD28 antibody for use in the instant invention is the anti-human CD28 antibody made in a non-human animal, e.g., a rodent. Anti-CD28 antibodies are known in the art, see e.g., U.S. Pat. No. 5,948,893.

### Preparation of Anti-CD28 Antibodies

CD28 Immunogens

One aspect of the invention pertains to anti-CD28 antibodies. Antibodies to CD28 can be made by immunizing a subject (e.g., a mammal) with a CD28 polypeptide or a nucleic acid molecule encoding a CD28 polypeptide or a portion thereof. In one embodiment, native CD28 proteins, or immunogenic portions thereof, can be isolated from cells or tissue sources by an appropriate purification scheme using standard protein purification techniques. In another embodiment, CD28 proteins, or immunogenic portions thereof, can be produced by recombinant DNA techniques. Alternative to recombinant expression, a CD28 protein or immunogenic portion thereof, can be synthesized chemically using standard peptide synthesis techniques. Alternatively, nucleic acid molecules encoding a CD28 molecule or portion thereof can be used as immunogens. Whole cells expressing CD28 can be used as immunogens to produce anti-CD28 antibodies.

The origin of the immunogen may be mouse, human, rat, monkey etc. The host animal will generally be a different species than the immunogen, e.g. mouse CD28 used to immunize hamsters, human CD28 to immunize mice, etc. The human and mouse CD28 contain highly conserved stretches in the extracellular domain (Harper et al. (1991) J. Immunol. 147:1037-1044). Peptides derived from such highly conserved regions may be used as immunogens to generate cross-specific antibodies. The nucleotide and amino acid sequences of CD28 from a variety of sources are known in the art and can be found, for example in Proc. Natl. Acad. Sci. U.S.A. 84 (23), 8573-8577 (1987) and J. Immunol. 145:344 (1990); GenBank accession number NM 006139.

In one embodiment, the immunogen may comprise the complete protein, or fragments and derivatives thereof. Preferred immunogens comprise all or a part of the extracellular domain of human CD28 where these residues contain the post-translation modifications, such as glycosylation, found on the native CD28. Immunogens comprising the extracellular domain are produced in a variety of ways known in the art, e.g. expression of cloned genes using conventional recombinant methods, isolation from T cells, sorted cell populations expressing high levels of CD28, etc. In another embodiment, the immunogen may comprise DNA encoding a CD28 molecule or a portion thereof. For example, as set forth in the appended examples, 2 μg cDNA encoding the extracellular domain of recombinant human CD28 could be used as an immunogen.

In a preferred embodiment, the immunogen is a human CD28 molecule. Preferably, CD28 proteins comprise the amino acid sequence encoded by SEQ ID NO: 1 or fragment thereof. In another preferred embodiment, the protein comprises the amino acid sequence of SEQ ID NO: 2 or fragment thereof. For example, the CD28 molecule can differ in amino acid sequence from that shown in SEQ ID NO:2, e.g., can be from a different source or can be modified to increase its immunogenicity. In one embodiment, the protein has at least about 80%, and even more preferably, at least about 90% or 95% amino acid identity with the amino acid sequence shown in SEQ ID NO: 2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which need to be introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. In a preferred embodiment, the percent identity between two amino acid sequences is determined using the readily available GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6.

The nucleic acid and protein sequences of the CD28 can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to CD28 nucleic acid molecules of the invention. BLAST protein searches can be performed with the XBLAST program, score=50, wordlength=3 to obtain amino acid sequences homologous to CD28 protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See, e.g., the NCBI web page.

CD28 chimeric or fusion proteins or nucleic acid molecules encoding them can also be used as immunogens. As used herein, a CD28 “chimeric protein” or “fusion protein” comprises a CD28 polypeptide operatively linked to a non-CD28 polypeptide. A “CD28 polypeptide” refers to a polypeptide having an amino acid sequence corresponding to CD28 polypeptide, whereas a “non-CD28 polypeptide” refers to a polypeptide having an amino acid sequence corresponding to a protein which is not substantially homologous to the CD28 protein, e.g., a protein which is different from the CD28 protein and which is derived from the same or a different organism. Within a CD28 fusion protein the CD28 polypeptide can correspond to all or a portion of a CD28 protein. In a preferred embodiment, a CD28 fusion protein comprises at least one biologically active portion of a CD28 protein, e.g., an extracellular domain of a CD28 protein. Within the fusion protein, the term “operatively linked” is intended to indicate that the CD28 polypeptide and the non-CD28 polypeptide are fused in-frame to each other. The non-CD28 polypeptide can be fused to the N-terminus or C-terminus of the CD28 polypeptide.

Preferably, a CD28 fusion protein or nucleic acid molecule encoding a CD28 fusion protein is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A CD28 encoding nucleic acid molecule can be cloned into such an expression vector such that the fusion moiety is linked in-frame to the CD28 protein. Such fusion moieties can be linked to the C or to the N terminus of the CD28 protein or a portion thereof.

Variants of the CD28 proteins can also be generated by mutagenesis, e.g., discrete point mutation or truncation of a CD28 protein and used as a immunogen. In one embodiment, variants of a CD28 protein can be identified by screening combinatorial libraries of mutants, e.g., truncation mutants, of a CD28 protein for CD28 protein agonist or antagonist activity. In one embodiment, a variegated library of CD28 variants is generated by combinatorial mutagenesis at the nucleic acid level and is encoded by a variegated gene library. A variegated library of CD28 variants can be produced by, for example, enzymatically ligating a mixture of synthetic oligonucleotides into gene sequences such that a degenerate set of potential CD28 sequences is expressible as individual polypeptides, or alternatively, as a set of larger fusion proteins (e.g., for phage display) containing the set of CD28 sequences therein. There are a variety of methods which can be used to produce libraries of potential CD28 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CD28 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a CD28 protein coding sequence can be used to generate a variegated population of CD28 fragments for screening and subsequent selection of variants of a CD28 protein. In one embodiment, a library of coding sequence fragments can be generated by treating a double stranded PCR fragment of a CD28 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S 1 nuclease, and ligating the resulting fragment library into an expression vector. By this method, an expression library can be derived which encodes N-terminal, C-terminal and internal fragments of various sizes of the CD28 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CD28 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated CD28 library. For example, a library of expression vectors can be transfected into a cell line which ordinarily synthesizes CD28. The transfected cells are then cultured such that CD28 and a particular mutant CD28 are made and the effect of expression of the mutant on CD28 activity in cell supernatants can be detected, e.g., by any of a number of costimulatory assays. Plasmid DNA can then be recovered from the cells which score for inhibition, or alternatively, potentiation of CD28 activity, and the individual clones further characterized.

An isolated CD28 protein, or a portion or fragment thereof, or nucleic acid molecules encoding a CD28 polypeptide of portion thereof, can be used as an immunogen to generate antibodies that bind CD28 using standard techniques for polyclonal and monoclonal antibody preparation. In one embodiment, a full-length CD28 protein or nucleic acid molecule encoding a full-length CD28 protein can be used. Alternatively, an antigenic peptide fragment (i.e., a fragment capable of promoting an antigenic response) of a CD28 polypeptide or nucleic acid molecule encoding a fragment of a CD28 polypeptide can be used can be used as the immunogen. An antigenic peptide fragment of a CD28 polypeptide typically comprises at least 8 amino acid residues (e.g., at least 8 amino acid residues of the amino acid sequence shown in SEQ ID NO: 2) and encompasses an epitope of a CD28 polypeptide such that an antibody raised against the peptide forms an immune complex with a CD28 molecule. Preferred epitopes encompassed by the antigenic peptide are regions of CD28 that are located on the surface of the protein, e.g., hydrophilic regions. In another embodiment, an antibody binds specifically to a CD28 polypeptide. In a preferred embodiment, the CD28 polypeptide is a human CD28 polypeptide.

Preferably, the antigenic peptide comprises at least about 10 amino acid residues, more preferably at least about 15 amino acid residues, even more preferably at least about 20 amino acid residues, and most preferably at least about 30 amino acid residues. Preferred epitopes encompassed by the antigenic peptide are regions of a CD28 polypeptide that are located on the surface of the protein, e.g., hydrophilic regions, and that are unique to a CD28 polypeptide. In one embodiment such epitopes can be specific for a CD28 proteins from one species, such as mouse or human (i.e., an antigenic peptide that spans a region of a CD28 polypeptide that is not conserved across species is used as immunogen; such non conserved residues can be determined using an amino acid sequence, e.g., using one of the programs described supra). A standard hydrophobicity analysis of the CD28 protein can be performed to identify hydrophilic regions.

A CD28 immunogen can be used to prepare antibodies by immunizing a suitable subject, (e.g., rabbit, goat, mouse or other mammal) with the immunogen. An appropriate immunogenic preparation can contain, for example, a nucleic acid molecule encoding a CD28 immunogen, a recombinantly expressed CD28 protein or a chemically synthesized CD28 immunogen. The preparation can further include an adjuvant, such as Freund's complete or incomplete adjuvant, alum, a cytokine or cytokines, or similar immunostimulatory agent. Immunization of a suitable subject with an immunogenic CD28 preparation induces a polyclonal anti-CD28 antibody response.

Alteration of Antibodies

A variety of different alterations or changes can be introduced into the subject antibodies to optimize their use in downmodulating the immune response. For example, mutations can be introduced into constant and/or variable regions to preserve or enhance e.g., affinity, specificity, and/or half life optionally, alteration may be introduced to decrease immunogenicity. For example, conservative amino acid substitutions can be made. Exemplary changes include: substitution of isoleucine, valine, and leucine for any other of these hydrophoic amino acids. Aspartic acid can be substituted for glutamic acid and vice versa. Glutamine can be substituted for asparagine and vice versa. Serine can be substituted for threonine and vice versa. Other substitutions can also be considered to be conservative, depending on the environment of the particular amino acid and its role in the three-dimensional structure of the protein. For example, glycine and alanine can be interchangeable, as can alanine and valine. Methionine, which is relatively hydrophobic, can often be interchanged with leucine and isoleucine, and sometimes with valine. Lysine and arginine can be interchangeable in locations in which the significant feature of the amino acid residue is its charge and the differing pK's of the two amino acid residues are not significant. Changes that do not affect the three-dimensional structure or the reactivity of the protein can be determined by computer modeling.

For in vivo use, particularly for injection into humans, it is often desirable to decrease the antigenicity of an antibody. An immune response of a recipient against the blocking agent will potentially decrease the period of time that the therapy is effective. To minimize such an immune response, humanized or chimeric antibodies can be constructed. Various methods of humanizing antibodies can be used. For example, the humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) P.N.A.S. 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Additionally, recombinant anti-CD28 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060. In addition, humanized antibodies can be made according to standard protocols such as those disclosed in U.S. Pat. Nos. 5,777,085; 5,530,101; 5,693,762; 5,693,761; 5,882,644; 5,834,597; 5,932,448; or 5,565,332.

For example, an antibody may be humanized by grafting the desired CDRs onto a human framework, e.g., according to EP-A-0239400. A DNA sequence encoding the desired reshaped antibody can be made beginning with the human DNA whose CDRs it is wished to reshape. The rodent variable domain amino acid sequence containing the desired CDRs is compared to that of the chosen human antibody variable domain sequence. The residues in the human variable domain are marked that need to be changed to the corresponding residue in the rodent to make the human variable region incorporate the rodent CDRs. There may also be residues that need substituting, e.g., adding to or deleting from the human sequence. Oligonucleotides can be synthesized that can be used to mutagenize the human variable domain framework to contain the desired residues. Those oligonucleotides can be of any convenient size.

Alternatively, humanization may be achieved using the recombinant polymerase chain reaction (PCR) methodology taught, e.g., in WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

Construction of scFv Antigen Binding Portions of Anti-CD28 Antibodies

Single-chain Fv (ScFv) molecules are antigen binding portions in which the VH and VL partner domains are linked via a linker sequence, e.g., an oligopeptide of approximately 15 amino acids such as (Gly4Ser)3, as well as other art recognized linkers. Methods of making scFv molecules are known in the art. (see, e.g., Bird et al (1988) Science 240, 423; Huston et al (1988) Proc. Natl. Acad. Sci, USA 85, 5879; Gilliland et al. 1996. Tissue Antigens. 47:1; Winberg et al. 1996. Immunological Reviews 153:209; Hayden et al. 1996. Tissue Antigens. 48:242).

For example, VL and VH from a hybridoma of interest (e.g., a novel hybridoma made using methods described herein or known in the art or a hybridoma known to produce anti-CD28 antibodies (see, e.g., U.S. Pat. No. 5,948,893) can be cloned and expressed as a scFv protein. mRNA can be isolated from hybridoma cells producing anti-CD28 antibody. Typically, total RNA is isolated by extraction methods well known in the art, such as extraction with phenol at acid pH or extraction with guanidinium thiocyanate followed by centrifugation in cesium chloride solutions or using a commercially available kit (e.g., from Stratagene (Torrey Pines, Calif.). These procedures, and others for RNA extraction, are disclosed in J. Sambrook et al., “Molecular Cloning: A Laboratory Manual” (2d ed., Cold Spring Harbor Laboratory Press, Cold Spring Harbor, N.Y., 1989), ch. 7, “Extraction, Purification, and Analysis of Messenger RNA From Eukaryotic Cells,” pp. 7.1-7.25. Optionally, the mRNA can be isolated from the total mRNA by chromatography on oligo (dT) cellulose, but this step is not required.

To synthesize cDNA, primers complementary to the κ or λ light chain constant region and to the constant region of the heavy chain (e.g., γ2a) are preferably used to initiate synthesis. Amplification can be carried out by any procedure allowing high fidelity amplification without slippage. Preferably, amplification is by the polymerase chain reaction procedure (K. B. Mullis & F. A. Faloona, “Specific Synthesis of DNA in vitro Via a Polymerase-Catalyzed Chain Reaction,” Meth. Enzymol. 155:335-350 (1987); K. Mullis et al., “Specific Enzymatic Amplification of DNA in vitro: The Polymerase Chain Reaction,” Cold Spring Harbor Symp. Quant. Biol. 51:263-273 (1986); R. K. Saiki et al., “Primer-Directed Enzymatic Amplification of DNA with a Thermostable DNA Polymerase,” Science 238:487-491 (1988)).

One preferred procedure uses singlesided or anchored PCR (E. Y. Loh et al., “Polymerase Chain Reaction with Single-Sided Specificity: Analysis of T-cell Receptor 6 Chain,” Science 243:217-220 (1989)). This procedure uses homopolymer tailing of the 3′-end of the reverse transcript; PCR amplification is then performed with a specific 3′-primer and a second oligonucleotide consisting of a homopolymer tail complementary to the homopolymer tail added to the 3′-end of the transcript attached to a sequence with a convenient restriction site, termed the anchor. One version is described in (Y. L. Chiang et al., “Direct cDNA Cloning of the Rearranged Immunoglobulin Variable Region,” Biotechniques 7:360-366 (1989)).

The PCR products are cloned into a suitable host, e.g., E. coli. A number of cloning vectors suitable for cloning into E. coli are known and are described in vol. 1 of Sambrook et al., supra, Ch. 1, “Plasmid Vectors,” pp. 1.1-1.10. The exact manipulations required depend on the particular cloning vector chosen and on the particular restriction endonuclease sites used for cloning into the vector. One preferred vector is pUC19. For cloning into pUC19, the PCR products are treated with the Klenow fragment of E. coli DNA polymerase I and with the four deoxyribonucleoside triphosphates to obtain blunt ends by filling single-stranded regions at the end of the DNA chains. PCR can then be used to add Eco RI and Bam HI restriction sites to the 5′-end and 3′-ends, respectively, of the amplified fragment of cDNA of light-chain origin (the VL fragment). Similarly, Xba I and Hind III restriction sites are added to the amplified fragment of cDNA of heavy chain origin (the VH fragment). The fragments are digested with the appropriate restriction endonucleases and are cloned into pUC19 vector that had been digested with: (1) Eco R^{I} and Bam HI for VL and (2) Xba I and Hind III for VH. The resulting constructs can be used to transform a competent cell, e.g., an E. coli strain.

Clones containing VL and VH are preferably identified by DNA sequencing. A suitable DNA sequencing procedure is the Sanger dideoxynucleotide chain termination procedure. Such a procedure can be performed using the Sequenase 2.0 kit (United States Biochemical, Cleveland, Ohio), with forward and reverse primers that anneal to the pUC19 sequences flanking the polycloning site. Preferably, consensus sequences for VL and VH are determined by comparing the sequences of multiple clones and aligning the sequences with corresponding murine VL and VH variable region sequences (E. A. Kabat et al., “Sequences of Proteins of Immunological Interest” (4th ed., U.S. Department of Health and Human Services, Bethesda, Md., 1987)).

Clones containing VL and VH sequences can be placed in an expression cassette incorporating a single-chain antibody construct including the VL and VH sequences separated by a linker. The expression cassette can be constructed by overlap extension PCR in which the peptide linker between the VL and VH is encoded on the PCR primers. In one highly preferred procedure, the 5′-leader sequence is removed from VL and replaced with a sequence containing a Sal I site preceding residue 1 of the native protein. Constant region residues from the 3′-end are replaced with a primer adding a sequence complementary to a sequence coding for a linker sequence (e.g., the 16-residue linker sequence ESGSVSSEELAFRSLD [J. K. Batra et al., J. Biol. Chem. 265:15198-15202 (1990)] or [(Gly4Ser)3) Gilliland et al. 1996. Tissue Antigens 47:1].

For the VH sequence, a VH primer adds the “sense” sequence encoding the linker, e.g., the 16-residue linker sequence given above to the VH 5′-end preceding residue 1 of the mature protein and substitutes a sequence complementary to a Bcl I site for the constant region residues at the 3′-end.

The polymerase chain reaction can then be used with a mixture of VL and VH cDNA, as templates, and a mixture of the four primers (two linker primers and two primers containing restriction sites). This creates a single DNA fragment containing a VL-linker-VH sequence flanked by Sal I and Bcl I sites. The DNA construct is then preferably passaged through, e.g., E. coli cells. The passaged construct is then digested with Sal I and Bcl I.

For preparation and expression of the fusion protein, digested DNA from the preceding step is then ligated into a pCDM8 vector containing the anti-CD28 light chain leader sequence followed by a Sal I site and a Bcl I site preceding cDNA encoding, e.g, a human or humanized Ig tail (e.g., IgG) in which cysteines in the hinge region are mutated to serines to inhibit dimerization (P. S. Linsley et al., “Binding of the B Cell Activation Antigen B7 to CD28 Costimulates T-Cell Proliferation and Interleukin-2 mRNA Accumulation,” J. Exp. Med. 191:721-730 (1991) or another peptide molecule (Gilliland et al. 1996. Tissue Antigens 47:1).

The resulting construct is capable of expressing anti-CD28 scFv antibody. Exemplary constructs comprise non-human (e.g., murine) CDRs and human constant regions. The constructs can be placed in a vector, e.g., a plasmid.

Plasmid DNA can then isolated and purified, such as by cesium chloride density gradient centrifugation. The purified DNA is then transfected, e.g., into a prokaryotic cell or eukaryotic cell, using methods that are known in the art. A highly preferred cell line is monkey COS cells. A preferred method of introducing DNA is by DEAE-dextran, but other methods are known in the art. These methods include contacting a cell with coprecipitates of calcium phosphate and DNA, use of a polycation, polybrene, or electroporation. These methods are described in J. Sambrook et al., “Molecular Cloning: A Laboratory Manual,” supra, vol. 3, pp. 16.30-16.55.

Preferably, recombinant DNA containing the sequence coding for the fusion protein is expressed by transient expression, as described in A. Aruffo, “Transient Expression of Proteins Using COS Cells,” in Current Protocols in Molecular Biology (2d ed., F. M. Ausubel et al., eds., John Wiley & Sons, New York, 1991), pp. 16.13.1-16.13.7.

B. Other Agents

In addition to antibodies which bind to CD28, other agents known in the art can also be used to inhibit activation of CD28 and thus block CD28-mediated signaling. Any agent which binds to CD28 to effectively block ligand binding, without itself activating the CD28 receptor (e.g., via aggregation of the receptor) will effectively block CD28-mediated signaling. Alternatively, any agent which binds to a ligand(s) of CD28 to prevent binding and activation of CD28 will also block CD28-mediated signaling. A variety of such agents are know in the art.

Exemplary Agents

One such agent which will bind to CD28 without triggering activation is a soluble form of ligand which is in monomeric form. A soluble form of a CD28 ligand may contain an amino acid sequence corresponding to the extracellular domain of the ligand protein or any fragment thereof which does not include the cytoplasmic and/or transmembrane regions. Alternatively, the soluble form may contains a smaller region which is involved in CD28 binding. Such polypeptides, when produced recombinantly in a host cell, will be secreted freely into the medium, rather than anchored in the membrane. It is critical that the soluble form of the ligand be in monomeric form, so as not to cross link the CD28 molecule, and thus activate CD28-mediated signaling.

In one embodiment, the soluble ligand of CD28 is derived from a naturally occurring B7 molecule (e.g., B7-1, B7-2 or B7-3). DNA sequences encoding B7 proteins are known in the art, see e.g., B7-2 (Freeman et al. 1993 Science. 262:909 or GenBank Accession numbers P42081 or A48754); B7-1 (Freeman et al. J. Exp. Med. 1991. 174:625 or GenBank Accession numbers P33681 or A45803. The extracellular portion of the ligand (e.g., approximately amino acid residues 1-208 of the sequence of B7-1 or approximately amino acids 24-245 of the sequence of B7-2), or a fragment thereof which is sufficient for CD28 binding is used to generate the soluble ligand. It may further be useful to express the portion or fragment of the ligand as a fusion protein. Polypeptides having binding activity (e.g., binding to CD28) of a B7 molecule, and having a sequence which differs from a naturally occurring B7 molecule due to degeneracy in the genetic code, can also be expressed in soluble form and are also within the scope of the invention. Such polypeptides are functionally equivalent to B7, (e.g., a polypeptide having B7 activity) but differ in sequence from the sequence of B7 molecules known in the art. It will be appreciated by one skilled in the art that these variations in one or more nucleotides (up to about 3-4% of the nucleotides) of the nucleic acids encoding peptides having the activity of a novel B lymphocyte antigen may exist among individuals within a population due to natural allelic variation. Such nucleotide variations and resulting amino acid polymorphisms are also within the scope of the invention. Furthermore, there may be one or more isoforms or related, cross-reacting B7 molecules.

By way of example, to express a secreted (soluble) form of the B7-1 polypeptide comprising amino acids 1-212, a PCR product may be synthesized using the following two oligonucleotide primers and the B7-1 cDNA clone: (1) a sense primer consisting of a restriction enzyme site and 20 nucleotides corresponding to the translational initiation site and the first few amino acid codons of B7-1, and (2) an anti-sense primer consisting of 20 nucleotides corresponding to the last few amino acid codons of B7-1 ending at codon 212, (i.e., before the transmembrane region) followed by a stop codon and a restriction enzyme site. The PCR product may then be digested with the restriction endonuclease whose recognition sequence is in the PCR primers, gel purified, eluted, and ligated into an appropriate expression vector. The expression construct may then be introduced into a eukaryotic cell such as Cos7, where the B7-1 polypeptide fragment is synthesized and secreted. The B7-1 polypeptide fragment thus produced can then readily be obtained from the culture media. Such a soluble form of B7-1 was produced in U.S. Pat. No. 6,071,716, the contents of which are incorporated herein by reference.

Another exemplary agent which will bind to CD28 to block CD28-mediated signaling is a peptidomimetic or a small molecule.

Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference) and are usually developed with the aid of computerized molecular modeling. Peptide mimetics that are structurally similar to CD28, CD28 ligands, or functional variants thereof, can be used to produce an equivalent product to the blocking agents described above. Generally, peptidomimetics are structurally similar to the paradigm polypeptide but have one or more peptide linkages optionally replaced by a linkage selected from the group consisting of: —CH2NH—, —CH2S—, —CH2—CH2—, —CH═CH— (cis and trans), —COCH2—, —CH(OH)CH2—, and —CH2SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2—); Spatola, A. F. et al. (1986) Life Sci. 38:1243-1249 (—CH₂—S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2—); Jennings-White, C. et al. (1982) Tetrahedron Lett. 23:2533 (—COCH2—); Szelke, M. et al. European Appln. EP 45665 (1982) CA: 97:39405 (1982)(—CH(OH)CH2—); Holladay, M. W. et al. (1983) Tetrahedron Lett. (1983) 24:4401-4404 (—C(OH)CH2—); and Hruby, V. J. (1982) Life Sci. (1982) 31:189-199 (—CH2—S—); each of which is incorporated herein by reference. A particularly preferred non-peptide linkage is —CH2NH—. Such peptide mimetics may have significant advantages over polypeptides, including, for example: more economical production, greater chemical stability, enhanced pharmacological properties (half-life, absorption, potency, efficacy, etc.), altered specificity (e.g., a broad-spectrum of biological activities), and reduced antigenicity.

For example, peptidomimetics may be specifically designed from information about potential contact surfaces of the CD28 molecule with its ligand, or regions of the CD28 molecule responsible for mediating homodimer formation in order to disrupt the appropriate presentation of the homodimers. Such an approach was used by El Tayar et al., (WO 98/56401 (1998)), the contents of which are incorporated herein by reference, in the design of peptidomimetics which inhibit CD28 mediated signaling.

Derivatives of the present invention include polypeptides (e.g, anti-CD28 antibodies including binding fragments of anti-CD28 antibodies such as Fab, F(v), Fab′, F(ab′)₂, or single chain Fvs that have been fused with another compound, such as a compound to increase the half-life of the polypeptide and/or to reduce potential immunogenicity of the polypeptide (for example, polyethyleneglycol “PEG” and alpha-1-antitrypsin). In the case of PEGylation, the fusion of the polypeptide to PEG can be accomplished by any means known to one skilled in the art. For example, PEGylation can be accomplished by first introducing a cysteine mutation into the polypeptide, followed by site-specific derivatization with PEG-maleimide. The cysteine can be added to the C-terminus of the peptides. (See, for instance, Tsutsumi et al., Proc Natl Acad Sci USA 2000 Jul. 18; 97(15):8548-53).

The term “small molecule” is a term of art and included molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

A number of agents which bind to the CD28 ligand to prevent CD28 binding and thus block CD28-mediated signaling are known in the art. One such agent is a soluble form of CD28. A soluble form of CD28 is usually made of the extracellular portion of the receptor, or a fragment thereof which retains the ability to bind to the ligand. In one embodiment, the portion or fragment of the receptor is produced in the form of a fusion protein, e.g., an Ig fusion protein. One such soluble form of a CD28 molecule has been used to block the transduction of a costimulatory signal in a T cell (see e.g., U.S. Pat. No. 5,521,288).

In addition, a soluble form of a receptor which binds to a CD28 ligand (e.g., CTLA4 or ICOS) will also prevent ligand binding of CD28 to block CD28-mediated signaling. Such soluble forms of these cell surface molecules have been found to block the transduction of a costimulatory signal in a T cell. In one embodiment, a soluble form of a CD28 or ICOS molecule can be used to block the transduction of a costimulatory signal in a T cell (see e.g., U.S. Pat. No. 5,521,288).

In one embodiment, the agent which blocks CD28-mediated signaling is a soluble form of CTLA4. DNA sequences encoding the human and murine CTLA4 protein are known in the art, see e.g., Dariavich, et al. (1988) Eur. J. Immunol 18(12), 1901-1905; Brunet, J. F., et al. (1987) supra; Brunet, J. F. et al. (1988) Immunol Rev. 103:21-36; and Freeman, G. J., et al. (1992) J. Immunol. 149, 3795-3801. In certain embodiments, the soluble CTLA4 protein comprises the entire CTLA4 protein. In preferred embodiments, a soluble CTLA4 protein comprises the extracellular domain of a CTLA4 protein. For example, a soluble, recombinant form of the extracellular domain of CTLA4 has been expressed in yeast (Gerstmayer et al. 1997. FEBS Lett. 407:63). In other embodiments, the soluble CTLA4 proteins comprise at least a portion of the extracellular domain of CTLA4 protein which retains the ability to bind to B7-1 and/or B7-2.

In one embodiment the soluble CTLA4 protein or portion thereof is a fusion protein comprising at least a portion of CTLA4 which binds to B7-1 and/or B7-2 and at least a portion of a second non-CTLA4 protein. For example, a soluble, recombinant form of the extracellular domain of CTLA4 has been expressed in yeast (Gerstmayer et al. 1997. FEBS Lett. 407:63). In preferred embodiments, the CTLA4 fusion protein comprises a CTLA4 extracellular domain which is fused at the amino terminus to a signal peptide, e.g., from oncostatin M (see e.g., WO93/00431).

In a particularly preferred embodiment, a soluble form of CTLA4 is a fusion protein comprising the extracellular domain of CTLA4 fused to a portion of an immunoglobulin molecule. Such a fusion protein, CTLA4 μg, can be made using methods known in the art (see e.g., Linsley 1994. Perspectives in Drug Discovery and Design 2:221; Linsley WO 93/00431 and U.S. Pat. Nos. 5,770,197, and 5,844,095).

Antibodies which bind to a CD28 ligand to prevent CD28 binding also block CD28-mediated signaling. In one embodiment, antibodies for use in the instant methods bind to at least one B7 molecule. In yet another embodiment, an antibody of the invention binds to only one B7 molecule (e.g., to B7-1 and not to B7-2). Such antibodies are known in the art. For example, The 2D10 hybridoma, producing the 2D 10 antibody, has been described (Journal of Immunology. 1994. 152:2105). In addition, for use in combination with an anti-B7-2 antibody, several anti-B7-1 antibodies are known or are readily available (see, e.g., U.S. Pat. No. 5,869,050; Powers G. D., et al. (1994) Cell. Immunol 153, 298-311; Freedman, A. S. et al. (1987) J. Immunol. 137:3260-3267; Freeman, G. J. et al. (1989) J. Immunol 143:2714-2722; Freeman, G. J. et al. (1991) J. Exp. Med. 174:625-631; Freeman, G. J. et al. (1993) Science 262:909-911; WO 96/40915). Such antibodies are also commercially available, e.g., from R&D Systems (Minneapolis, Minn.) and from Research Diagnostics (Flanders, N.J.). Antibodies to B7-2 known in the art are, for example, anti-human B7-2 monoclonal antibodies produced by hybridomas HA3.1F9, HA5.2B7 and HF2.3D1. Monoclonal antibody HA3.1F9 is of the IgG1 isotype; monoclonal antibody HA5.2B7 is of the IgG2b isotype; and monoclonal antibody HF2.3D1 is of the IgG2a isotype. The preparation and characterization of these antibodies is described in detail in U.S. Pat. No. 6,084,067 (2000), the contents of which are incorporated herein by reference.

To generate antibodies to a ligand of CD28, such as a B7 protein (e.g., B7-1, B7-2 or B7-3) full-length B7 protein, or a peptide fragment thereof, having an amino acid sequence based on the predicted amino acid sequence of the B7 protein, anti-protein/anti-peptide polyclonal antisera or monoclonal antibodies can be made using standard methods, described above. A mammal, (e.g., a mouse, hamster, or rabbit) can be immunized with an immunogenic form of the protein or peptide which elicits an antibody response in the mammal. The immunogen can be, for example, a recombinant B7 protein, or fragment thereof, a synthetic peptide fragment or a cell that expresses a B lymphocyte antigen on its surface. The cell can be for example, a splenic B cell or a cell transfected with a nucleic acid molecule encoding a B lymphocyte antigen such that the B lymphocyte antigen is expressed on the cell surface. The immunogen can be modified to increase its immunogenicity. For example, techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay can be used with the immunogen as antigen to assess the levels of antibodies.

Screening Assays to Identify Novel Agents

A number of screening assays for identifying an agent (e.g., antibodies, peptides, peptidomimetics, small molecules or other drugs) that blocks CD28-mediated signaling are available in the art. Generally speaking, the agent is identified from one or more test agents (also referred to herein as candidate or test compounds) which are assayed for the ability block CD28-mediated signaling with a standard in vitro assay for immune response wherein CD28-mediated signaling, and thus the immune response, is downregulated by the presence of the functional agent. A number of suitable readouts of immune cell activation (e.g., cell proliferation or effector function such as antibody production, cytokine production, and phagocytosis) in the presence of the agent exist in the art. One commonly used assay is a T cell activation assay.

Typically, the chosen assay is manipulated by standard methods to induce an immune response via CD28-mediated signaling, in the presence or absence of a test agent. A comparative reduction in the CD28-mediated signaling, e.g., a reduction in the induction of the immune response, in the presence of the test agent indicates the test agent blocks CD28-mediated signaling. Inhibition of CD28-mediated signaling, as detected, e.g., by downregulation of the immune response results in a statistically significant and reproducible decrease in the immune response or downregulation of T cell activation preferably as measured by the assay. Agents that block CD28-mediated signaling can be identified by their ability to inhibit immune cell proliferation and/or effector function or to induce anergy when added to such an in vitro assay.

For example, immune cells are cultured in the presence of an agent that stimulates signal transduction via CD28. A readout of cell activation can be employed to measure cell proliferation or effector function (e.g., antibody production, cytokine production, phagocytosis) in the presence of the activating agent, a number of such readouts are known in the art. The ability of an agent to block cell activation can be readily determined by measuring the ability of the agent to affect a decrease in proliferation or effector function. A method for the identification of such agents is discussed in more detail below.

The test compound of the present invention can be, for instance, any of the compounds described above. In one embodiment, the compound is an agent not previously known to inhibit CD-28-mediated signaling. In another embodiment, a plurality of compounds are tested. Such compounds may be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In another embodiment, an assay is a cell-based assay comprising contacting a cell expressing a CD28 with a test compound and determining the ability of the test compound to inhibit or block the activity of CD28 with respect to induced signaling. Determining the ability of the test compound to block CD28 induced signaling can be accomplished, for example, by determining the ability of CD28 to bind to or interact with its natural ligands. Determining the ability of CD28 to bind to or interact with its natural ligand can be accomplished, for instance by measuring direct binding, or by detection of CD28-mediated signaling.

In a direct binding assay, the CD28 protein, or a modified version or mimetic thereof (or their respective receptors) can be coupled with a radioisotope or enzymatic label such that binding of the CD28 protein to a target molecule can be determined by detecting the labeled protein in a complex. For example, CD28 molecules, can be labeled with ¹²⁵I, ³⁵S, ¹⁴C, or ³H, either directly or indirectly, and the radioisotope detected by direct counting of radioemmission or by scintillation counting. Alternatively, CD28 molecules can be enzymatically labeled with, for example, horseradish peroxidase, alkaline phosphatase, or luciferase, and the enzymatic label detected by determination of conversion of an appropriate substrate to product.

A test agent or compound may function to block CD28-mediated signaling by inhibiting the interaction between CD28 and its ligand. Such an activity of a test agent or compound to modulate the interaction between CD28 and its ligand can be determined without the labeling of any of the interactants. For example, a microphysiometer can be used to detect the interaction of CD28 with its ligand without the labeling of either CD28 or the ligand (McConnell, H. M. et al. (1992) Science 257: 1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, determining the ability of a test agent to block CD28-mediated signaling can be accomplished by determining the activity of a ligand of CD28 at inducing signaling via CD28 in the presence of the test agent. CD28-mediated signaling can be determined, for instance, by detecting induction of a cellular second messenger (e.g., tyrosine kinase activity), detecting catalytic/enzymatic activity of an appropriate substrate, detecting the induction of a reporter gene (comprising a target-responsive regulatory element operatively linked to a nucleic acid encoding a detectable marker, e.g., chloramphenicol acetyl transferase), or detecting another cellular response regulated by CD28.

In another embodiment, the assay is a cell-free assay in which a CD28 molecule is contacted with a test agent and the ability of the test agent to inhibit the activity of a CD28 ligand or biologically active portion thereof (at inducing signaling via CD28) is determined. This can be accomplished, for example, by determining the ability of the ligand to bind CD28, e.g., using a technology such as real-time Biomolecular Interaction Analysis (BIA) (Sjolander, S, and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

III. Pharmaceutical Compositions

The active molecules of the invention (e.g., antigen binding portions of anti-CD28 antibodies or small molecules) can be suspended in a any known physiologically compatible pharmaceutical carrier, such as cell culture medium, physiological saline, phosphate-buffered saline, or the like, to form a physiologically acceptable, aqueous pharmaceutical composition. Parenteral vehicles include sodium chloride solution, Ringer's dextrose, dextrose and sodium chloride, and lactated Ringer's. Other substances may be added as desired such as antimicrobials.

An active molecule for donwmodulating the immune response can be incorporated into a composition, e.g., a pharmaceutical composition suitable for administration. Such compositions typically further comprise a carrier, e.g., a pharmaceutically acceptable carrier. As used herein the language “carrier” is intended to include any and all solvents, dispersion media, coatings, antibacterial and antifungal agents, isotonic and absorption delaying agents, and the like, compatible for use with cells, e.g., compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions can be included in a container, pack, or dispenser together with instructions for administration. The kit can further comprise a means for administering the active molecule of the invention, e.g., one or more syringes. The kit can come packaged with instructions for use.

IV. Uses and Methods of the Invention

The active molecules of the invention are useful in downmodulating the immune response. The present invention provides for both prophylactic and therapeutic methods of treating a subject at risk of (or susceptible to) a disorder or having a disorder associated with an aberrant or undesirable immune response, e.g., autoimmune diseases, allergy and allergic reactions, transplantation rejection, and established graft versus host disease in a subject.

The active molecules of the invention can be used to downmodulate both primary and secondary immune responses. They can be used to downmodulate immune responses mediated, either directly or indirectly (e.g., based on helper function) by T cells. In one embodiment, the subject compositions and methods are used to downmodulate CD4+ T cell responses. In another embodiment, the subject compositions and methods are used to downmodulate CD8+ T cell responses.

In one aspect, the invention provides a method for preventing an undesirable immune response in a subject. Administration of an active molecule of the invention can occur prior to the manifestation of symptoms for which modulation of the immune response would be beneficial, such that a disease or disorder is prevented or, alternatively, delayed in its progression. Such administration can be used to prevent or downmodulate primary immune responses. Another aspect of the invention pertains to methods of modulating an immune response for therapeutic purposes, e.g., to downmodulate ongoing or secondary immune responses.

The present invention provides methods of treating a subject afflicted with a disease or disorder that would benefit from downmodulation of the immune response by contacting cells from the subject with an agent that specifically binds to CD28. An agent that specifically binds to CD28 can be administered ex vivo (e.g., by contacting the cell with the agent in vitro) or, alternatively, in vivo (e.g., by administering the agent to a subject). Likewise, a cell can be made to express an agent that specifically binds to CD28 either in vivo or ex vivo.

Downmodulation of the immune response is useful to downmodulate the immune response, e.g., in situations of tissue, skin and organ transplantation, in graft-versus-host disease (GVHD), allergy, or in autoimmune diseases. Autoimmune diseases that will benefit from the instant methods include those mediated by humoral and/or cellular mechanisms. Exemplary autoimmune diseases or disorders include, but are not limited to: systemic lupus erythematosus, diabetes mellitus (e.g., autoimmune diabetes or type I diabetes), rheumatoid arthritis, multiple sclerosis, myasthenia gravis, systemic lupus enthmatosis, and autoimmune thyroiditis, vitiligo, alopecia, celiac disease, inflammatory bowel disease, chronic active hepatitis, Addison's disease, Hashimoto's disease, Graves disease, atrophic gastritis/pernicious anemia, acquired hypogonadism/infertility, hypoparathyroidism, Myasthenia gravis, Coombs positive hemolytic anemia, chronic allergic diseases (such as asthma, hay fever, or allergic rhinitis), and Sjogren's syndrome.

For example, blockage of immune responses results in reduced tissue destruction in tissue transplantation. Typically, in tissue transplants, rejection of the transplant is initiated through its recognition as foreign by immune cells, followed by an immune reaction that destroys the transplant. The administration of an active molecule of the invention prior to or at the time of transplantation, can inhibit the immune response. In one embodiment, a cell for transplantation is caused to express a soluble form of an agent that specifically binds to CD28.

In one embodiment, use of the active molecules of the invention is sufficient to anergize the immune cells, thereby inducing tolerance in a subject. In another embodiment, the active molecules of the invention are administered repeatedly (i.e., more than once) to achieve optimal reduction in one or more immune response(s). In one embodiment, long term tolerance is induced in a subject and may avoid the necessity of repeated administration of these blocking reagents.

To achieve sufficient immunosuppression or tolerance in a subject, it may also be desirable to block the costimulatory function of other molecules. For example, it may be desirable to block the function of B7-1, B7-2, or B7-1 and B7-2 by administering a soluble form of a combination of peptides having an activity of each of these antigens or blocking antibodies against these antigens (separately or together in a single composition) prior to or at the time of transplantation. Other downmodulatory agents that can be used in connection with the downmodulatory methods of the invention include, for example, blocking antibodies against other immune cell markers or soluble forms of other receptor ligand pairs (e.g., agents that disrupt the interaction between CD40 and CD40 ligand (e.g., anti CD40 ligand antibodies)), antibodies against cytokines, fusion proteins (e.g., CTLA4-Fc), and/or immunosuppressive drugs, (e.g., rapamycin, cyclosporine A or FK506).

The active molecules of the invention are also useful in treating autoimmune disease. Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. The active molecules of the invention are useful to inhibit immune cell activation and prevent production of autoantibodies or cytokines which may be involved in the disease process.

Inhibition of immune cell activation can also be used therapeutically in the treatment of allergy and allergic reactions, e.g., by inhibiting IgE production. An active molecule of the invention can be administered to an allergic subject to inhibit immune cell mediated allergic responses in the subject. Administration of an active compound can be accompanied by exposure to allergen. Allergic reactions can be systemic or local in nature, depending on the route of entry of the allergen and the pattern of deposition of IgE on mast cells or basophils. Thus, inhibition of immune cell mediated allergic responses can be effected locally or systemically by administration of an active molecule of the invention.

The invention also includes methods of treating a transplant recipient, preventing transplant rejection, or prolonging graft survival in a transplant recipient by administering to the recipient an effective amount of a non-activating anti-CD28 antibody. Prolonging graft survival as used herein refers to any increase in graft acceptance by the recipient (e.g., about 1 day, 5 days, 10 days, 50 days, 100 days, or more).

The prevention and/or treatment of graft rejection contemplated by the present invention includes transplantation of organs or tissues from HLA matched and unmatched allogeneic human donors, or xenografts from donors of other species. Such transplanted grafts include hearts, lungs, kidneys, livers, skin and other organs or tissues transplanted from donor to recipient. To ensure successful organ transplantation, it is desirable to obtain the graft from the patient's identical twin or his/her immediate family member. This is because organ transplants evoke a variety of immune responses in the host, which results in rejection of the graft and graft-versus-host disease (hereinafter, referred to as “GVHD”).

A non-activating anti-CD28 antibody may also be used to treat transplant recipients with various forms of GVHD including acute and chronic GVHD that is either naive or refractory to immunosuppressive treatment. A non-activating anti-CD28 antibody may also be used as prophylaxis to prevent onset of GVHD by pretreating the transplant recipient prior to the transplantation and/or treating the recipient within a certain time window post transplantation.

In one embodiment, a method is provided for prolonging graft survival in a subject. The method comprises administering to the transplant recipient a composition including a non-activating anti-CD28 antibody. Dosage amounts and frequency will vary according to the particular non-activating anti-CD28 antibody, the dosage form, and individual patient characteristics. Generally speaking, determining the dosage amount and frequency for a particular non-activating CD28 antibody, dosage form, and individual patient characteristic can be accomplished using conventional dosing studies, coupled with appropriate diagnostics. In certain embodiments, the dosage frequency ranges from daily to weekly to monthly doses.

In certain embodiments, the non-activating anti-CD28 antibody is administered in an amount between about 1 mg/kg and 100 mg/kg. In certain embodiment, the non-activating anti-CD28 antibody is administered in an amount between about 1 mg/kg and 50 mg/kg. In further embodiments, the non-activating anti-CD28 antibody is administered in an amount between about 1 mg/kg and 25 mg/kg. In still further embodiments, the non-activating anti-CD28 antibody is administered in an amount between about 1 mg/kg and 10 mg/kg. In still further embodiments, the non-activating anti-CD28 antibody is administered in an amount between about 1 mg/kg and 5 mg/kg. In one embodiment, the non-activating anti-CD28 antibody is administered in an amount between about 2 mg/kg.

The non-activating anti-CD28 antibody can be administered on the day the recipient receives the transplantation (e.g., in an amount between about 1 mg/kg and about 25 mg/kg), and can also be administered periodically (e.g., daily, weekly or monthly) after the recipient receives the transplantation (e.g., in an amount between about 1 mg/kg and about 5 mg/kg).

In one embodiment, a method is provided for treating a subject suffering from GVHD. The method comprises administering to the GVHD patient a composition including a non-activating anti-CD28 antibody. In one embodiment, a subject with steroid-refractory a GVHD is treated with a non-activating anti-CD28 antibody. The subject may additionally be treated with immunosuppressive agents not including the any immunosuppressive treatment previously administered to the subject.

A non-activating anti-CD28 antibody can also be used as a prophylaxis to prevent onset of GVHD or to reduce the effects of GVHD. A non-activating anti-CD28 antibody may be administered as a GVHD prophylaxis parenterally or orally to a transplant recipient within a predetermined time window before or after the transplantation.

A non-activating anti-CD28 antibody may also be used in combination with an immunosuppressive agent to prolong graft survival and/or prevent GVHD. The combination therapy may have any increase in the therapeutic effect including additive and synergistic therapeutic effects on the patients. A combination therapy may lower the amount of a non-activating anti-CD28 antibody and/or the other agent used in conjunction to achieve satisfactory therapeutic efficacy. As a result, potential side effects associated with high dose of drugs, such as myelosuppression, are reduced and the patient's quality of life is improved.

Various other therapeutic and immunosuppressive agents may be combined with a non-activating anti-CD28 antibody to prolong graft survival and/or to treat or prevent GVHD. The other therapeutic agents include, but are not limited to, immunosuppressive agents such as calcineurin inhibitors (e.g., cyclosporin A or FK506), steroids (e.g., methyl prednisone or prednisone), or immunosuppressive agents that arrest the growth of immune cells(e.g., rapamycin), anti-CD40 pathway inhibitors (e.g., anti-CD40 antibodies, anti-CD40 ligand antibodies and small molecule inhibitors of the CD40 pathway), transplant salvage pathway inhibitors (e.g., mycophenolate mofetil (MMF)), IL-2 receptor antagonists (e.g., Zeonpax© from Hoffmann-1a Roche Inc., and Simulet from Novartis, Inc.), or analogs thereof, cyclophosphamide, thalidomide, azathioprine, monoclonal antibodies (e.g., Daclizumab (anti-interleukin (IL)-2), Infliximab (anti-tumor necrosis factor), MEDI-205 (anti-CD2), abx-cb1 (anti-CD147)), and polyclonal antibodies (e.g., ATG (anti-thymocyte globulin)).

In yet another aspect, the invention relates to a method of ex vivo or in vitro treatment of blood derived cells, bone marrow transplants, or other organ transplants. The method comprises treating the blood derived cells, bone marrow transplants, or other organ transplants with a non-activating anti-CD28 antibody in an effective amount such that activities of T-lymphocytes therein are substantially inhibited, preferably by at least 50% reduction in activity, more preferably by at least 80% reduction in activity, and most preferably by at least 90% reduction in activity.

The invention is practiced in an in vitro or ex vivo environment. In a particular embodiment, practice of an in vitro or ex vivo embodiment of the invention might be useful in the practice of immune system transplants, such as bone marrow transplants or peripheral stem cell procurement. In such procedures, the non-activating anti-CD28 antibody might be used, as generally described above, to treat the transplant material to inactivate T-lymphocytes therein so that the T-lymphocyte mediated immune response is suppressed upon transplantation.

For example, the non-activating anti-CD28 antibody may be added to a preservation solution for an organ transplant in an amount sufficient to inhibit activity of T-lymphocytes of the organ. Such a preservation solution may be suitable for preservation of different kind of organs such as heart, kidney and liver as well as tissue therefrom. An example of commercially available preservation solutions is Plegisol (Abbott), and other preservation solutions named in respect of its origins include the UW-solution (University of Wisconsin), the Stanford solution and the Modified Collins solution (J. Heart Transplant (1988) Vol. 7(6):456 4467). The preservation solution may also contain conventional co-solvents, excipients, stabilizing agents and/or buffering agents.

The dosage form of the non-activating anti-CD28 antibody may be a liquid solution ready for use or intended for dilution with a preservation solution. Alternatively, the dosage form may be lyophilized or power filled prior to reconstitution with a preservation solution. The lyophilized substance may contain, if suitable, conventional excipients.

The preservation solution or buffer containing a non-activating anti-CD28 antibody may also be used to wash or rinse an organ transplant prior to transplantation or storage. For example, a preservation solution containing a non-activating anti-CD28 antibody may be used to flush perfuse an isolated heart which is then stored at 4° C. in the preservation solution.

In another embodiment, practice of the invention might be used to condition organ transplants prior to transplantation. Prior to transplantation a non-activating anti-CD28 antibody may be added to the washing buffer to rid the transplant of active T-lymphocytes. The concentration of the non-activating anti-CD28 antibody in the preservation solution or wash buffer may vary according to the type of transplant. Other applications in vitro or ex vivo using A non-activating anti-CD28 antibody will occur to one of skill in the art and are therefore contemplated as being within the scope of the invention.

VI. Administration of Active Molecules of the Invention

The active molecules of the invention may be introduced into the subject to be treated by using one of a number of methods of administration of therapeutics known in the art. For example, active molecules may be administered parenterally (including, for example, intravenous, intraperitoneal, intramuscular, intradermal, and subcutaneous), by ingestion, or applied to mucosal surfaces. Alternatively, the active molecules of the invention are administered locally by direct injection at the site of an ongoing immune response.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELM (BASF, Parsippany, N.J.) or phosphate buffered saline (PBS). In all cases, the composition will be sterile and should be fluid to the extent that easy syringability exists. A composition will be stable under the conditions of manufacture and storage and are preferably preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Active molecules of the invention can be introduced into a subject with an antigen or antigens corresponding to those to which an immune response to be downmodulated is directed. Such molecules can be introduced into a subject prior to onset of an immune response or when an immune response is ongoing.

A “therapeutically effective amount” of a composition of the invention is a dose sufficient to reduce or suppress an immune response to the selected antigen.

Routes of administration include epidermal administration including subcutaneous or intradermal injections. Transdermal transmission including iontophoresis may be used, for example “patches” that deliver product continuously over periods of time.

Mucosal administration of the active molecules of the invention is also provided for, including intranasal administration with inhalation of aerosol suspensions. Suppositories and topical preparations may also be used. The dosage of a sufficient amount or number of the active molecules to downmodulate T response(s) in a subject can be readily determined by one of ordinary skill in the art. The active molecules may be introduced in at least one dose and either in that one dose or through cumulative doses are effective in reducing an immune response. The active molecules are administered in a single infusion or in multiple, sequential infusions.

Different subjects are expected to vary in responsiveness to such treatment. Dosages will vary depending on such factors as the individual's age, weight, height, sex, general medical condition, previous medical history, and immune status. Therefore, the amount or number of active molecules infused as well as the number and timing of subsequent infusions, is determined by a medical professional carrying out the therapy based on the response of the patient.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

After administration, the efficacy of the therapy can be assessed by a number of methods, such as assays that measure T cell proliferation, T cell cytotoxicity, antibody production, and/or clinical response. An decrease in the production of antibodies or immune cells recognizing the selected antigen will indicate a downmodulated immune response. Efficacy may also be indicated by improvement in or resolution of the disease (pathologic effects), associated with the reduction or disappearance of the unwanted immune response, or improvement in or resolution of the disease (pathologic effects) associated with the unwanted immune response (e.g. autoimmune disease) allergic reaction or transplant rejection). For example, standard methodologies can be used to assay, e.g., T cell proliferation, cytokine production, numbers of activated T cells, antibody production, or delayed type hypersensitivity. In addition or alternatively, improvement in a specific condition for which treatment is being given can be monitored, e.g., insulin levels can be monitored in a subject being treated for diabetes.

The practice of the present invention employs conventional techniques of molecular biology, microbiology, recombinant DNA, and immunology, within the skill of these arts. Such techniques are found in the scientific literature (See, e.g., Brock, Biology of Microorganisms, Eighth Ed., (1997), (Madigan et al., eds.), Prentice Hall, Upper Saddle River, N.J.; Sambrook et al., Molecular Cloning: A Laboratory Manual, Second Ed., (1989); Oligonucleotide Synthesis, M.J. Gait Ed., 1984, Animal Cell Culture, Freshney, ed., 1987; Methods in Enzymology, series, Academic Press, Inc.; Gene Transfer Vectors for Mammalian Cells, Miller and Calos, Eds., 1987; Handbook of Experimental Immunology, Weir and Blackwell, Eds., Current Protocols in Molecular Biology. Ausubel et al, Eds., 1987, and Current Protocols in Immunology, Coligan et al., Eds., 1991). These references are incorporated in their entirety herein by reference.

This invention is further illustrated by the following examples which should not be construed as limiting. The contents of all references, patents and published patent applications cited throughout this application, as well as the Figures and the Sequence Listing are incorporated herein by reference.

### EXAMPLES

The NOD mouse model for diabetes was used in Examples 1-5. The NOD mouse undergoes an autoimmune destruction of pancreatic islet B cells similar to that seen in patients with human type I diabetes. Infiltration of CD4+ and CD8+ T cells into the Islets of Langerhans begins at 4-5 weeks of age. Examples 1-5 show that in contrast to whole anti-CD28 antibody, PV1-scFv surprisingly prevents disease onset in both weanling NOD as well as adult female NOD mice.

### Example 1

### Anti-CD28 and PV1 (anti-CD28) scFv Bind to CD28 Equally

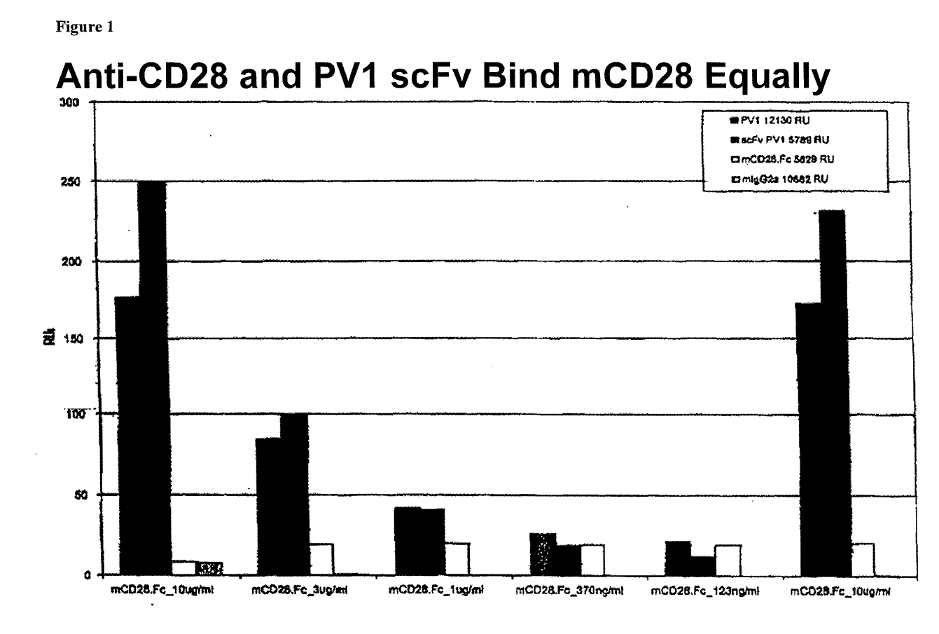

BIAcore experiments were performed which show that PV 1 scFv and anti-CD28 (PV1.10.17) bind equally well to murine CD28 (FIG. 1).

### Example 2

### PV1 (anti-CD28) scFv Inhibits T Cell Responses In Vitro

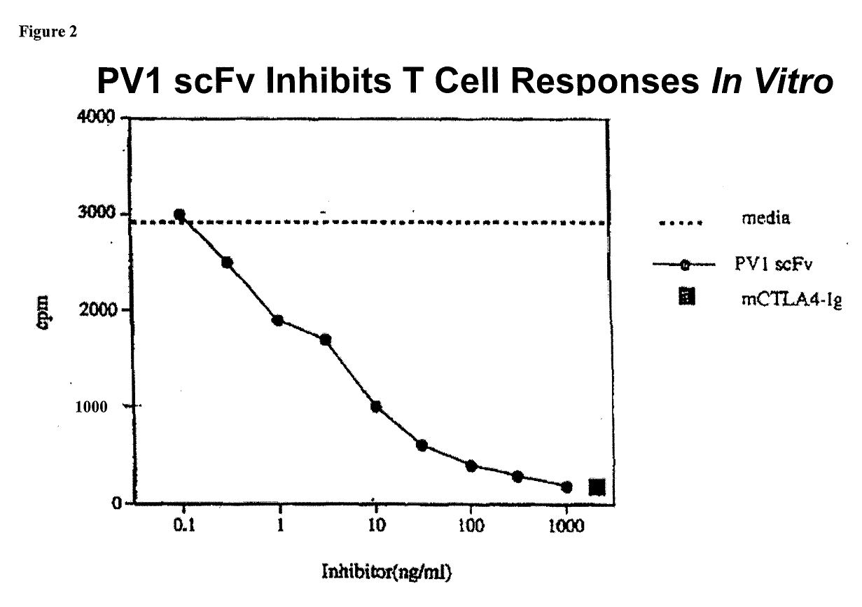

PV1-scFv blocks costimulation of anti-CD3 responses in vitro (FIG. 2). In this example, 1×10⁵ NOD spleen cells were cultured with 1 μg/ml anti-CD3. PV1 scFv or mCTLA4-Ig were added on day 0. Proliferation (cpm of ³H-thymidine incorporated into the DNA of the cells) was measured on day 3.

### Example 3

### PV1 (anti-CD28) Delays Disease Onset in Two Week Old NOD Female Mice

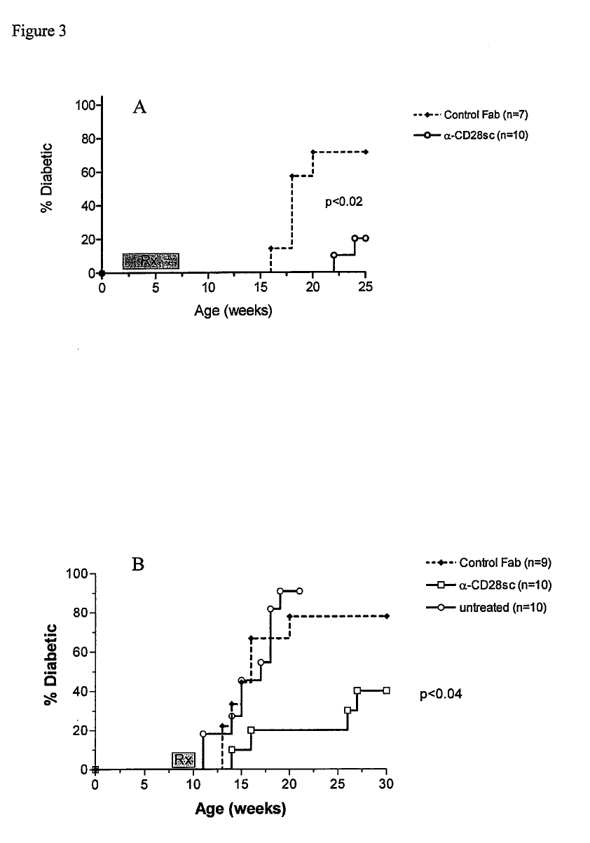

Two to three week old female NOD mice were injected with 50 pg PV1 scFv every other day for two weeks with an additional dose at five, six, and seven weeks. At 27 weeks of age, only 20% of the PV 1 scFv treated mice were diabetic, in contrast, 80% of control mice were diabetic (FIG. 3). In this example, 50 pg PV1 scFv or 710-Fab, was administered to 2 week old female NOD mice every other day for 14 days with an additional dose at 5, 6, and 7 weeks.

### Example 4

### PV1 scFv Delays Disease Onset in Adult (8 week old) NOD Female Mice

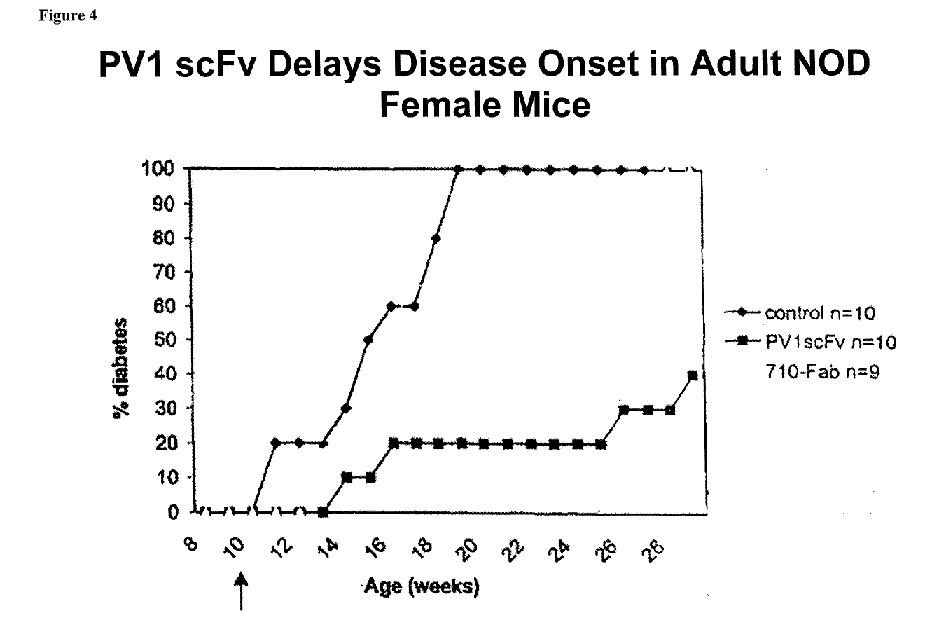

Adult female NOD mice were injected with 50 μg PV1 scFv daily from eight to ten weeks. At thirty weeks of age, only 40% of the PV1-scFv treated mice were diabetic, in contrast, 100% of control mice were diabetic (FIG. 4). In this example, 8 week old female NOD mice were injected with 50 μg of PV1scFv or control antibody daily for 14 days.

### Example 5

### Further Studies Showing Specific Blockade of CD28 can Prevent the Initiation and Progression of Diabetes in the NOD Mouse

Activation of T cells is an integral part of the pathogenesis of autoimmune diabetes in the NOD mouse. T cell activation is well documented to depend upon two separable signals delivered by Antigen Presenting Cells (APC). Islet antigens, presented by the unique I-A^{g7} molecule, activate autoreactive T cell receptors. A second signal, delivered by interaction of the T cell surface antigen, CD28, with B7 molecules, present on APC, promotes the expansion and survival of pathogenic T cells, which will eventually destroy insulin-producing B cells in Islets of Langerhans (Castano, L., and G. S. Eisenbarth (1990) Ann. Rev. Immunol. 6:647-679; Lenschow et al. (1996) Annu. Rv. Immunol. 14:233-2581). A second cell surface molecule, induced on activated T cells, CTLA4, also interacts with B7 (Linsley et al. (1991) J. Exp. Med. 174:561-5693). B7/CTLA4 interaction provides a down-regulatory signal to an activated T cell, apparently by counteracting intracellular signals, delivered through the T cell receptor. As such, T cell interaction with B7 can produce positive or negative effects on T cell activity, depending on the context of the interaction (Boussiotis et al. (1993) J. Exp. Med. 178:1753-1763; Freeman et al. (1993) J. Exp. Med. 178:2185-2192).

Reagents, which specifically target the B7 molecules, present on APC, have yielded complex results. Lenschow et al. ((1995) J. Exp. Med. 181:1145-1155) reported that treatment of weanling NOD mice with human CTLA4-Ig resulted in prevention of Immune Mediated Diabetes (IMD), indicating that B7-mediated signals are necessary for disease progression. Lenschow et al. further demonstrated that administration of antibodies to B7-2 prevented disease onset in weanling NOD mice. In contrast, treatment of NOD mice with antibodies to B7-1 resulted in exacerbation of diabetes. Finally, the combination of anti-B7-1 and anti-B7-2 exacerbated diabetes in the NOD mouse, consistent with the later finding of exacerbation of disease observed in B7-1/B7-2 double knockout mice (Salomon et al. (2000) Immunity 12:431-440).

Signaling through the CD28 molecule plays an active role in the pathogenesis of diabetes in the NOD model (Lenschow et al. (1996) Annu. Rv. Immunol. 14:233-258). Although the absence of CD28 on non-autoimmune strains of mice leads to decreased or absent immune responses (Green et al. (1994) Immunity 1:501-508), NOD mice made deficient in CD28 expression show enhanced diabetes onset (Salomon et al. (2000) Immunity 12:431-440). Arreaza et al. demonstrated that signaling through CD28 prevents diabetes onset when antibody to CD28 is injected into two to four week old NOD mice (Arreaza et al. (1997) J. Clin. Invest. 100:2243-2253). The mechanism of this protection was shown to be IL-4 dependent when antibody to IL-4 caused a return to the diabetes-prone phenotype. Injection of agonistic antibody to CD28 into mice from five to seven weeks of age did not prevent diabetes onset. Taken together, these data indicate an active role for CD28 signaling in diabetes onset in the NOD model.

In an attempt to clarify the role of B7-CD28 interaction in the NOD model, experiments were conducted directly targeting CD28. Using intact, agonistic anti-CD28 antibody, adult female and male NOD mice were treated and an acceleration of diabetes onset was observed in both groups of mice. In a second set of experiments, a single chain Fv fragment of the anti-murine CD28 antibody, PV1 was constructed and expressed. Construction of the scFv fragment, produced a monomeric reagent that is incapable of crosslinking CD28, and thus blocks CD28 signals. The purified scFv was able to inhibit B7-dependent proliferation and cytokine production in vitro. When injected in vivo, anti-CD28 scFv was able to prevent diabetes onset, when used either in weanling or adult animals. Histologic examination of the two treated groups yielded distinct results. Weanling animals, treated with anti-CD28 scFv, were protected from diabetes onset and demonstrated little or no islet infiltrates. Adult (eight week-old) mice were also protected from disease onset but had a remarkable peri-islet inflammation. These data specifically define the role of CD28 in NOD diabetes and indicate that a costimulation-dependent event may mediate the progression from inflammatory to destructive insulitis.

Materials and Methods

Animals

NOD/LtJ and NOD-scid mice were purchased from The Jackson Laboratory (Bar Harbor, Me.). Three-week old NOD/LtJ mice used herein were bred at Wyeth Research from stock originally purchased from The Jackson Laboratory. Female NOD mice housed in the Laboratory Animal Resources facility at Wyeth Research develop diabetes at approximately a 90% incidence by 30 weeks of age. Animals used herein were maintained in accordance with the guidelines of the Committee on Care and Use of Laboratory Animals of the Institute of Laboratory Animal Resources, National Research Council (Department of Health and Human Services Publication 85-23, revised in 1985).

Cell Lines

PV1.17.10 (anti-murine CD28) was obtained from Dr. Carl H. June, Naval Medical Research Institute, Bethesda, Md. H28.710 (anti-murine TCRx) was obtained from Dr. Ralph Kubo, National Jewish Center for Immunology and Respiratory Medicine, Denver, Colo. Both cell lines were maintained in vitro, at 370C, in media containing, RPMI 1640, 10% FCS, 1%, 1-glutamine, Sodium Pyruvate, HEPES, 5×10⁻⁵ M β-mercaptoethanol.

Generation of Anti-CD28 Single Chain Fv Construct

Whole cell RNA, derived from PV1.17.10 cells, was isolated by Guanidinium/CsCl cushions, then poly A⁺ selected using PolyAtract kit for mRNA (Promega,). 3.5 μg of polyA⁺ mRNA was used to construct an oligo-dT primed cDNA library, using a Zap Express kit (Stratagene). 150,000 plaques were screened using labeled oligonucleotides from constant regions of both heavy and light chains. Six double positives of each oligo probe were selected for second round screening. The two largest inserts, as determined by gel electrophoresis, from each chain were sequenced. One of each of the chains was full length.

Two PCR experiments were set up to amplify each chain. For the light chain, the sense primer (GACCGGAGGTCGACATGGATTCACAGATCCAGGTCCTCATG) was designed with 8 extra bases, a SalI site, and 27 bases corresponding to the kappa leader sequence. The anti-sense primer, AAATTTGGATCCGCCACCTCCGCGTCTTATC TCCAGCTTGGTGCCATC, contained 6 extra bases, a BamH1 site, sequence encoding GGGGS linker, and 26 bases of the J kappa region. For the heavy chain, the sense primer, AAATTTGGATCCGGAGGCGGAGGTTCTGGCGGAGGTGGGAGTGGCGG CCGCCAGGTCCAGTTGAAGCAGTCTGG, entailed 6 extra bases, sequence encoding GGGGSGGGGS linker, NotI site, and 24 bases of the V heavy leader region. The anti-sense primer, AAATTTTCTAGATCATCAGTGGTGGTGGTGGTGGTGGCTTCC GGTTCCTGAGGAGACGGTGACCTGGGT contained 6 extra bases, an XbaI site, two encoded stop sites, HIS6 region, a GTGS spacer sequence, and 21 bases of heavy chain J region.

Each chain was PCR amplified with 1 μg of template for 7 cycles (standard nucleotide and primer amounts) followed by a 10 minute 72 degree extension. The amplified bands were gel purified and digested with restriction endonucleases. The heavy chain ends were BamHI and XbaI cut. The light chain ends were BamHI and SalI cut. After digests, the fragments were purified using Qiaquick columns (Qiagen), and combined to ligate with expression construct pEDdc (SalI & XbaI cut). The insert of the finished construct was sequenced from both strands to confirm appropriate ligation.

Protein Purification

Anti-CD28 scFv protein was purified from CHO cell lines, expressing the scFv construct. Supernatant from the cell line was passed over a Ni column, eluted with an imidazole gradient, buffer exchanged and sterile filtered. Reduced and unreduced anti-CD28 scFv ran at approximately 28 Kd on polyacrylamide gels. Size exclusion chromatography indicated that the purified protein was present as a monomer. Fab fragments from H28.710 were prepared commercially, by papain cleavage and size exclusion purification (Maine Biotechnology, Portland, Me.). Fab fragments from H28.710, a hamster IgG that recognizes TCRα chain by western blotting, but does not bind cell surface TCRα, were prepared for use as a protein control using standard methods (Kubo et al. (1989) J Immunol 142:2736-2742).

Histological Analysis

Pancreas from sacrificed mice, were fixed in PBS containing 2% paraformaldehyde. Tissues were processed, sectioned and stained with Hematoxylin and Eosin by Pathology Associates International (Frederick, Md.). Masked slides from treated mice were scored using a standard scale for insulitis. Briefly, O— no islet infiltrate observed, 1—peri-islet infiltrates or less than 25% of the islet demonstrated cellular infiltrates, 2—greater than 25% but less than 50% of the islet was infiltrated, 3—more that 50% but less than 75% was infiltrated, 4—more than 75% of the islet mass was infiltrated by lymphocytes.

Polyclonal T Cell Activation

Spleen cells from NOD mice were activated using anti-CD3ε (145 2C11, PharMingen, San Diego, Calif.), at a concentration of 0.1-10 μg/ml. Cells were cultured in 96 well round bottom plates (Costar, Cambridge, Mass.). at 37° C. For IL-2 and IFN-γ measurements, supernatants were collected at 48 hours of culture, by aspirating 100 μl of media from well. 100 μl of fresh media was replaced at that time. Proliferation was measured by adding 0.5 mCi ³H-Thymidine (NEN, Cambridge, Mass.) to these cultures, then incubating an additional 24 hours. Cells were harvested on a Tomtec harvester (Wallac, Inc., Gaithersburg, Md.) and ³H-Thymidine incorporation was counted in an LSC counter (Wallac Microbeta).

Cytokine ELISA

Supernatants were assayed for cytokines by sandwich ELISA, using paired antibodies obtained from PharMingen. Immulon II plates (Dynatech, Chantilly, Va.) were coated overnight with 5 μg/ml purified anti-IL-2 or anti-IFNγ, as appropriate. Plates were blocked for 2 hours with PBS/0.5% Casein at 37° C. Triplicate samples were added and incubated two hours at RT. Plates were washed with Tris/NaCl/NP-40 (TNN) using a Skanwasher 300 (Skatron Instruments, Sterling, Va.). After washing, plates were incubated with biotin-coupled anti-IL-2 or anti-IFNγ (100 ng/well), for 1 hour at RT. Plates were washed and incubated an additional hour with Avidin-HRPO. Enzyme substrate (2,2′-azino-di[3-ethyl]-benzthiazoline sulfonate, Kirkegaard & Perry Laboratories, Inc., Gaithersburg, Md.) was added and the reaction was allowed to develop for 5 minutes. OD₄₀₅ was read on a Vmax, automated ELISA Plate reader (Molecular Devices, San Diego, Calif.). OD₄₀₅ values for CM were compared to appropriate cytokine standard. Data are reported as pg/ml of cytokine.

Flow Cytometry Staining for Anti-CD28 scFv and Treg Cells

To detect peripheral lymphocytes bearing anti-CD28 scFv, samples of peripheral blood, splenocytes or lymph nodes were harvested from mice injected ip with single chain antibody, or control Fab (50 μg), two hours previously. Cells (1×10⁷/ml) were blocked with anti-CD16/32, then stained with anti-CD3-A647, CD19-FITC (all purchased from PharMingen, San Diego, Calif.), and anti-6HIS-PE (R&D Systems, Minneapolis, Minn.). Stained samples were then washed with PBS-0.5% BSA and analyzed by flow cytometry. Dead cells were excluded using Hoechst 33258 (1 μg/ml). For peripheral blood samples, 100 μl of blood was blocked and stained with CD3, CD 19, and anti-6HIS, then fixed using BD FACSLyse reagent (BD Biosciences, San Diego, Calif.). Samples were washed with PBS-0.5% BSA, then analyzed by flow cytometry. To detect Treg cells, spleens were harvested and stained with anti-CD4-FITC and anti-CD25-PE (PharMingen, San Diego, Calif.)

Glucose Tolerance Test

Mice were tested for blood glucose levels on ad libitum food. Animals exhibiting elevated blood glucose (>200 mg/dl), were isolated and denied access to rodent chow overnight. Water was available ad libitum. Fasting blood glucose levels were obtained using an Elite XL glucometer (Bayer Corporation, Elkhart, Ind.). Mice were injected i.p., with 400 mg D-Glucose, dissolved in water. Blood glucose measurements were followed every fifteen minutes for a total of 90 minutes after glucose injection.

Reversal of Recent Onset Diabetes

Animals from the colony were routinely tested for urine glucose weekly. UgK⁺ animals, found by weekly screenings, were excluded from reversal studies. Remaining animals were reexamined two to three days later. Animals, which became UgK⁺ over that period, were then fasted overnight for initial glucose tolerance tests, the following day. After GTT, mice were then injected with either anti-CD28 scFv or control Fab (5-50 μg, ip injection), daily for 7-8 days. Glucose Tolerance Tests were performed on days 2, 4 and 7 of treatment. Area Under the Curve calculations were done on GTT results from individual mice and compared for assay of diabetes reversal.

Adoptive Transfer of Diabetes

Diabetic NOD mice were treated with 50 μg control Fab (H28.710) or anti-CD28 scFv ip daily for 7 days. 1×10⁷ spleen cells, isolated from treated mice, were injected ip into NOD.scid mice. Mice were monitored twice weekly for glucosuria.

Results

Intact Anti-CD28 Antibody can both Prevent and Exacerbate Diabetes Onset

Arreaza et al. previously demonstrated that use of an intact anti-CD28 antibody in weanling NOD females can prevent diabetes onset, by an IL-4 dependent mechanism ((1997) J. Clin. Invest. 100:2243-2253). As described herein, an injection of anti-CD28 antibody PV1 into weanling mice every other day from age 2-4 weeks, with an additional single dose at age 5, 6 and 7 weeks, prevented disease onset in a majority of mice. Previous studies, using hCTLA4-Ig had demonstrated no effect on diabetes onset, when used in adult mice (Lenschow et al. (1995) J. Exp. Med. 181:1145-1155). However, when intact anti-CD28 was injected into 8 week-old NOD females, an acceleration of diabetes onset was observed (p<0.0002; mice treated with control Ig, n=10; mice treated with anti-CD28, n=10). Female NOD mice were injected with 50 μg intact anti-CD28 antibody beginning at 8 weeks of age. Mice received intraperitoneal injections every other day for 2 weeks. Weekly testing for glucosuria began at 10 weeks of age and mice were recorded as diabetic after two consecutive positive readings.

Moreover, intact anti-CD28 also accelerated diabetes onset in male mice. For this experiment, male mice were examined at 21 weeks of age and diabetic mice (approximately 30%) were excluded. One week later, non-diabetic mice were reexamined. Mice, which had become diabetic over the week (i.e., tested positive for urine glucose), were also excluded. The remaining non-diabetic mice were divided into two groups. One was injected with control immunoglobulin (n=8), the other with anti-CD28 (n=7) (both 50 pg i.p., every other day) for two weeks. Treated mice were followed for diabetes onset until 31 weeks of age. Mice were recorded as diabetic after two consecutive positive readings. All male mice injected with intact anti-CD28 became diabetic within a few weeks of the initiation of treatment.

Therefore, intact anti-CD28 antibody delivers a positive signal in vivo which appears to costimulate the existing autoimmune response. Furthermore, it would appear that in non-diabetic mice, particularly in aged NOD males, a pathogenic population of T cells exist, which can be stimulated, or expanded by a costimulatory signal.

Blockade of Costimulation Responses In Vitro

To examine the specific blockade of B7/CD28 interactions in the at-risk diabetic mouse, an anti-CD28 single chain Fv was constructed. Heavy and light chain V regions were cloned from a cDNA library, derived from the PV1.17.10 cell line. Heavy and light chain fragments were joined by a Gly-Ser linker. A His-6 tag was added, to aid in protein purification. The resulting protein, approximately 28 kilodaltons, was tested for the ability to block costimulation responses.

The anti-CD28 scFv protein was tested in vitro for the ability to block costimulation dependent proliferation and cytokine responses. Spleen cells from NOD mice were cultured in vitro with soluble anti-CD3. Specifically, 1×10⁵ spleen cells from female NOD mice were incubated in 96 well round bottom plates with 10 μg/ml anti-CD3ε (1452C11) for 72 hours at 37° C. After 48 hours of culture, 100 μl of culture supernatant was harvested and assayed for cytokine production as described above under Materials and Methods for Example 5. Fresh medium was added to culture wells and plates were returned to incubator for 24 additional hours. ³H-Thymidine (0.5mCi/well) was added for the final 6 hours of culture. Anti-CD28 scFv was added over a wide range of concentrations, in particular, anti-CD28 scFv was added in three fold serial dilution, beginning at 30 ng/ml and proliferation was measured at 72 hours. Proliferation was inhibited with an IC₅₀ of 97 pg/ml. Proliferation in such cultures can be inhibited by CTLA4-Ig fusion proteins or the combination of antibodies to B7-1 and B7-2.

IL-2 production was also inhibited by anti-CD28 scFv. IL-2 levels present in culture supernatant were measured at 48 hours. Cytokine inhibition was more sensitive to costimulation blockade, with an IC₅₀ of 10 pg/ml. Culture supernatants concentrations of IFNγ were similarly reduced.

Anti-CD28 scFv Prevents Initiation of a Diabetic Response in Weanling Mice

Injection of intact anti-CD28 antibody has been demonstrated to prevent diabetes onset in NOD mice, by an IL-4 dependent mechanism (Arreaza et al. (1997) J. Clin. Invest. 100:2243-2253). Reagents, which target B7 molecules, such as CTLA4-1g, can also prevent diabetes onset, when used in weanling mice (Lenschow et al. (1996) Immunity 5:285-293). Thus, activation through CD28, as well as, blockade of B7 ligands has been reported to prevent diabetes onset. As described herein, the anti-CD28 scFv was used to examine the effects of blocking only CD28, and not CTLA4, in weanling NOD mice. Female NOD mice were injected with 50 μg anti-CD28 scFv every other day, for fourteen days, beginning at 2 weeks of age. A single 50 pg injection was also administered at 5, 6, and 7 weeks of age. Mice were followed for diabetes onset until 25 weeks of age, at which point, surviving mice were sacrificed and tissue examined histologically. As shown in FIG. 3, mice treated with anti-CD28 scFv, demonstrated a statistically significant decrease in diabetes incidence. Mice that became diabetic, did so with a substantial delay in onset. Histologic examination of pancreas of nondiabetic mice, revealed little or no lymphocytic infiltrate.

Anti-CD28 scFv Prevents Diabetes Progression

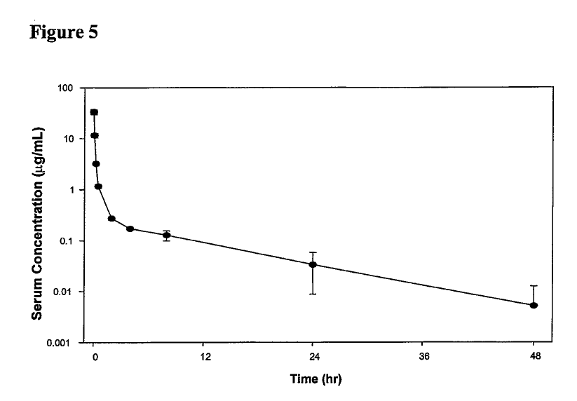

Specific CD28 blockade in adult mice was also examined. Previous studies with B7-directed costimulation blockade, failed to prevent diabetes onset in adult mice (Lenschow et al. (1995) J. Exp. Med. 181:1145-1155). Daily injection of single chain antibody for 14 days, beginning at 8 weeks of age, provided long-term protection from diabetes onset in 60% of the mice (FIG. 3B). Those mice that developed diabetes, did so in a delayed fashion. Daily injection of the anti-CD28 scFv was required, as alternate day injection from 8-10 weeks of age, did not delay diabetes onset. This is probably due to the relatively short in vivo half-life (<10 hours) of the single chain antibody (FIG. 5). In a pharmacokinetic evaluation of anti-CD28 scFV in vivo, BALB/c mice were treated with 20 mM KI in drinking water for 3 days prior to study initiation. At dosing, mice were then injected with a mixture of 125I labeled and unlabeled anti-CD28 scFV, at a total dose of 1 mg/kg. Three animals were bled by cardiac puncture at 5 minutes, 15 minutes, 1, 3, 6, 24, 28, and 72 hours and blood samples were assayed for radioactivity.

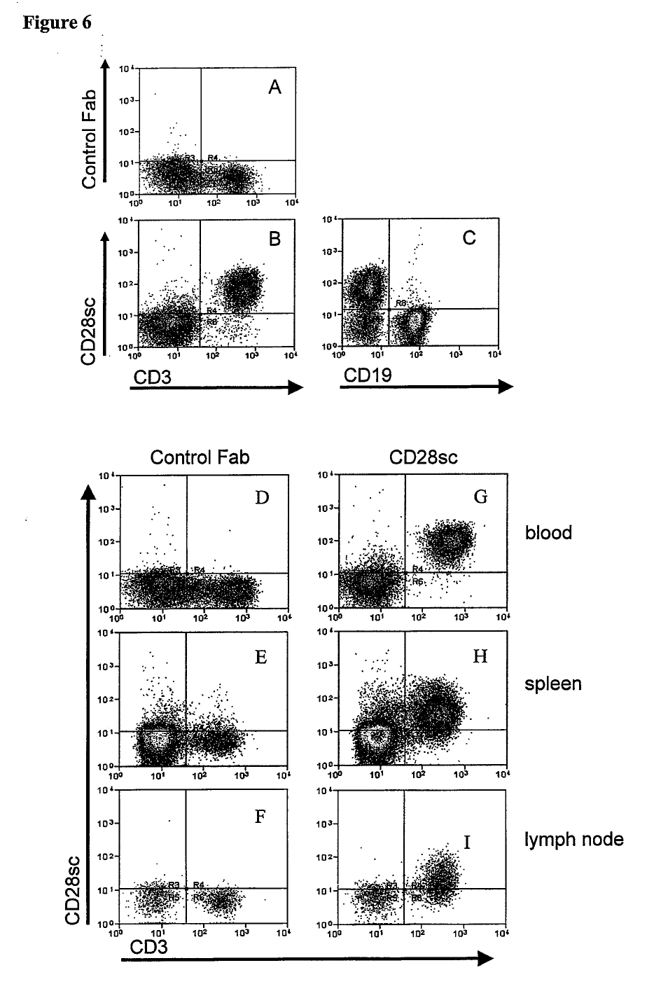

Data in FIG. 6 demonstrate the rapid and complete coverage of T cell CD28 upon injection of anti-CD28 scFv. Flow cytometric examination of peripheral blood T cells 2 hours after ip injection of single chain antibody revealed >98% of circulating T cells staining with single chain antibody (FIG. 6B). Peripheral blood B cells did not stain with anti-CD28 scFv (FIG. 6C). Examination of secondary lymphoid tissues demonstrated staining of splenic and lymph node T cells within 2 hours of single chain antibody injection (FIGS. 6H-I).

Islet Inflammation but not Infiltration in Anti-CD28 scFv Treated Mice

Nondiabetic mice, which had been treated with control Fab or anti-CD28 scFv were sacrificed at 30 weeks. Histological examination of the mice treated with single chain antibody daily from 8 to 10 weeks of age, revealed a distinct phenotype. In any individual surviving mouse, there were some islets with no lymphocytic infiltrate and some islets, which had been destroyed by invading lymphocytes. However, all nondiabetic mice shared a common phenotype for a large number (>50%) of islets examined. Massive accumulation of lymphocytes was present outside the islet in these mice.

It would appear that interruption of CD28 signaling, even comparatively late in diabetogenesis, can affect diabetes onset by preventing islet infiltration. Compiled histology data (Table 1) shows data from these mice as well as animals treated with anti-CD28 scFv as weanlings. As might be expected from blockade of the autoimmune response at an early stage, nondiabetic mice treated from age 2 weeks showed little islet infiltration. The relatively low level of infiltration from the few control mice, in either group, which had not become diabetic by 30 weeks of age is to be expected.

TABLE 1
Compiled histology data of animals treated
with control antibody and anti-CD28 scFv

| | Timing | # mice | # islets | Percentage of islets scoring | | | | |
|---|---|---|---|---|---|---|---|---|
| Treatment | (age) | tested | tested | 0 | 1 | 2 | 3 | 4 |
| Control Fab | 2-5 weeks | 3 | 103 | 16.5 | 54.4 | .8 | 4.9 | 16.5 |
| Anti-CD28 scFv | 2-5 weeks | 7 | 133 | 67.8 | 13.3 | 2.8 | 4.9 | 4.2 |
| Control Fab | 8-10 weeks | 3 | 101 | 25.7 | 45.5 | 12.9 | 7.8 | 7.9 |
| Anti-CD28 scFv | 8-10 weeks | 6 | 240 | 25.4 | 52.9 | 7.9 | 7.7 | 5.8 |

Anti-CD28 scFv does not Induce Treg Cells

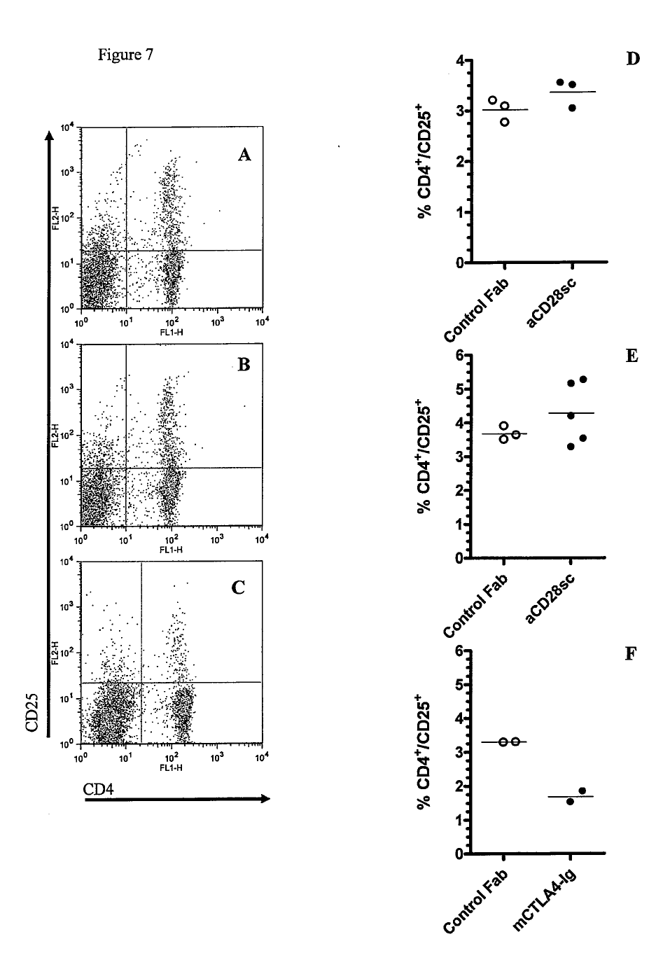

Regulatory T cells have been demonstrated to impact diabetes onset in the NOD model of type I diabetes (Akhtar et al. (1995) J. Exp. Med. 182:87-87; Sai et al. (1996) Clin. Exp. Immuol. 105:330-337; Cameron et al. (1997) J. Immunol. 159:4686-4692). Previous reports have observed that blockade of B7 by murine CTLA4-Ig can reduce CD4+/CD25⁺ Treg levels in NOD (Salomon et al. (2000) Immunity 12:431-440). To determine whether Treg populations were affected by treatment with specific CD28 blockade, NOD mice were treated with single chain antibody beginning at either 2 weeks or 8 weeks of age. Mice treated with anti-CD28 scFv from 2-5 weeks of age with additional injections at 7 and 8 weeks did not develop diabetes (FIG. 3A) or islet infiltration. Spleen cells from mice treated with the same therapeutic regimen showed similar percentages of CD4⁺/CD25⁺ Treg as control mice (FIG. 7B, D). In addition, NOD mice injected with anti-CD28 scFv from 8-10 weeks of age showed reduced and delayed diabetes onset (FIG. 3B) with inflammation but not infiltration of pancreatic islets. Examination of spleen cells from NOD mice treated from 8-10 weeks of age with anti-CD28 scFv also demonstrated no significant increase in Tregs (FIG. 7E). Treatment of NOD mice with mCTLA4-Ig from 8-10 weeks of age accelerates diabetes onset. Spleen cells from mCTLA4-Ig-treated mice have reduced levels of Treg cells (Salomon et al. (2000) Immunity 12:431-440).

Anti-CD28 scFv can Delay Loss of Glucose Tolerance

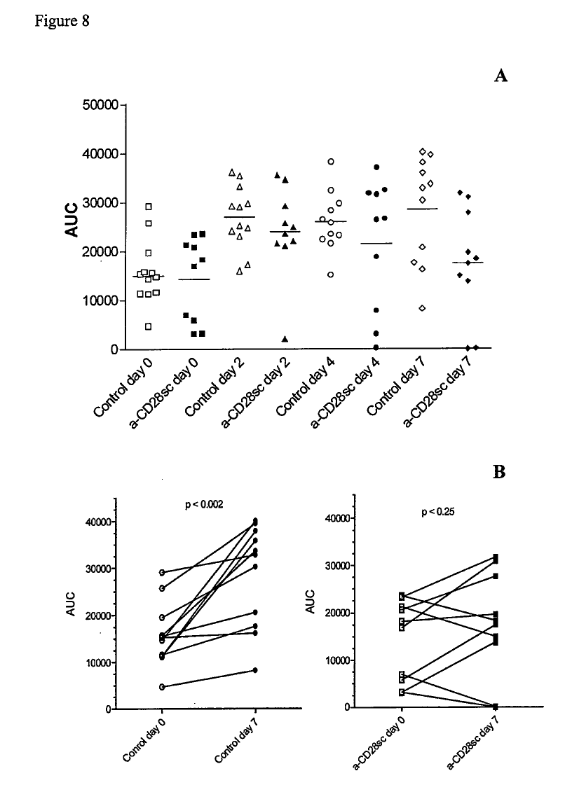

Recent disease onset in the NOD mouse was also examined by performing Glucose Tolerance Tests (GTT) as a measure of functional insulin production, in mice that had been diabetic for less than four days. Recent onset diabetics were then treated aggressively with anti-CD28 scFv or control Fab. Follow up GTT were performed on treated and control animals on days 2, 4 and 7 of treatment. Individual GTT results are disclosed in FIG. 8. Data are represented as AUC measurements for the 90-minute duration of the Glucose Tolerance Test. As shown in FIG. 8A, mice treated with control Fab showed a steady loss of glucose tolerance over the course of seven days. Mice treated with anti-CD28 scFv showed an increased AUC on days 2 and 4 but median AUC scores on days 0 and 7 seven were not statistically distinguishable (FIG. 8B). Some mice in the anti-CD28 scFv-treated group transiently returned to normal glucose tolerance. The anti-CD28 scFv group tested on day 0 shows two separable populations of mice, with higher and lower AUC measurements. To ensure that the lower points on the anti-CD28 scFv day 7 data were not solely derived from those mice with lower initial GTT results, individual mice were tracked over the course of the experiment. Six mice out of the group of ten show a steady decrease in the ability to respond to exogenous glucose, as evidenced by a positive slope of the line connecting the day 0 and day 7 AUC values. However, four of the ten treated mice had substantially lower AUC values indicating an increasing ability to respond to glucose challenge. Two of the four responding mice represent two of the three highest AUC measurements recorded in their treatment group on day 0. All of the mice being treated with Control Fab fragments showed increasingly poor GTT results over the course of treatment.

Anti-CD28 scFv Reduces Autoimmune Reactivity

Chatenoud et al. have demonstrated reversal of diabetes onset in NOD mice by treatment with anti-CD3 ((1994) Proc. Natl. Acad. Sci. 91:123-127). Diabetes reversal was not demonstrated in anti-CD28 scFv treated mice. However, specific CD28 blockade did impact the ability of spleen cells form recent onset diabetic mice to transfer diabetes. Recent onset diabetic NOD mice were treated with either control Fab or anti-CD28 scFv (50 μg daily intraperitoneal injections) for one week after diabetes onset. Spleen cells (1×10⁷) from treated mice were harvested and injected into NOD-scid recipients. Mice injected with spleen cells from mice treated with single-chain anti-CD28 demonstrated a marked delay in diabetes onset as compared to mice which received cells from control Fab injected mice (p<0.0001; control Fab, n=5; anti-CD28 scFv, n=12). Mice were recorded as diabetic with a second positive urine glucose test within 24 hours of the first positive test.

Discussion

The role of T cells in the initiation, as well as the pathogenesis of Immune Mediated Diabetes, in man, and in the NOD mouse is unquestioned (Castano, L., and G. S. Eisenbarth (1990) Ann. Rev. Immunol. 6:647-679; Haskins, K., and D. Wegmann. (1996) Diabetes 45:1299-1305; Shoda et al. (2005) Immunity 23:115-126). Similarly, it is well established that CD28 provides a costimulatory signal necessary for optimal activation of T cells (Lenschow et al. (1996) Annu. Rv. Immunol. 14:233-258; Green et al. (1994) Immunity 1:501-508; Chambers, C. A., and J. P. Allison (1997) Curr. Opin. Immunol. 9:396-404). Thus, it came as no surprise, that early studies into the role of B7-mediated costimulation, revealed a central role for accessory molecules in the development of the murine diabetogenic response. Intervention in the ‘normal’ disease process with reagents, which putatively targeted both B7.1 and B7.2, such as hCTLA4-Ig, could prevent disease onset, if the therapeutic was administered before significant development of the autoimmune response had occurred (Lenschow et al. (1996) Immunity 5:285-293). Similar efficacy was observed, using CTLA4-Ig in Experimental Autoimmune Encephalomyelitis, Collagen-Induced Arthritis, murine models of Systemic Lupus Erythematosus and multiple murine allograft rejection model (Chang et al. (1999) J. Exp. Med. 190:733-740; Daikh, D. I., and D. Wofsy (2001) J Immunol 166:2913-2916; Finck et al. (1994) Science 265:1225-1227; Karandikar et al. (1998) J. Neuroimmunol. 14:10-18; Larsen et al. (1996) Nature 381:434-438; Lin et al. (1993) J. Exp. Med. 178:1801-1806; Newell et al. (1999) J Immunol 163:2358-2362; Pearson et al. (1994) Transplantation 57:1701-1706; Sayegh et al. (1997) Transplantation 64:1646-1650; Webb et al. (1996) Eur. J. Immunol. 26:2320-2328; Zheng et al. (1999) J Immunol 162:4983-4990).

Further dissection of the B7/CD28/CTLA4 pathway complicated the analysis of costimulation-dependent diabetogenesis. Given the redundant binding of CD28, by both B7-1 and B7-2, it could have been expected, that in vivo blockade of B7 by monoclonal antibodies specific for either molecule alone would not produce protection from autoimmune disease. However, protection from diabetes onset was precisely what was observed when NOD mice were injected with antibodies, specific for B7-2. Protection occurred, despite the fact that interaction between B7-1 and CD28 would not have been interrupted by anti-B7-2. Specific blockade of B7-1/CD28/CTLA4 interaction was addressed by using monoclonal anti-B7-1, with exactly the opposite result. Antibodies to B7-1, as well as the combination of anti-B7-1 plus anti-B7-2, exacerbated disease onset (Lenschow et al. (1995) J. Exp. Med. 181:1145-1155). B7 knockout mice, bred onto the NOD background, confirmed these results (Salomon et al. (2000) Immunity 12:431-440). The discrepancy between prevention of diabetes onset by CTLA4-Ig and exacerbation of disease by the combination of antibodies, can be explained by differential affinity for B7s. The human CTLA4-Ig fusion protein, used by Lenschow et al. (Lenschow et al. (1995) J. Exp. Med. 181:1145-1155) does not inhibit B7-1 mediated costimulation with equal efficiency to its inhibition of B7.2 (Collins et al. (2002) Immunity 17:201-210). Use of this reagent in vivo, mimics the use of anti-B7-2 antibody alone. This is confirmed by the more efficient blockade of both B7-1 and B7-2 costimulation, by murine CTLA4-Ig, as well as, the fact that mCTLA4-Ig exacerbates NOD diabetes (Salomon et al. (2000) Immunity 12:431-440; Collins et al. (2002) Immunity 17:201-210).

CD28-mediated costimulation in diabetogenesis has been examined by other laboratories. It had been previously reported that NOD T cells were hyporesponsive to TCR signaling, rendering them functionally anergic, possibly leading to initiation of diabetes (Zipris et al. (1991) J. Immunol. 146:3763-3771). Arreaza et al. reported that anti-CD28 could augment T cell responsiveness in vitro, leading to more robust proliferation and the production of IL-4. Furthermore, these investigators injected intact anti-CD28 antibody into weanling NOD females to protect against diabetes onset (Arreaza et al. (1997) J. Clin. Invest. 100:2243-2253). The anti-CD28 (clone 37.51) used in these experiments, was distinct from the PV1.17.10 clone used herein. These investigators found that injection of anti-CD28, beginning at 2 weeks of age, promoted an IL-4-dependent mechanism, which protected against insulitis and diabetes onset. Delay of treatment, until 5 weeks of age, did not protect against diabetes onset. The authors hypothesized that CD28 signaling was a requisite component of Th2 development in vivo and that a lack of Th2 cell activity was responsible for IMD development in the NOD mouse. In support of this argument is the work done by Salomon et al., who crossed the CD28 knockout mouse onto the NOD background (Salomon et al. (2000) Immunity 12:431-440). Previous studies with CD28 KO mice, on non-autoimmune backgrounds, had demonstrated poor in vivo immune responses, including, delayed-type hypersensitivity, antibody isotype switching and weak, but not absent graft rejection (Sharpe, A. H. (1995) Curr. Opin. Immunol. 7:389-395). Given the weak immune responses of CD28KO mice on conventional backgrounds, one might have predicted little or no diabetes onset in when the CD28KO mice were crossed onto the NOD background. To the contrary, CD28KO NOD mice showed an aggressive disease onset and near complete disease penetrance (Lenschow et al. (1995) J. Exp. Med. 181:1145-1155; Salomon et al. (2000) Immunity 12:431-440). Taken together, these data indicated that an early Th2 autoreactive phenotype protected NOD mice from diabetes onset. Intact anti-CD28 antibody, promoted this protective response. NOD mice with a deleted CD28 gene were unable to mount the protective Th2 response and thus showed an acceleration of diabetes onset. Non-autoimmune prone mice have demonstrated the ability to reject allografts in the absence of a functional CD28 gene (Kawai et al. (1996) Transplantation 61:352-355; Pearson et al. (1997) Transplantation 63: 1463-1469).

As described herein, some of these studies were repeated using a different anti-CD28 antibody (PV1). Injection of intact PV1 into NOD females, beginning at 2 weeks of age, prevented diabetes onset. By eight weeks of age, anti-CD28 acts as an accelerant and promotes the development of diabetes in the at-risk animal. Intact anti-CD28 also promoted diabetes onset when used in male mice. It would appear from these data, that use of intact anti-CD28 in vivo, can costimulate the ongoing autoimmune response. In the weanling NOD, that response is a Th2 phenotype and if promoted can protect from disease onset. In the adult female, the autoimmune response is more Th1 in nature. Costimulation of this Th1 response accelerates the onset of diabetes. These data also indicate that diabetes onset can be accelerated in adult mice by exogenous costimulation. This may be analogous to those of Andre-Schmutz et al., who reported that diabetes onset could be ‘synchronized’ in at-risk animal populations by treatment with cyclophosphamide (Harada, M., and S. Makino (1984) Diabetologia 27:604-606; Yasunami, R., and J. F. Bach (1988) Eur. J. Immunol. 18:481-484).

Both PV1 and the 37.51 antibodies can act as a positive signal (Mandelbrot et al. (1999) J. Exp. Med. 189:435-440; Szot et al. (2000) Transplantation 69:904-910). Inasmuch as, these antibodies can signal in vivo, it is difficult to reconcile use of the antibodies with the B7 targeted reagents used to block B7/CD28/CTLA4 interactions and thus prevent diabetes onset in the NOD mouse. To address specific blockade of B7/CD28 interaction in isolation, we constructed the anti-CD28 single chain Fv used in these experiments. The scFv retains potent binding activity for CD28 with comparable binding kinetics in BIACORE experiments to intact PV1 (L. Fitz, unpublished). The anti-CD28 scFv is a monomer, incapable of delivering a costimulatory signal in vitro, unless bound to an insoluble matrix. As such, its only activity in vivo would be as a blocking reagent. Use of this CD28-specific blocking reagent has proven effective in preventing diabetes onset in NOD mice at two separate stages of disease development. Following the same protocol used for intact anti-CD28, CTLA4-Ig and anti-B7-2, we injected 50 μg of PV1 scFv every other day for 14 days, beginning at 2 weeks of age. Additional single injections took place at ages 5, 6 and 7 weeks. Mice treated with this reagent were protected from diabetes onset in the majority of cases. Histologic examination of the nondiabetic treated mice, revealed insignificant islet infiltrates, consistent with an effective blockade of the initiation of the autoimmune response in the two to three week old mouse.

In contrast to data reported with anti-B7-2, or hCTLA4-Ig, treatment of adult mice with anti-CD28 scFv, also prevented diabetes onset. Mice injected with PV1 scFv every day were protected from diabetes onset for up to 20 weeks after cessation of treatment. Daily treatment was necessary, as treatment on alternate days was insufficient to prevent diabetes. This is likely due to the relatively short half-life, approximately 10 hours in vivo, of the anti-CD28 scFv (FIG. 5).

Adult mice treated with anti-CD28 scFv, showed a histologic phenotype, distinct from that observed by treating weanling mice. Nondiabetic 30 week-old mice, which had been treated with anti-CD28 scFv from age 8 to 10 weeks, demonstrated a massive peri-islet inflammation, without infiltration, in more than 50% of the islets examined. Although protected from diabetes onset, these mice did not present with histologically normal pancreatic islets. Lymphocytes have trafficked to the islets, but failed to infiltrate the islet itself. This phenotype may illustrate the need for a costimulation-dependent event at the site of islet infiltration. The phenotype of peri-islet accumulation of lymphocytes, without frank insulitis is quite similar to that observed in the diabetes resistant NOR strain, which also shows marked per-islet inflammation. NOR mice and NOD mice are disparate at the Idd5 locus, originally thought to encode CD28, CTLA4 and ICOS genes (Prochazka et al. (1992) Diabetes 41:98-106; Serreze et al. (1994) J. Exp. Med. 180:1553-1558). Further refinement of the mapping of Idd5 has removed CD28 as a candidate gene for Idd5 (Wicker et al. (2004) J Immunol 173:164-173).

A similar need for restimulation at the site of the target organ was reported by Chang and coworkers (Chang et al. (1999) J. Exp. Med. 190:733-740). Adoptive transfer of encephalitogenic cells from wild-type mice failed to induce EAE in B7-1/B7-2 double knockout mice. The lack of B7-dependent restimulation of antigen-primed cells in the target organ prevented disease onset.

By eight weeks of age, the majority of female NOD mice have an ongoing autoimmune response. Islet-reactive T cells in the spleen of NOD mice are changing from a protective Th2 to a pathogenic Th1 phenotype (Kaufman et al. (1993) Nature 366:69-71; Tisch et al. (1993) Nature 366:72-75). Cytokine transcripts are readily detectable in isolated 8 week-old mice (Faulkner-Jones et al. (1996) Autoimmunity 23:99-110; Rothe et al. (1997) Journal of Autoimmunity 10:251-256). During this ongoing autoreactive response, disruption of the costimulatory signals delivered through CD28 can still have a profound effect on diabetes development. Whether this occurs in the peri-islet space or in the draining lymph node is not known, but clearly, a costimulation-dependent requirement exists for the transition from inflammatory to infiltrating insulitis.

Further evidence that costimulation through CD28 continues throughout the disease process comes from experiments in recent onset diabetics (FIG. 8). NOD females, which had tested positive for glucosuria, accompanied by elevated blood glucose, were treated with anti-CD28 scFv. Chatenoud et al. ((1994) PNAS 91:123-12715) have reported that treatment of diabetic NOD mice with intact anti-CD3, near the time of disease onset, permanently reverses diabetes in the majority of animals. We have been unable to duplicate these results, by using CD28-specific blockade. However, short-term improvements in GTT results were obtained in some mice. This data, combined with the impaired ability of spleen cells from anti-CD28 scFv treated mice to adoptively transfer disease, clearly demonstrates an ongoing pathogenic response, dependent on CD28 signaling, present at all points of the disease process.

The mechanism of protection in anti-CD28 scFv mice does not appear to be due to increases in CD4⁺/CD25⁺ regulatory T cell populations. FIG. 7 does not indicate an increase in the number of Treg cells in mice treated beginning at either 2 or 8 weeks of age. It is possible that Treg cells in these mice are more active, as a potential interaction between B7 and CTLA4 molecules was not prevented. Signaling through CTLA4 has been proposet to enhance Treg cell activity (Bachmann et al. (1999) Journal Of Immunology (Baltimore, Md.: 1950) 163:1128-1131; Read et al. (2000) The Journal Of Experimental Medicine 192:295-302; Takahashi et al. (2000) The Journal Of Experimental Medicine 192:303-310). The earlier work by Arreaza et al. (Arreaza et al. (1997) J. Clin. Invest. 100:2243-2253), using an agonistic anti-CD28 antibody, demonstrated an IL-4 mediated regulatory process of protection from diabetes onset. The results presented herein are more indicative of a blockade of effector T cell induction and function, inasmuch as, the single chain antibody will only block CD28 interaction with B7 ligands.

From these data and those published earlier, it is clear that CD28 signaling is critical in both the development of and the protection from Immune Mediated Diabetes onset. Active signaling through CD28 can promote long lasting protection from diabetes onset, when such therapeutic intervention is done sufficiently early in disease development. However, it is equally clear that the very same active signaling through CD28 at a later stage in disease development can hasten the onset of disease. Design of a reagent, which can specifically block CD28 interactions with its ligands, appears to enable intervention, in the diabetogenic process, both at the initiation of autoimmunity, as well as, later in disease development. Furthermore, short-term intervention, near the time of diabetes onset, can have long-lasting effects. These data make the specific targeting of CD28 an attractive concept for therapeutic intervention in IMD.

### Example 6

### Selective CD28 Blockade Attenuates Acute and Chronic Cardiac Allograft Injury

Immunocyte responses mediated by the CD28 family of costimulatory molecules determine the balance between regulatory and pathogenic effector mechanisms after initial antigen exposure. Targeting the CD28/B7 pathway by use of CTLA4-Ig reagents (Belatacept, Abatacept) which directly bind B7s is a promising alternative to prevent autoimmunity (Alegre, M. L., Frauwirth, K. A., & Thompson, C. B., Nat. Rev. Immunol. 1, 220-228 (2001); Kremer, J. M. et al., N. Engl. J. Med. 349, 1907-1915 (2003)) and part of a calcinerin-free maintenance immunosuppressive regimen in renal transplantation (Larsen et al., (1996) Nature 381:343-438; Larsen et al. (2005) Am. J. Transplant. 5(suppl. 11): 293(abstract); Larsen et al. (2005) Am. J. Transplant 5:443-453; Vincenti et al. (2005) N. Engl. J. Med. 353:770-781; Larsen et al. (2006) Am. J. Transplant. 6:876-883). The current paradigm holds that constitutively expressed CD28 binds B7 to provide a stimulatory signal important for sustaining T cell proliferation and augmenting proinflammatory responses. CTLA-4, another B7 ligand induced on T-cells subsequent to high affinity TCR ligation, delivers antiproliferative (Walunas, T. L. et al. (1994) Immunity 1, 405-413; Tivol, E. A. et al. (1995) Immunity 3, 541-547); Waterhouse, P. et al. (1995) Science 270, 985-988) and/or tolerogenic signals to T-cells, and to B7-bearing antigen presenting cells (APCs), in which it triggers increased indoleamine dioxygenase (IDO) (Mellor, A. L. et al. (2004) Int. Immunol. 16, 1391-1401).

However, several recent observations show that B7-directed blocking strategies deprive the evolving immune response of CTLA-4-driven signals crucial to development of antigen-specific peripheral regulatory T-cells. Blocking the CD28/B7 pathway by ligation of B7, using either a CTLA-4 analogue (Adams (2002) Diabetes 51:265-270) or antibodies against B7 family members (Kirk et al. (2001) Transplantation 72:337-384; Haanstra (2003) Transplantation 75:637-643), does not reproducibly induce tolerance across a full MHC mismatch in rodents or primates. CTLA-4 signaling is required for the induction of peripheral T cell tolerance to soluble antigens (Akiyama et al. (2002) Transplantation 74:732-738; Greenwald et al. (2001) Immunity 14:145-155; Issazadeh et al. (1999) J. Immunol. 162:761-765; Perez et al. (1997) Immunity 6:411-417), tumors (Shrikant et al. (1999) Immunity 11:483-493) and allografts (Markees et al. (1998) J. Clin. Invest. 101:2446-2455; Zheng et al. (1999) J. Immunol. 162:4983-4990; Tsai et al. (2004) Transplantation 77:48-55). Further, selective agonistic ligation of CTLA-4 attenuates in vivo T cell responses and prevents development of autoimmunity (Fife et al. (2006) J. Clin Invest. 116:2252-2261; Ansari and Sayegh (2006) J. Clin Invest. 116:2080-2083).

Based on these considerations, selective inhibition of CD28 should prevent maturation of pathogenic effectors, while promoting preferential CTLA4-driven expansion of antigen-specific regulatory T-cells (T regs) as well as emergence of regulatory APCs. Described herein is the use of non cross-linking anti-CD28 receptor antagonists in murine and primate heart transplant models.

Material and Methods

Reagents: Non-activating mouse CD28-specific scFv antibody fragment (αm28 scFv) was developed from the well-characterized hamster antibody clone PV1.17.10 as described above. Similarly, a non-activating human CD28-specific scFv antibody fragment was developed from the well-characterized clone CD28.3, and linked to alpha-I anti-trypsin (αh28scAT) to prolong its serum half-life (Vanhove, B. et al. (2003) Blood 102, 564-570). αh28scAT was purified from transformed CHO cells supernatant by ion exchange chromatography (Mustang Q, Pall Biosepra, Paris, F). αh28scAT cross-reacts with CD28 from cynomolgus monkey and baboon, but not from rat and mouse. Anti-mouse CD 154 antibody (MR1) was purchased from Bioexpress (West Lebanon, N.H.). Anti-human CD 154 (IDEC-131) was a kind gift from Biogen-IDEC (San Diego, Calif.). hCTLA-Fc was purchased from Chimerigen LLC (Allston, Mass.). Anti-human CTLA4 (clone BNI3) was purchased from BD Biosciences Pharmingen (San Diego, Calif.). Purified hamster IgG (Jackson ImmunoResearch Laboratories, West Grove, Pa.) and human IgG1 (Sigma, St Louis, Mich.) were used as controls.

Animals: Six to 10-week-old C57BL/6 (H-2^{b}), BALB/c (H-2^{d}), and C3H/HeJ (H-2^{k}) male mice were obtained from The Jackson Laboratory (Bar Harbor, Me.). Cynomolgus monkeys (Macaca fascicularis) (2 to 3 kg) were obtained from Covance Research Products (Alice, Tex.) and Three Springs Scientific Inc (Perkasie, Pa.). Simian-type blood grouping in saliva was by Primate Blood Group Laboratory (Tuxedo, N.Y.). Female recipients were paired with blood type compatible, mixed lymphocyte reaction (MLR)-mismatched (SI>3 in MLR) male donors (actual SI range: 5-20). Animals were housed under conventional conditions and used according to the guidelines of the Institutional Animal Care and Use Committee (IACUC) of the University of Maryland Medical School. Protocols approved by the IACUC were carried out in compliance with the Guide for the Care and Use of Laboratory Animals (HHS, NIH Publication 86-23, 1985).

Cell isolation and proliferation assays: For mouse mixed lymphocyte reaction (MLR) experiments, splenocytes from naive BALB/c and C57BL/6 mice were used as responder and stimulator cells respectively. Mouse responder cells were cocultured with irradiated stimulator cells (3×10⁵ each/well) in RPMI containing 10% FBS, gentamycin and 2-,mercaptoethanol in 96 round bottom plates.

For cynomolgus MLR, blood was collected from naive animals and peripheral blood mononuclear cells (PBMC) isolated as described (Pierson, R. N., III et al. (1999) Transplantation. 68, 1800-1805). Responder cells were cocultured in 96 round bottom plates with irradiated stimulator cells (10⁵ each/well) in RPMI supplemented with 10% human AB serum (Atlanta Biologicals, Lawrenceville, Ga.) and gentamycin (Gibco (Invitrogen Corp.), Carlsbad, Calif.). For human MLR, human PBMC were stimulated with allogeneic irradiated PBMC and cultured in the presence of the indicated amount of αh28scAT, with or without 10 pg/ml of the anti-CTLA-4 BNI3 Mab. Antibodies (anti-CD28, anti-CD154, anti-CTLA-4, or irrelevant IgG) were added at indicated concentrations. After 5 days, proliferation of responding T cells was assessed by measurement of 3H-thymidine incorporation.

MLR results were expressed as the stimulation index (SI) relative to autologous control or as the measured 3H-thymidine incorporation (CPM) after subtraction of specific CPM for the responding and stimulating cells alone. Purified hamster IgG and human IgG1 were used as specificity controls for murine and primate reactions, respectively.

Cardiac transplantation and treatment protocols in mice: Vascularized heterotopic hearts from C57BL/6 and BALB/c donors were transplanted into the abdomen of BALB/c recipients using the microsurgical technique of Corry et al. (Corry et al. (1973) Transplantation 16, 343-350). Graft survival was monitored by daily palpation. Rejection was defined as complete cessation of the palpable heartbeat and was confirmed histologically. In initial dosing experiments, recipients were treated with αm28scFv at 200 μg on days 0-2, 2, 2-4, or 0-13; or 50 μg αm28scFv on days 0-13. Twice daily treatment demonstrated optimal efficacy, presumably due to the relatively short half-life (10 hours) of αm28scFv. All recipients described in this manuscript received αm28scFv 200 μg IP on days 0-13 (n=12), MR1 (Sho et al. (2003) Transplantation 75, 1142-1146; Sho, M. et al. (2002) Ann. Surg. 236, 667-675) (250 μg IP on day 0, n=20), CsA (Sho et al. (2003) Transplantation 75, 1142-1146; Sho, M. et al. (2002) Ann. Surg. 236, 667-675) (400 μg IP on days 0-3, n=9), αm28scFv plus MR1 in combination (n=17), or αm28scFv plus CsA in combination (n=18). Additional control allograft (n=16) and isograft (n=5) recipients were left untreated.

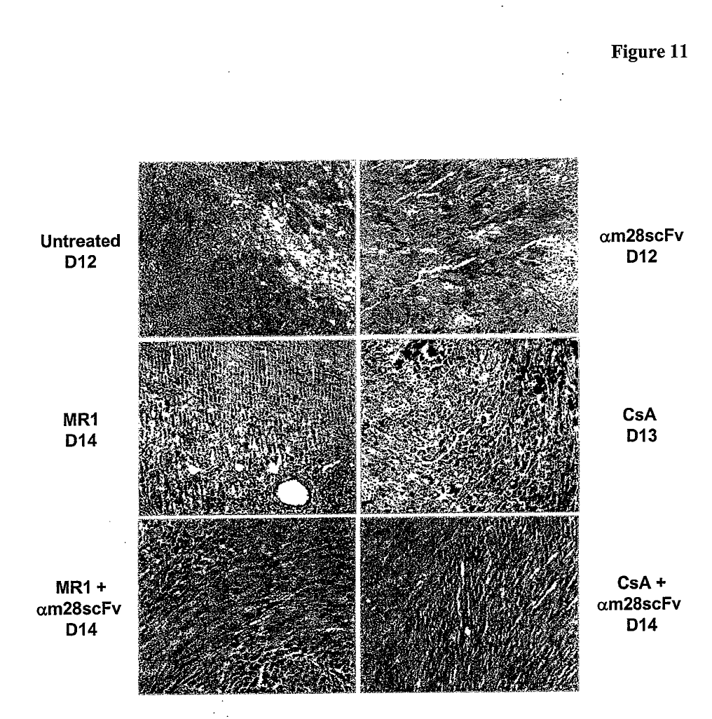

Mouse graft histology: At the time of explant, cardiac grafts were trisected. The apex was immediately snap-frozen for molecular analysis. The basal part of the heart was fixed in 10% buffered formalin, embedded in paraffin, sectioned, and stained with H&E and Verhoeff s elastin according to standard procedures. The middle part was frozen in OCT compound for immunohistochemistry. Elastin-stained sections were used to assess transplant arteriosclerosis. The incidence (proportion of vessels affected) and grade (severity) of arteriosclerosis were scored, with severity graded as follows: 0 represents a normal artery; 1, 1%-20% occlusion; 2, 21-40% occlusion; 3, 41-60% occlusion; 4, 61-80% occlusion; and 5, >80% occlusion), as described (Sho et al. (2003) Transplantation 75, 1142-1146; Sho, M. et al. (2002) Ann. Surg. 236, 667-675). Grafts that failed within 60 days in animals treated with CsA, MR1, or anti-CD28 monotherapy exhibited Grade 4 acute cellular rejection (FIG. 11).

Skin transplantation in mice: Full thickness ear skin allografts (1 cm²) taken from (BALB/c) or third party (C3H/He) donors were transplanted on the dorsal thorax of recipient mice and secured using plastic adhesive bandages. The graft survival was followed by daily inspection. Rejection was defined as more than 80% graft necrosis.

Detection of antidonor alloantibody in mice. Donor-reactive antibodies were measured by flow cytometry as previously described (Sho et al. (2003) Transplantation 75, 1142-1146; Sho, M. et al. (2002) Ann. Surg. 236, 667-675). Briefly, splenocytes (0.5×10⁶) of native C57BL/6 donor strain animals were incubated for 30 min at 4° C. with 1:10, 1:100, 1:1000, 1:10000 dilutions of mouse sera obtained from native BALB/c, naive C57BL/6, or BALB/c recipients of a prior heart transplant. The cells were washed twice, stained with biotin-conjugated antibody against mouse IgGl or IgG2a (BD Biosciences) for 30 minutes at 4° C., followed by PE-conjugated streptavidin mixed with FITC-conjugated anti-mouse CD3 (clone 145-2C11). Flow cytometry analysis was carried out on a FACS calibur flow cytometer, and data were analyzed using CellQuest software (BD Immunocytometry Systems, San Jose, Calif.). Results were expressed as the percentage of positive cells among gated CD3+ T cells, after subtraction of the autologous control. In long-term recipients, positivity for IgG2a antibody was higher when serum was diluted 1:100 than 1:10, suggesting the presence of IgG2a competing with IgG1 at high (less physiologic) serum dilutions. Therefore, a serum dilution of 1:10 was considered in all subsequent analysis.

Isolation of cell populations: Single cell suspensions were prepared from the spleen and from the draining (lateral aortic) lymph node of murine allograft recipients by mincing with forceps and passage of the resulting cell suspension through nylon mesh of 100-μm pore size. In addition, in selected recipients at day 10-12, graft-infiltrating lymphocytes (GIL) were isolated by mincing the graft and incubating the resulting fragments for 30 min in medium containing 1 mg/ml collagenase type 4 (Worthington Biochemical, Freehold, N.J.), 1 mg/ml soybean trypsin inhibitor (Sigma-Aldrich, St. Louis, Mo.), and 0.1 mg/ml DNase (Roche, Indianapolis, Ind.) as previously described (Wang, D. et al. (2004) J. Immunol. 172, 214-221). Lymphocytes were isolated by Ficoll-gradient centrifugation.

FACS analyses: Cells were surface stained for 15 min at 4° C. with FITC-conjugated anti-CD4 mAb (GK1.5, BD Pharmingen, San Diego, Calif.), APC-conjugated anti-CD25 (PC61, BD Pharmingen) and Cychrome-conjugated anti-CD3 mAb in PBS supplemented with 1% BSA and 0.2% sodium azide. For Foxp3 staining, surface stained T cells were incubated in permeabilization buffer (eBioscience, San Diego, Calif.) for 16-18 h at 4° C. before performing intracellular staining with FITC-conjugated anti-Foxp3 (eBioscience, San Diego, Calif.). Lymphocyte populations were gated by forward/side scatter analysis to exclude debris. Data analysis and graphic display were conducted using CellQuest software.

Mouse ELISPOT assay: ELISPOT plates (Cellular Technology Ltd., Cleveland, Ohio) were coated overnight at 4° C. with anti-IFN-γ, anti-IL-2, anti-IL-4 (BD Biosciences Pharmingen), and anti-IL-10 (eBioscience) capture antibodies (5 μg/ml). The plates were then blocked with RPMI containing 10% FBS for 1 hour at 37° C. Responder cells (3×10⁵ [IFNg, IL-2, IL-4] or 5×10⁵ splenocytes per well [IL-10]) were cocultured with irradiated stimulator cells (1:1 ratio) and cultured for 24 hours (IFN-γ, IL-2) or 41 hours (IL-4, IL-10). After washing with deionized water and PBS/0.05% Tween 20, 2 μg/ml biotinylated anti-IFN-γ, —IL-2, —IL-4 (BD Biosciences Pharmingen), or 1 μg/ml anti-IL-10 (eBioscience) detection antibodies were added and incubated for 2 hours. After washing, streptavidin-horseradish:peroxidase (1:1000) was added for 1 hour. The plates were developed by adding 3-amino-9-ethylcarbazole (AEC) substrate kit (BD biosciences), and the resulting results were counted using a computer-assisted ELISPOT image analyzer (T Spot; Cellular Technology, Cleveland, Ohio).

Mouse real-time RT-PCR assay: Real-time (RT)-PCR was performed as previously reported (Azimzadeh, A. M. et at (2006) Transplantation 81, 255-264). Total RNA was isolated from cardiac grafts using the RNeasy mini kit from Qiagen (Valencia, Calif.). Briefly, tissue was disrupted in glass grinders in RLT buffer, homogenized using a Tissue Miser (Fisher Scientific, Cat # 1533855), digested with Proteinase K (Qiagen), loaded on Qiagen columns, and treated with DNase I (Qiagen). Purified RNA was quantified and assessed for purity and integrity by capillary electrophoresis using the Agilent Bioanalyzer. cDNA was generated from 3-6 μg of each RNA sample using SuperScript II RNase H-reverse transcriptase (Invitrogen, Carlsbad, Calif.) and a mix of oligodT and random primers in the ratio of 4:1 (Applied Biosystems, Foster City, Calif., and Invitrogen). 50 ng of the resultant cDNA was used in each PCR reaction. rpL-32 (ribosomal protein L32) was chosen as housekeeping gene control after testing the relative expression of PPIA (peptidylprolyl isomerase A), HPRT (Hypoxanthine guanine PhosphoRibosyl Transferase), and rpL-32 on normal and rejected mouse heart samples. Six experimental samples were excluded from the analysis due to poor RNA quality (Agilent RIN<2, in association with delayed amplification of the house-keeping gene). The primers and Taqman probe for rpL-32, PPIA, HPRT, IFN-γ, IL-4, IL-10, CTLA-4, TNF-α, iNOS (Nitric Oxide Synthase 2, inducible), and TGFβ-1 were kindly provided by Dr. Harry Dawson, and those for Foxp3, CD25, IDO (indoleamine-pyrrole 2,3 dioxygenase), IL-2, IL-12β, PD-1 (PdcI, Programmed Cell Death 1), Granzyme B, and FasL (Fas Ligand) were obtained from Applied Biosystems. The real-time PCR assay was performed on the ABI Prism 7900 (Applied Biosystems). The expression of each gene was normalized to the housekeeping rpL32 using the AACT calculation and mRNA levels were finally expressed as relative fold increase over native unmanipulated C57BL/6 heart tissue.

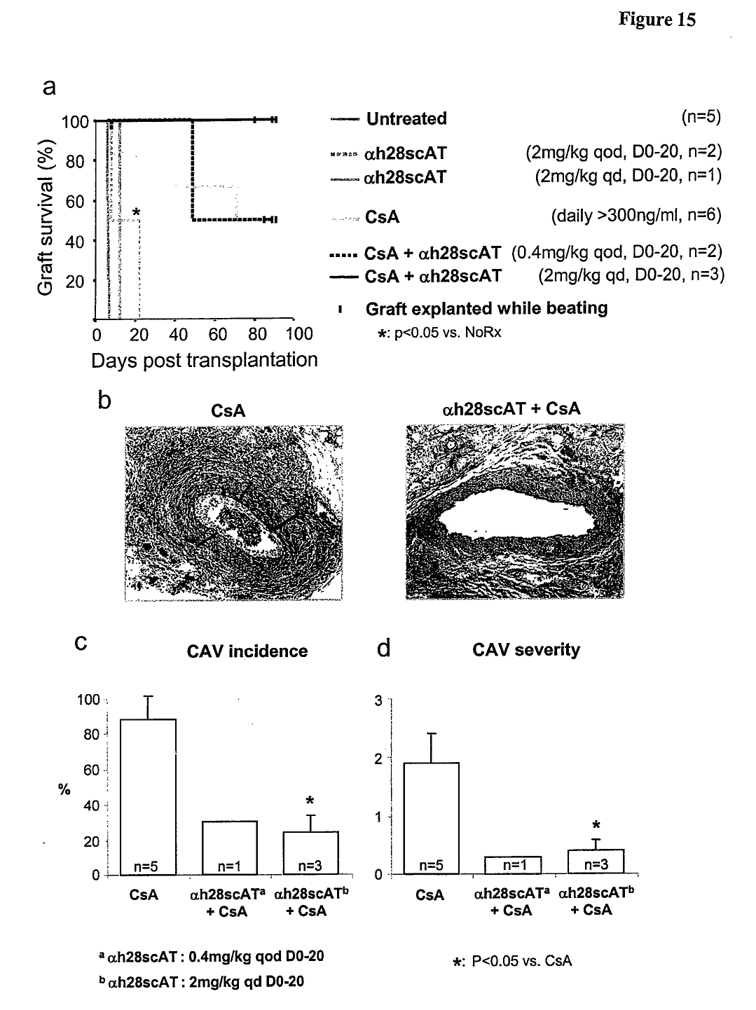

Cardiac transplantation in monkeys: All recipient animals underwent heterotopic intraabdominal cardiac allograft transplantation, as described previously (Pierson, R. N., III et al. (1999) Transplantation 68, 1800-1805; Azimzadeh, A. M. et al. (2006) Transplantation 81, 255-264). Reference groups were either untreated (n=5), or received cyclosporine A (CsA) (Neoral, Novartis, Hannover, N.J., n=6). CsA was given once daily (IM at 5-25 mg/kg) to achieve therapeutic target trough levels (>400 ng/ml). αh28scAT was given as indicated in FIG. 15a. Open cardiac biopsies were performed on postoperative days 7, 14, 28 and monthly thereafter until graft explant. Graft function was monitored daily by palpation and implanted telemetry (Data Sciences International, St. Paul, Minn.). Clinical acute graft rejection was detected as consistent high body temperature (>3 8.5° C.) coupled with either a decrease in graft heart rate (to <120 beats per min (bpm), or a drop of >40 bpm from a stable baseline) or an increase in graft diastolic pressure of >10 mmHg. Graft failure was defined as loss of contraction by telemetry and confirmed at explant, and was always preceded by signs of acute rejection. In two CsA-treated animals, a first episode of symptomatic acute rejection was treated with a three daily steroid boluses (Solu-Medrol®, Pharmacia, Kalamozoo, Mich.; 10 mg/kg). In one CsA-treated animal (M262), suspected rejection based on histological analysis of the biopsy tissue sample was also treated with a three day course of steroids. Cellular infiltrates were analyzed on H&E-stained paraffin sections, and graded for acute rejection by ISHLT criteria (Billingham, M. E. et al. (1990) J. Heart Transplant. 9, 587-593). CAV incidence in beating hearts explanted after day 70 was recorded as percent of arteries and arteriolar vessels involved (CAV score ≧1) at each time point. CAV severity was scored in these explanted hearts as follows: Grade 0, normal arterial morphology; Grade 1, activated endothelial cells with enlarged nuclei and/or adherent leukocytes, without luminal narrowing (<10%); Grade 2, distinct neointimal thickening, luminal narrowing <50%; Grade 3, extensive neointimal proliferation with greater than 50% luminal occlusion. Scoring was independently performed for each explanted heart by three evaluators (TZ, RNP, BN) blinded with respect to treatment group. The mean CAV score for each biopsy or explant was calculated using the equation: #grade 0−vessels×0+#grade 1−vessels×1+#grade 2−vessels×2+#grade 3−vessels×3)/total number of arterial vessels scored; and individual means averaged to calculate the group mean ±SD for each treatment group.

Statistical analysis: Graft survival time was expressed as the mean plus standard deviation and graphed with use of the Kaplan-Meier method. The log-rank test was used to compare survival time between different groups. Continuous variables were expressed as the mean plus standard deviation unless otherwise indicated and were compared using the Mann-Whitney non parametric test. Nominal variables (i.e. incidence of early rejection) were measured using a contingency table and the Chi-square test. P-values less than 0.05 were considered statistically significant. All statistical analyses were performed on a personal computer with the statistical package SPSS for Windows XP (Version 11.0, SPSS, Chicago, Ill., USA) or GraphPad InStat (version 5. 1, GraphPad Software, San Diego, Calif., USA).

Results

Anti-CD28 scFv Inhibits Lymphocyte Proliferation

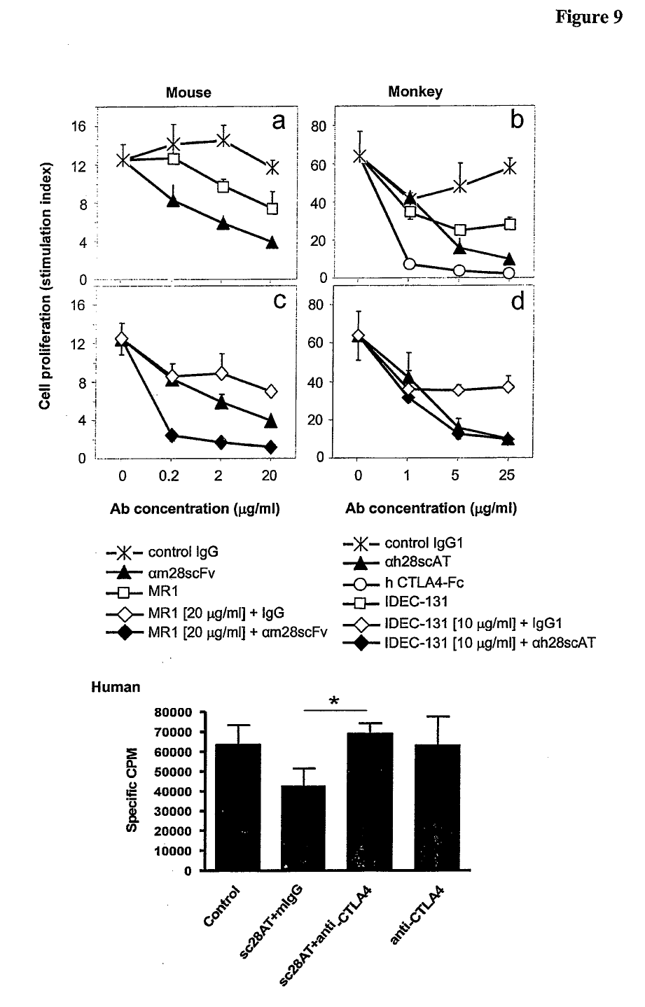

In most instances intact antibodies specific for the CD28 binding site for B7 deliver activating signals through CD28, clouding interpretation of heterogeneous effects associated with this approach (Nunes, J. et al. (1993) Int. Immunol. 5, 311-315). In contrast, monovalent recombinant single-chain (sc) antibody fragments containing the F-variable (Fv) region of a high-affinity anti-CD28 clone block CD28 binding to B7 without CD28 signaling (OHara et al. 2003 The FASEB Journal 17, C178 Abstract; Vanhove, B. et al. (2003) Blood 102, 564-570). Non-activating anti-mouse CD28 scFv antibody fragment (am28scFv) inhibited allogeneic lymphocyte proliferation in a mixed lymphocyte reaction (MLR) by 50-80% at 0.2-20 μg/ml, concentrations that are readily attainable in vivo (FIG. 9a). Whereas CD 154 blockade minimally affected mouse cell proliferation (FIG. 9a), an additive anti-proliferative effect was seen with additional αm28scFv relative to anti-CD28 or anti-CD154 alone (MR1, 20 μg/ml) (FIG. 9c). This additive effect was also observed using lower concentrations of MR1 (0.2 or 2 μg/ml, data not shown). Similarly, a non-activating anti-human CD28 scFv antibody fragment linked to alpha-I anti-trypsin (AT) to prolong its serum half-life (αh28scAT) (Vanhove, B. et al. (2003) Blood. 102, 564-570) inhibited alloreactive cynomologus lymphocyte proliferation in a dose-dependent fashion (FIG. 9b); in conditions where CD 154 blockade alone inhibited 20-40% of monkey cell proliferation (IDEC-131,10 μg/ml), an additive effect was observed with additional αh28scAT as compared to anti-CD154, but was not different from anti-CD28 alone (FIG. 9d). Inhibition of human allogeneic T cell proliferation by αh28scAT was antagonized by additionally blocking CTLA-4. Thus, part of the inhibition of alloproliferation by selective CD28 blockade is mediated by CTLA-4 in man requires intact CTLA-4/B7 interaction (FIG. 9e).

CD28 Blockade Prolongs Murine Cardiac Allograft Survival

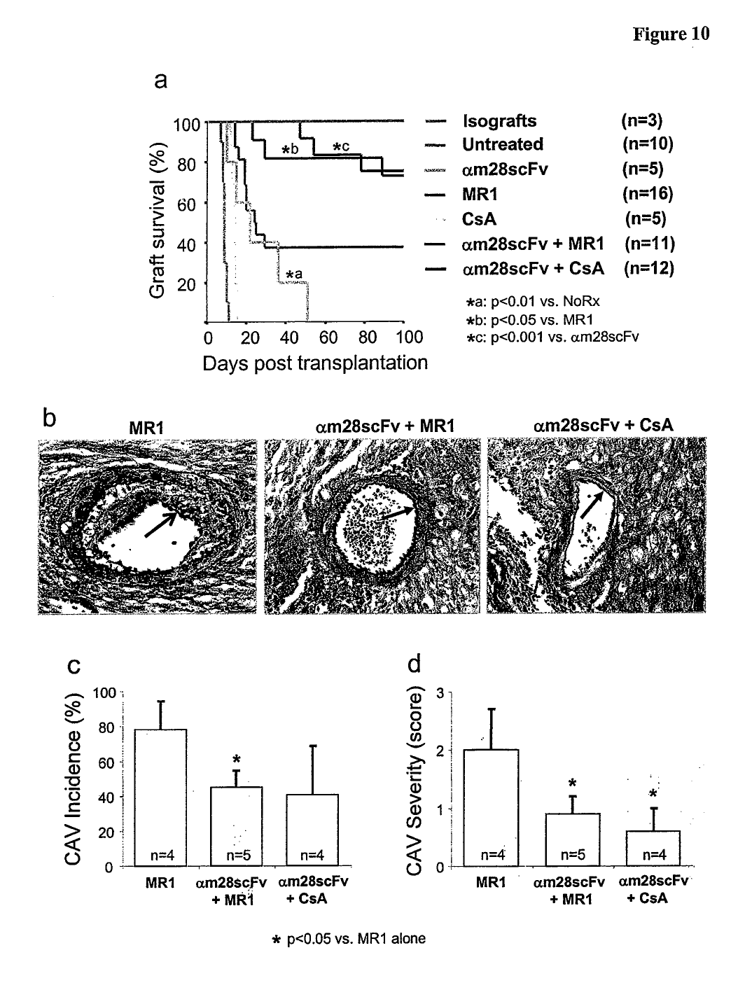

Induction monotherapy with twice daily intraperitoneal αm28scFv (200 μg/day) significantly prolonged survival of fully MHC-mismatched heterotopic murine cardiac allografts (FIG. 10). Whereas untreated BALB/c recipient mice rejected C57BL/6 cardiac allografts within 10 days (mean survival time (MST) 9.0 days, n=10), grafts in recipients treated with αm28scFv for 14 days rarely rejected during therapy, and had significantly prolonged graft survival (MST, 26.8 days; n=5; P<0.05). All allografts rejected within 51 days, demonstrating that this regimen does not induce tolerance across this full MHC disparity.

Graft acceptance is facilitated by transiently attenuating either calcineurin- or CD 154-dependent adaptive immune pathways at transplant in the context of aαm28 scFv induction regimen. Eight of eleven animals treated with αm28 scFv combined with a single injection of MR1 on the day of transplant had indefinite (>100 day) graft survival (P<0.05 vs. anti-CD28 or MR1 monotherapy). Similarly, αm28scFv combined with a three day peritransplant course of cyclosporin A (CsA) significantly prolonged graft survival, with 9 of 12 allografts surviving >100 days (p<0.05 compared to treatment with either agent alone) (FIG. 10a). Graft acceptance was mediated by CTLA-4, since addition of anti-CTLA-4 treatment during induction led to allograft with 10 days in all 6 μm28scFv-treated animals, 3 with CsA and 3 with MR1 (data not shown).

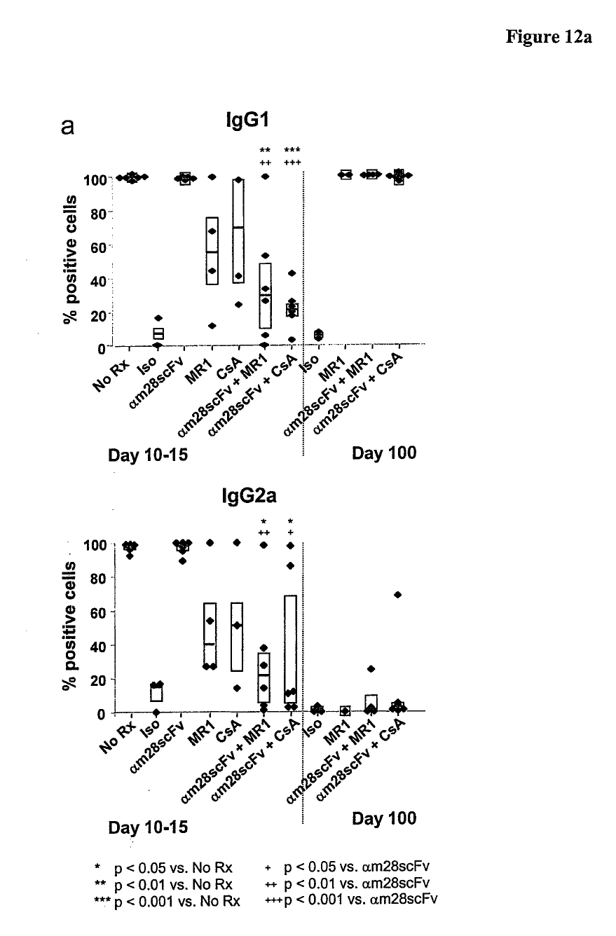

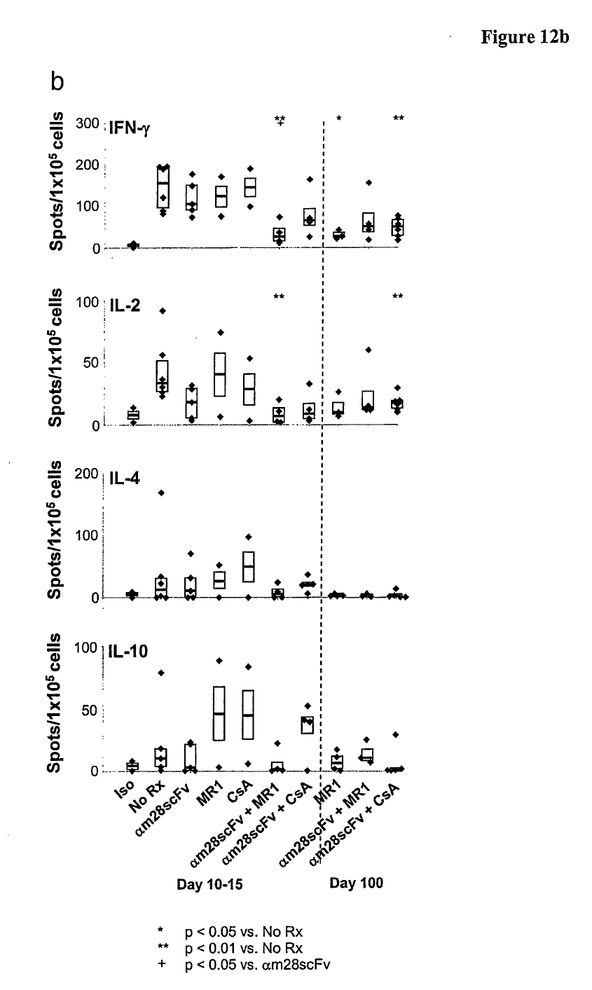

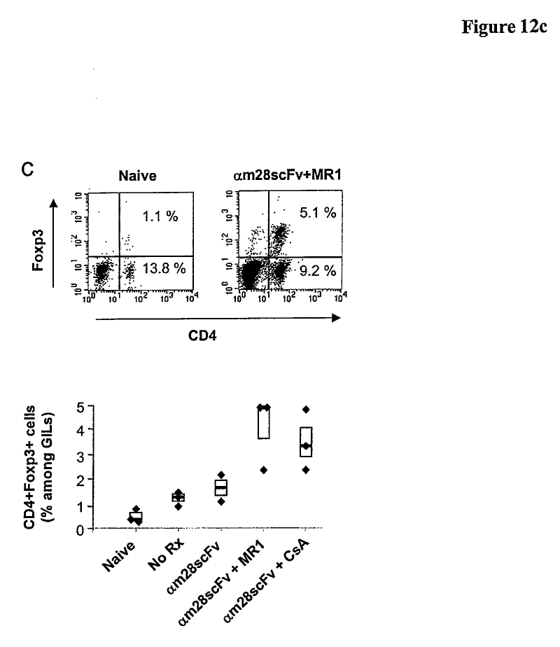

Cardiac allografts from untreated recipients exhibited diffuse mononuclear cell infiltration, myocyte necrosis and interstitial hemorrhage (ISHLT Grade 4) at the time graft function ceased. Grafts harvested by protocol 10-15 days after transplantation from CD28-treated mice exhibited focal lymphocyte aggregates and patchy myocyte injury (ISHLT grade 3A). Treatment with αm28scFv plus either CsA or MR1 was associated with sparse lymphocyte infiltration and preserved myocardial architecture (FIG. 11). At 10-15 days post transplantation, IgG1 and IgG2a alloantibody were consistently observed in all groups, although recipients treated with αm28scFv and either CsA or MR1 had significantly lower alloantibody levels (FIG. 12a).

At 100 days, all recipients with surviving grafts had high levels of IgG1, but IgG2a alloantibody levels were significantly lower than those observed in animals within two weeks after transplant (FIG. 12a). In surviving MR1-treated grafts (6 of 16) at this interval, 4 examined grafts exhibited mild or moderate cellular infiltrates (ISHLT Grade 1-3) but severe cardiac allograft vasculopathy (CAV) (FIG. 10b-d). In contrast, with αm28scFv plus MR1 (n=5) or CsA (n=4), graft infiltration was sparse (ISHLT Grade 0-1), cardiac morphology normal, and CAV mild. Quantified by lesion prevalence and severity, CAV was significantly attenuated with either combined regimen compared to MR1 alone (P<0.05) (FIG. 10d).

Skin Allograft Survival after Cardiac Allograft Acceptance

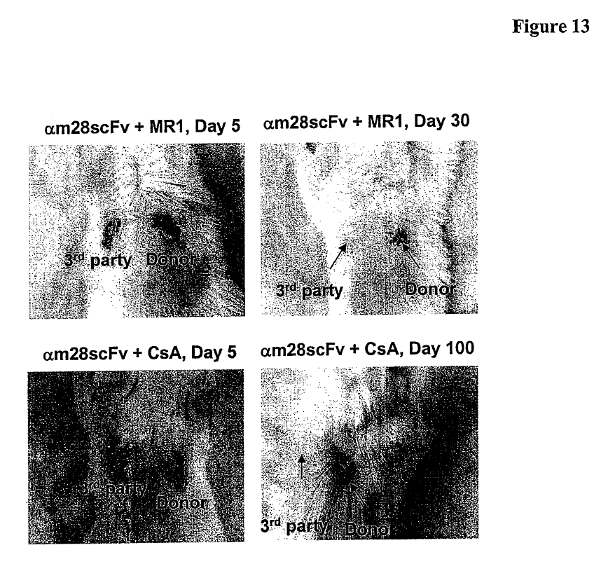

Donor-specific tolerance was assayed in mice with functioning allografts at day 100 after induction treatment with αm28scFv plus MR1 (n=3) or CsA (n=4), by challenging recipient mice with donor-type and third-party skin allograft without additional immunomodulatory treatment. While recipients in both groups promptly rejected third-party C3H skin grafts within 8 days, but donor-strain skin (C57BL/6) was rejected significantly more slowly (P<0.05 vs. third-party) (Table 2 and FIG. 13). Previously accepted cardiac grafts did not reject after skin transplantation in 1 of 3 αm28scFv plus MR1 and 2 of 4 αm28scFv plus CsA-treated animals (Table 2), all together suggesting that donor-specific immunoregulation was confined to the cardiac graft and could sometimes be overcome by sensitization with a skin graft.

TABLE 2
Skin graft survival in long-term heart graft-accepting recipients
and donor heart survival after skin transplant

| | C3H skin | B6 skin | P vs. | B6 heart |
|---|---|---|---|---|
| | 3^{rd} party, | donor type, | 3^{rd} | after skin |
| Treatment (n) | d | d | party | Tx, d |
| αm28scFv + | 5, 6, 6 | >35, 41, 76 | <0.05 | >35, >41, 55 |
| MR1 (3) | | | | |
| αm28scFv + | 5, 6, 7, 7 | 10, 12, 12, >100 | <0.01 | >40, 27, >51, 20 |
| CsA (4) | | | | |

Donor-Reactive Splenocyte Frequency

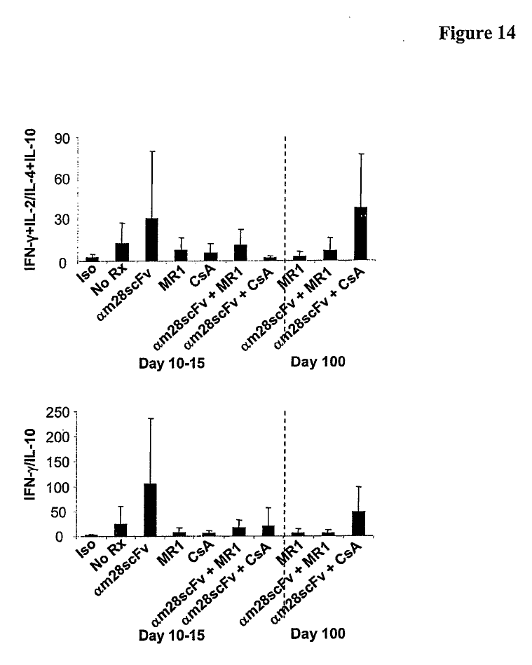

The frequency of donor-reactive splenocytes expressing Th1 and Th2 cytokines was assessed by ELISPOT in a small number of animals culled at day 10-15 after transplant, or after 100 days in animals with surviving grafts. Although treatment with αm28scFv combined with MR1 or CsA was associated with fewer IFN-γ— and IL-2-producing splenocytes relative to no treatment or αm28scFv monotherapy, donor-reactive cells were, however, consistently present at increased levels relative to naive animals and isograft recipients (<10 per 3×10⁵ cells) (FIG. 12b). Th1/Th2 ratios with αm28scFv plus CsA showed a trend toward early Th2 and late Th1 immune bias compared to αm28scFv plus MR1, where little change in bias was evident between these timepoints (FIG. 14). In aggregate, induction of tolerance by αm28scFv combined with transient CD154 costimulation blockade or calcineurin inhibition was associated with less expansion of donor-reactive splenocytes, and distinct late cytokine skewing.

Foxp3⁺ Cells Infiltrating Cardiac Allografts

Since donor-specific splenocytes producing IFN-γ, IL-2-, and IL-10 were readily detected at late follow-up in animals with accepted graftts, it was investigated whether induction of cardiac allograft acceptance was associated with expansion of donor-reactive Tregs. Expression of intracellular Foxp3, a transcription factor pivotal to the development and function of Tregs, was measured in splenocytes and graft infiltrating cells from transplant recipients treated with αm28scFv alone or in combined therapies at day 10-12 after transplant by flow cytometry. The proportion of Foxp3⁺ spleen CD4⁺ T cells in naive BALB/c mice (2.5±1.2%) was not affected by transplantation or treatment with αm28scFv-based therapies (data not shown). In contrast, the proportion of CD4⁺ Foxp3⁺ T cells in the cardiac allograft of mice treated with αm28scFv with CD154 (4.1±1.5%) or CsA (3.6±1.3%) was increased relative to a rejecting untreated cardiac allograft (1.2±0.3%) or untransplanted native heart (0.5±0.3%) (FIG. 12c). Foxp3 expression was associated with CD25 expression, and was minimal on CD4 negative cells (FIG. 12c). Thus, anti-CD28-based therapies that induce tolerance are associated with increased early graft infiltration by CD4⁺ CD25⁺Foxp3⁺T cells.

Gene Expression Profiles During Induction and Maintenance of Tolerance

Expression of genes associated with T-cell or dendritic cell activation and regulation were quantified by real-time RT-PCR in surviving cardiac grafts at 100 days post-transplantation. Relative to normal mouse hearts (naive control) or isografts, expression of Th1 (IFN-γ, IL-2) and Th2 cytokine genes (IL-4, IL-10), TGF-β, TNF-α, iNOS and Granzyme B were expressed to a similar degree in allografts with stable late tolerance (αm28scFv with MR1 or CsA) or chronic rejection (MR1). Foxp3, CTLA-4, IL2R^{A}, FasL, and PD-1, remained increased at day 100 and tended to be higher in grafts from tolerant animals relative to MR1-treated grafts with chronic rejection. In contrast, IDO was particularly enriched in grafts from recipients treated with αm28scFv+CsA (p=0.03) and tended to be increased with αm28scFv+CD154, compared to those treated with MR1 alone.

αh28scAT and Primate Cardiac Allograft Immunity

In cynomolgus macaques treated with αh28scAT monotherapy at 2 mg/kg every other day (qod, n=2) or daily (qd, n=1), cardiac allografts survived for 8, 14, and 22 days (MST 14±7 days), significantly longer than in untreated monkeys (MST 6.4±0.4 days; n=5; p=0 01, (Schroeder, C. et al. Journal of Immunology 2 A.D: Unpublished Work) (FIG. 15a). In one animal treated with αh28scAT, moderate acute cellular rejection (ISHLT Grade 2-3A) on day 7 receded in the subsequent biopsy at day 14. All grafts failed due to acute cellular rejection despite ongoing anti-CD28 monotherapy.

CsA (Neoral) was dosed at 10-25 mg/kg IM daily to achieve therapeutic trough levels >400 ng/ml (Schroeder, C. et al. Journal of Immunology. 2 A.D.: Unpublished Work; Schuurman, H. J. et al. (2001) Transpl. Int. 14, 320-328). Three of six animals exhibited symptomatic acute allograft rejection (graft bradycardia and/or diminished contractility, recipient fever) on days 7, 23 and 71. One graft was explanted (steroid rescue was not attempted in this case), the other two responded to treatment with steroids, and underwent explantation of functioning grafts on days 72 and 92, respectively. Three other grafts without clinical rejection were electively explanted around day 90 (Table 3).

TABLE 3
Individual graft survival time and histological analysis of
monkey cardiac allografts treated with various regimens.

| | | Primary | Secondary | | | | | | | | |
|---|---|---|---|---|---|---|---|---|---|---|---|
| | | survival | survival | Biopsy score (POD) | | | | | | | Explant |
| Group | Monkey | (days)^{a} | (days) ^{b} | 7 | 14 | 28 | 35 | 56 | 63 | 84 | score |
| NoRx | M360 | 6 | | | | | | | | | 3A |
| | M364 | 6 | | | | | | | | | 3A |
| | M20 | 6.5 | | | | | | | | | 4 |
| | M278 | 6.5 | | | | | | | | | 3B-4 |
| | M342 | 7 | | | | | | | | | 3B |
| ah28scAT | M9395 | 8 | | | | | | | | | 4 |
| 2 mg/kg qod˜day 20 | M9394 | 22 | | 2-3A | 1B | | | | | | 4 |
| ah28scAT | M9398 | 12 | | 1B-2 | | | | | | | 4 |
| 2 mg/kg qd˜day 20 | | | | | | | | | | | |
| CsA | M162 | 7 | | | | | | | | | 4 |
| daily >300 ng/ml | M9421 | 23 | 72 | 2 | 1A | | 3B | 3B | | | 2 |
| | M115 | 71 | >92 | 0 | | | | 1A-2 | | 3B | 4 |
| | M262 | >85 | | 1A | | | 0 | 1A | | | 2-3A |
| | MA095 | >89 | | 1A | 1A-2 | | 0 | | 3B-4 | | 3A |
| | MA049 | >91 | | 0-1A | 1A | | 3A | | 1B-2 | | >91 |
| CsA + ah2βscAT | M9393 | 49 | | 1A | 1B | | 1B | | | | 4 |
| 0.4 mg/kg qod˜day 20 | M9400 | >89 | | 1A | 1A | | 3B | 1A | | | 0-1A |
| CsA + ah28scAT | M9429 | >80 | | 1A-1B | 1B | 3A | | 0-1A | | | 1A |
| 2mg/kg qd˜day 20 | M9411 | >89 | | 1A | 2 | 1B-2 | | 1A | | | 0 |
| | MA086 | >91 | | | 0 | 0 | | | 0-1A | | 2-3A |

As shown in Table 3, graft survival time indicates the time at which the graft was explanted because of rejection, except for >which represents grafts explanted while beating for technical or animal health reasons. When a first episode of clinical rejection was treated with steroids, primary survival time represents the time of rejection treatment (^{a}) and secondary survival time indicates the time at which the graft was explanted (^{b}). Rejection scores were determined by analysis of H&E sections from biopsy and explanted cardiac allograft tissue according to the ISHLT criteria (Azimzadeh et al. (2006) Transplantation 81:255-264) as previously described (Billingham et al. (1990) J. Heart Transplant. 9:587-593).

When CsA was combined with αh28scAT, one of two animals treated with a low dose αch28scAT (0.4 mg/kg daily) and therapeutic CsA exhibited symptomatic acute rejection at day 47 that was not treated and progressed to graft failure. In contrast none of four animals treated with αh28scAT at 2 mg/kg daily or the other recipient given subtherapeutic αh28scAT developed symptomatic rejection. One animal (M9429) was euthanized at day 80 due to a lymphoma and two grafts without clinical rejection were electively explanted.

ISHLT rejection scores were consistently lower on protocol biopsies and at graft explant from monkeys treated with αh28scAT+CsA versus CsA alone (Table 3) When moderate acute cellular rejection (ISHLT Grade ≧2) was observed in αh28scAT-treated grafts, the infiltrate receded in each of three instances (M9400, d35; M9429, d28; M9411, d14), even when αh28scAT had been discontinued 7 or 14 days previously. These observations demonstrate active, clinically important regulation of anti-donor immunity across a full MHC mismatch in primates. Importantly, whereas all grafts treated with CsA monotherapy exhibited severe cardiac allograft vasculopathy (CAV) at explant, the CAV score associated with CD28 inhibition dosed at 0.4 mg/kg (CAV score=0.3, n=2) and 2 mg/kg daily (0.4±0.2, n=3) was significantly reduced relative to therapeutic CsA alone (1.9±0.5, n=5; p=0.04) (FIG. 15c-d).

Discussion

The results disclosed herein show that selectively blocking CD28 using a monovalent non-activating scFv reagent significantly modulated the immune response to MHC antigens in both mice and monkeys. Induction monotherapy with αm28scFv or αh28scAT monotherapy attenuated the pace of acute cardiac allograft rejection in the context of evanescent graft infiltrates that reflect regulation within the transplanted organ of an active response to donor antigens. CD28-driven events occurring within the first weeks after transplant were pivotal to the severity of subsequent cardiac allograft vasculopathy both in mice and in monkeys. During CD28 blockade in the mouse, the initial donor-host interaction was associated with a vigorous expansion of donor-reactive T-cells in the spleen; this population persisted or regenerated for months therafter. Pathogenic alloimmunity was efficiently attenuated by an additional short course of peritransplant CD 154 inhibition or CsA. Protection from allograft injury was mediated by CTLA4, and was associated with modulation of an Th1 (but not Th2) antibody response and mild CAV long after discontinuation of treatment. The host retained detectable if incompletely effective systemic donor-specific regulatory function, since animals with surviving heart allografts three months after CD28 induction demonstrated prolonged survival of subsequent donor skin grafts, but prompt rejection of third-party skin. Retention of some heart grafts despite delayed rejection of donor skin shows that transient selective blockade of CD28 promoted establishment of durable organ-specific tolerance.

The mechanism of initial graft protection and subsequent acceptance was not primarily via a Th2 bias, as shown in several other models of peripheral tolerance (Strom, T. B. et al. (1996) Curr. Opin. Immunol. 8, 688-693; Chen et al. (1996) Transplantation 61, 1076-1083; Kishimoto, K. et al. (2002) J. Clin. Invest 109, 1471-1479; Dallman, M. J. et al. (1993) Immunol. Rev. 133:5-18, 5-18). Further, Thbias did not obviously account for protection from CAV, since similar splenic ELISPOT cytokine profiles, intra-graft gene expression phenotypes and alloantibody titers were found in MR1-treated animals with severe graft CAV, and in animals treated with either anti-mCD28-based approach, which exhibited relatively mild CAV.

Foxp3 is a transcription factor important in the development and function of CD4+CD25+ T regs. As described herein, an increase in Foxp3 gene expression was observed and Foxp3+ cells were found within the graft during tolerance induction. However, neither Foxp3 expression nor a panel of other regulatory T-cell genes (CTLA4, TGF-β, IL-10, IL-2R^{A}) individually distinguished tolerant from chronically rejecting grafts at 100 days after transplant. Rather, addition of αm28scFv to MR1 or CsA was associated with a trend towards enhanced expression of CTLA4, FasL, PD-1, and IDO in the graft relative to MR1 alone, suggesting that CD28 blockade promotes coordinated evolution of both T-cell and DC protective mechanisms within the graft.

The disclosure described herein is consistent with the general hypothesis that CTLA-4 is pivotal to regulatory T-cell expansion in response to allogeneic stimulation (FIG. 9e), and to induction of peripheral tolerance, as previously suggested (Zheng, X. X. et al. (1999) J. Immunol. 162, 4983-4990; Tsai, M. K. et al. (2004) Transplantation 77:48-54; Markees, T. G. et al. (1998) J. Clin. Invest. 101, 2446-2455; Chandraker, A. et al. (2005) Transplantation. 79, 897-903). Increased numbers of CD4+ Foxp3 T-cells in accepted grafts are consistent with studies from other murine peripheral transplant tolerance models (Graca, L. et al. (2002) J. Exp. Med. 195:1641-1646; Chen and Bromberg (2006) Am. J. Transplant. 6:1518-1523), but do not exclude a role for non-T regulatory cells, as observed after treatment with a modulating anti-CD28 antibody. Membrane-bound CTLA4, induced or upregulated on T-cells after T cell receptor ligation and partial costimulation through other available pathways (e.g. CD27/CD70, HVEM/LIGHT) (Ansari, M. J. & Sayegh, M. H. (2006) J. Clin. Invest. 116, 2080-2083) ligates B7 receptors on DC to induce IFN-γ-dependent up-regulation of indoleamine 2,3-dioxygenase (IDO), a tryptophan-catabolizing enzyme associated with immunosuppressive activity (Mellor, A. L. & Munn, D. H. (2004) Nat. Rev. Immunol. 4, 762-774). CTLA-4/B7 molecular interactions may mediate improved allograft survival in mouse recipients treated with ocm28 scFv by increasing IDO transcription and thus regulatory function in graft DCs (Fallarino, F. et al. (2003) Nat. Immunol 4, 1206-1212; Finger, E. B. & Bluestone, J. A. (2002) Nat. Immunol. 3, 1056-1057). Like CTLA-4, PD-1 negatively regulates T-cell activation and its expression tends to be increased in accepted grafts relative to those with chronic rejection. Recent studies demonstrated that CTLA-4 and PD- I cooperate to maintain CD8 peripheral tolerance (Probst et al. (2005) Nat. Immunol. 6, 280-286) and inhibit T-cell activation through distinct and potentially synergistic biochemical mechanisms. Current studies to block CTLA-4 or PD-I at later intervals after anti-mCD28-based induction treatment will test whether these pathways are necessary to maintenance of donor-specific peripheral immunoregulation.

Therefore, as described herein, CTLA4 plays a pivotal role to induce the relative expansion of donor-specific CD4⁺CD25⁺Foxp3 Treg cells when CD28 is blocked. Alloreactive T regs then actively modulate pathogenic cytotoxicity and T-helper-facilitated antibody elaboration, direct anti-inflammatory maturation events in donor and recipient DCs, and thus promote prolonged allograft survival by induction of regulatory DCs in the graft. The efficacy of anti-CD28 with conventional immunosuppression to inhibit chronic rejection in primates as well as mice is promising for potential clinical application. While further work will be required to dissect the mechanisms responsible and define clinically useful regimens, the presented studies confirm initial hypothesis that non-activating CD28 blockade can modulate alloimmunity by active CTLA4-dependent process. Whether selective monovalent CD28-directed therapy has significant practical advantages relative to B7 blockade (Vincenti, F. et al. (2005) N. Engl. J. Med. 353, 770-781; Adams, A. B. et al. (2002) Diabetes 51, 265-270; Pearson, T. C. et al. (2002) Transplantation 74, 933-940, as this model predicts, remains to be formally tested.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of prolonging graft survival in a subject in need thereof comprising administering to the subject a non-activating anti-CD28 antibody that blocks CD28 binding to B7 without CD28 signaling such that graft survival in the subject is prolonged.

2. The method of claim 1, wherein the subject in need thereof is a transplant recipient.

3. The method of claim 1, wherein the graft is an allograft.

4. The method of claim 1, wherein the allograft is a cardiac, liver, lung, kidney or pancreatic allograft.

5. The method of claim 1, wherein the non-activating anti-CD28 antibody is an immunologically active fragment.

6. The method of claim 1, wherein the non-activating anti-CD28 antibody is a Fab, F(v), Fab′, or F(ab′)₂.

7. The method of claim 1, wherein the non-activating anti-CD28 antibody is a single chain antibody.

8. The method of claim 1, wherein the non-activating anti-CD28 antibody is a single chain F(v) (scFv).

9. The method of claim 8, wherein the anti-CD28 scFv is linked to an agent to prolong its serum half-life.

10. The method of claim 9, wherein the agent used to prolong serum half-life is polyetheylene glycol.

11. The method of claim 9, wherein the agent used to prolong serum half-life is alpha-1 anti-trypsin.

12. The method of claim 1, wherein the non-activating anti-CD28 antibody is humanized.

13. The method of claim 1, wherein the non-activating anti-CD28 antibody is fully human.

14. The method of claim 1, further comprising administering an immunosuppressive drug.

15. The method of claim 14, wherein the immunosuppressive drug is selected from the group consisting of: methotrexate, rapamycin, cyclosporin, FK506, an anti-CD154 antibody, a steroid, a CD40 pathway inhibitor, a transplant salvage pathway inhibitor, a IL-2 receptor antagonist, and analogs thereof.

16. The method of claim 14, wherein the immunosuppressive drug is cyclosporine A.

17. The method of claim 14, wherein the immunosuppressive drug is an anti-CD154 antibody.

18. The method of claim 17, wherein the anti-CD154 antibody is MR1.

19. A method of treating type I diabetes in a subject in need thereof comprising administering to the subject a non-activating anti-CD28 antibody that blocks CD28 binding to B7 without CD28 signaling, thereby treating type I diabetes in the subject.

20. The method of claim 19, wherein the non-activating anti-CD28 antibody is a Fab, F(v), Fab′, or F(ab′)₂.

21. The method of claim 19, wherein the non-activating anti-CD28 antibody is a single chain antibody.

22. The method of claim 19, wherein the non-activating anti-CD28 antibody is a scFv.

23. The method of claim 22, wherein the anti-CD28 scFv is linked to an agent to prolong its serum half-life.

24. The method of claim 19, further comprising administering an immunosuppressive drug.

25. The method of claim 19, wherein the immunosuppressive drug is selected from the group consisting of: methotrexate, rapamycin, cyclosporin, FK506, an anti-CD154 antibody, a steroid, a CD40 pathway inhibitor, a transplant salvage pathway inhibitor, a IL-2 receptor antagonist, and analogs thereof.

26. A method of treating type I diabetes in a subject comprising administering an effective amount of spleen cells from a donor subject treated with an antigen binding portion of anti-CD28 antibody that blocks signaling via CD28 to the subject, thereby treating type I diabetes in the subject.

27. The method of claim 26, wherein the subject is a mammal.

28. The method of claim 26, wherein the subject is a human.

29. The method of claim 26, wherein the antigen binding portion is a scFV or a Fab fragment.

30. The method of claim 26, wherein the antigen binding portion is a scFV.

31. The method of claim 26, wherein the scFV is PV1.

32. The method of claim 26, wherein the antigen binding portion is humanized.

33. The method of claim 26, wherein the antigen binding portion is fully human.

34. The method of claim 26, wherein the spleen cells are administered to the subject by injection.

35. The method of claim 26, further comprising administering an immunosuppressive drug.

36. The method of claim 35, wherein the immunosuppressive drug is selected from the group consisting of: methotrexate, rapamycin, cyclosporin, FK506, an anti-CD154 antibody, a steroid, a CD40 pathway inhibitor, a transplant salvage pathway inhibitor, a IL-2 receptor antagonist, and analogs thereof.


SEQUENCE LISTING (2 sequences)

SEQ ID NO: 1 - DNA, 3803 bp, Homo sapiens
taaagtcatcaaaacaacgttatatcctgtgtgaaatgctgcagtcaggatgccttgtggttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaggaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatgcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaatggagataatggttacaaatgtctcttcctatagtataatctccataagggcatggcccaagtctgtctttgactctgcctatccctgacgtttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccatatcgaaaatactgtactttggttgatttttaagtgggcttccattccatggatttaatcagtcccaagaagatcaaactcagcagtacttgggtgctgaagaactgttggatttaccctggcacgtgtgccacttgcccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactact

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS